(19) United States
(12) Patent Application Publication
(10) Pub. No.: US 2004/0138296 A1
(43) Pub. Date: Jul. 15, 2004

(54) Amyloid immunization and Cox-2 inhibitors for the treatment of alzheimer's disease

(75) Inventors: David W. Robertson, Glenview, IL (US); Grant A. Krafft, Glenview, IL (US)

Correspondence Address: PHARMACIA CORPORATION, GLOBAL PATENT DEPARTMENT, POST OFFICE BOX 1027, ST. LOUIS, MO, 63006, US

(73) Assignee: Pharmacia Corporation

(21) Appl. No.: 10/627,357

(22) Filed: Jul. 25, 2003

Related U.S. Application Data
(60) Provisional application No. 60/402,760, filed on Aug. 12, 2002.
(60) Provisional application No. 60/402,778, filed on Aug. 12, 2002.
(60) Provisional application No. 60/402,674, filed on Aug. 12, 2002.
(60) Provisional application No. 60/402,655, filed on Aug. 12, 2002.
(60) Provisional application No. 60/402,773, filed on Aug. 12, 2002.
(60) Provisional application No. 60/402,675, filed on Aug. 12, 2002.
(60) Provisional application No. 60/402,676, filed on Aug. 12, 2002.

(51) Int. Cl. A61K 31/4709; A61K 31/19

(52) U.S. Cl. 514/461; 514/467; 514/314; 514/568

(57) ABSTRACT

The present invention provides compositions and methods for the treatment or prevention of Alzheimer's disease. More particularly, the invention provides a combination therapy for the treatment or prevention of Alzheimer's disease, wherein the therapy comprises administering to a subject an amyloid beta vaccine in combination with a cyclooxygenase-2 selective inhibitor.

### CROSS-REFERENCE TO RELATED PATENT APPLICATION

This patent claims priority to U.S. Provisional Application Serial No. 60/402,760, filed Aug. 12, 2002, U.S. Provisional Application Serial No. 60/402,778, filed Aug. 12, 2002, U.S. Provisional Application Serial No. 60/402,674, filed Aug. 12, 2002, U.S. Provisional Application Serial No. 60/402,676, filed Aug. 12, 2002, U.S. Provisional Application Serial No. 60/402,655, filed Aug. 12, 2002, U.S. Provisional Application Serial No. 60/402,773, filed Aug. 12, 2002, and U.S. Provisional Application Serial No. 60/402,675, filed Aug. 12, 2002. The entire text of this provisional application is incorporated by reference into the present application.

### FIELD OF THE INVENTION

The present invention provides compositions and methods for the treatment of Alzheimer's disease. More particularly, the invention is directed toward a combination therapy for the treatment or prevention of Alzheimer's disease comprising administering to a subject a cyclooxygenase-2 inhibitor in combination with amyloid vaccination.

### BACKGROUND OF THE INVENTION

Alzheimer's disease (AD) causes progressive dementia with consequent formation of amyloid plaques, neurofibrillary tangles, gliosis and neuronal loss. As one of the leading causes of death in industrialized countries, AD affects 5-11% of the population over the age of 65 and 30% of those over the age of 85. The estimated cost of caring for the approximate 2.5-4.0 million AD patients in the U.S. exceeded $60 billion in 1991 alone. It is further estimated that AD related costs would dramatically increase worldwide as the geriatric population grows.

Alzheimer's disease occurs in both genetic and sporadic forms, however clinical course and pathological features of both forms are quite similar. Three genes have been discovered which, when mutated, cause an autosomal dominant form of Alzheimer's disease. These encode the amyloid protein precursor (APP) and two proteins, presenilin-1 (PS1) and presenilin-2 (PS2), which are structurally and functionally related. Different forms of APP have been isolated and range in size from 695-770 amino acids, but all of them localize to the cell surface and have a single C-terminal transmembrane domain. Examples of specific isotypes of APP currently known to exist in humans include the 695-amino acid polypeptide described by Kang et. al. (1987), Nature 325: 733-736 which is designated as the “normal” APP; the 751 amino acid polypeptide described by Ponte et al. (1988), Nature 331: 525-527 (1988) and Tanzi et al. (1988), Nature 331: 528-530; and the 770 amino acid polypeptide described by Kitaguchi et. al. (1988), Nature 331: 530-532.

Mutations in any of the three proteins (APP, PS1 or PS2) have been observed to enhance proteolytic processing of APP via an intracellular pathway that produces amyloid beta peptide (Aβ peptide, or sometimes here as Abeta), a peptide that is the primary component of amyloid plaques in AD. Naturally-occurring β-amyloid peptide shows some heterogeneity since it can be 39-43 amino acid residues in length but generally it begins at an aspartic acid position 672 of APP-770.

The Aβ peptide is derived from a region adjacent to and containing a portion of the transmembrane domain of APP. Normally, processing of APP at the α-secretase site cleaves the midregion of the Aβ sequence adjacent to the membrane and releases the soluble, extracellular domain of APP from the cell surface. This α-secretase APP processing creates soluble APP-α, which is normal and not thought to contribute to AD. Pathological processing of APP at the β- and y-secretase sites, which are located N-terminal and C-terminal to the α-secretase site, respectively, produces a very different result than processing at the a site. Sequential processing at the β- and γ-secretase sites releases the Aβ peptide, and can occur in both the endoplasmic reticulum (in neurons) and in the endosomal/lysosomal pathway after reinternalization of cell surface APP (in all cells).

The amyloid plaque is the focus of complex cellular reactions involving the activation of both microglia and astrocytes adjacent to the amyloid plaque. Microglia are the most abundant and prominent cellular component of the plaque, where they generally exhibit a “reactive” or “activated” phenotype. Microglia are the principal immune cells in the brain, and are morphologically and functionally indistinguishable from macrophages. As seen with macrophages, the activated phenotype of microglia is associated with elevated expression of a number of cell surface molecules, including MHC class II antigens, CD45, complement receptors CR3 and CR4, immunoglobulin receptors FcgRI and FcgRII, and ICAM-1. Furthermore, activated microglia, like activated macrophages, secrete a diverse range of acute phase proteins including α-antichymotrypsin, α-antitrypsin, serum amyloid P, C-reactive protein, and complement components, among others (McGeer and Rogers, Neurology 42:447 [1992]). Importantly, activation of microglia results in the synthesis and secretion of proinflammatory cytokines IL-1β, IL-6, and TNF-α and macrophage chemotactic protein-1.

In addition to the above-mentioned features, a substantial decrease in cholinergic functioning has been well-documented in AD patients. Both the content of acetylcholine and the activity of choline acetyltransferase are reduced due to degeneration in the basal forebrain. Consistent with the “cholinergic hypothesis of AD”, which states that there is a direct relationship between the loss of cholinergic function in the brain and the degree of cognitive impairment (Bartus et al., Science, 217:408-414, 1982), anticholinergic drugs are known to impair memory and cognitive functioning in a similar fashion as AD (Sunderland et al., Arch Gen Psych, 44:418-425, 1987). This correlation has provided cues for potential AD therapies.

Accordingly, one of the current treatments for AD involves the use of cholinesterase inhibitors, also known as anticholinesterases. These agents inhibit the hydrolytic degradation of acetylcholine by the enzyme acetylcholinesterase (AchE) in the synaptic cleft, thus potentiating cholinergic transmission (Norberg A. and Svensson A. L., Drug Saf, 19:465-480, 1998). Tacrine (Cognex), a nonselective reversible cholinesterase inhibitor was the first drug in this class approved by the FDA for use in AD in 1993. However, this drug has a short half-life, requiring multiple daily doses and also exhibits hepatotoxic effects in a number of patients. Donepezil (Aricept) was approved in 1996 and has a longer half-life, allowing for once/day dosing and shows almost no hepatotoxicity and relatively low incidence of gastrointestinal side effects. It is now widely utilized to treat patients with mild to moderately severe AD patients since controlled trials have shown that the drug can delay AD-associated deterioration.

Rivastigmine is a relatively selective, pseudo-irreversible cholinesterase inhibitor with a 10-hour duration of action (Forerte et al., European J Neurol., 6:423-429, 1999); however, it does exhibit some gastrointestinal side effects and weight loss. Galantamine is a reversible competitive inhibitor as well as a modulator of nicotinic cholinergic receptors, and is currently approved for use for AD in Austria (Schenk et al., Abstracts from the 7^{th} International Conference on Alzheimer's Disease and Related Disorders, Neurobiol of Aging, 21(1S)S134, 2000). Other cholinesterase inhibitors are either in clinical trials or have been withdrawn from consideration due to adverse effects. For example, metrifonate was withdrawn from consideration during Phase III trials, where it was found to cause leg cramps and muscle cramps (Morris et al., Neurology, 50:1222-1230, 1998).

Some of the other treatments are based on the fact that monoamine oxidase B (MAO-B) activity is increased in AD and may cause an increase in oxidative deamination of monoamines. As a result of such deamination, hydrogen peroxide and other free radicals may be formed, resulting in toxic effects on neuronal membranes and loss of neurons. Selegiline, a selective MAO-B inhibitor, and alpha-tocopherol (vitamin E) are both antioxidants and appear to have therapeutic effect on AD treatment. In a study enrolling over 300 AD patients, selegeline and vitamin E were both found to slow the progression of the disease (Sano et al., NEJM, 336:1216-1222, 1997).

As mentioned previously, the lesions of AD are characterized by the presence of numerous inflammatory proteins. Accordingly, a number of studies have started to evaluate the efficacy of anti-inflammatory drugs in treatment of AD. For example, a controlled 6-month investigation by Rogers, J. et al., in Neurology (August 1993, 43:1609) involved the administration of 100-150 mg indomethacin (a non-steroidal anti-inflammatory drug, NSAID) or placebo to mild or moderately impaired Alzheimer's disease patients. The study concluded, based on a battery of cognitive tests, that the indomethacin treatment appeared to protect the patients receiving indomethacin from the degree of cognitive decline exhibited by the patients receiving placebo. Furthermore, S-2474, an NSAID that inhibits cyclooxygenase-2 significantly prevented neurons from Abeta (25-35) and Abeta (1-40) induced cell death (Yagami et al., British Journal of Pharmacology, 134(3):673-681, October 2001). Kadoyama et al. used mouse neuroblastoma and rat glioma hybrid NG108-15 cells to examine the role of COX-2 in APP production and secretion. For the experiment, they either mock-transfected the cells or stably transfected them with human Cox-2. Cells expressing Cox-2 exhibited 3-to 4-fold increases in both COX activity and prostaglandin E2 production. Notably, the mRNA level of amyloid precursor protein (APP) was elevated by approximately 2-fold in the Cox-2 expressing cells. In the same study, a selective Cox-2 inhibitor (JTE-522) and a nonselective Cox inhibitor (indomethacin) suppressed production of amyloid β-peptide and a secreted form of APP by inhibition of APP mRNA level (Kadoyama et al., Biochem. Biophys. Res. Commun., 281(2):483-490, 2001).

An alternative treatment that is currently in development involves vaccination with a synthetic form of the naturally occurring β-amyloid protein. In animals, immunization of young mice with Abeta prevented the appearance of amyloid plaques and other neuropathologic changes characteristic of AD (Reisberg et al., Neuobiol of Aging, 21(1S)S275, 2000). In addition, a single dose of an investigational Abeta vaccine (AN-1792) was well tolerated in 24 patients with early onset AD during a six-week period following injection (Schenk et al., Nature 400 (6740), pp.173-177, 1999). It is unclear how vaccination confers protection against AD but it is believed that the mechanism may involve 1) production of anti-β amyloid antibodies that can neutralize or deplete Abeta and/or 2) activation of microglia that can phagocytose deposited Abeta (Morgan et al., Nature, Vol. 408, no. 21, p. 982-985, December 2000). The hypothesis about activation of microglia is not as widely accepted as the hypothesis of antibody production since relatively modest Abeta clearance has been detected following vaccination.

A study by Casamenti et al. examined the effect of Cox-2 inhibitors on brain inflammation caused by an injection of pre-aggregated Abeta (1-42) into nucleus basalis (NB) of adult rats (Casamneti et al., J. Neurochem., 77, Suppl. 1, 10, 2001). In the experiment, rofecoxib attenuated microglial and astrocytic activation. As reported, however, Abeta vaccine was administered directly into the central nervous system (CNS). Another study by the same group (Scali et al., Society for Neuroscience Abstracts, Vol. 26, No. 1-2, 2000, ISSN:0190-5295) investigated the role of non-selective (ibuprofen) and selective Cox-2 inhibitors (rofecoxib and nimesulide) on glia reaction, inducible nitric oxyde synthase (iNOS) production, mitogen activated protein kinase (MAPK) expression and prostaglandin E2 (PGE2) levels during brain inflammation. The inflammatory reaction was induced either by injecting excitotoxin quisqualic acid (QUIS) or beta-amyloid peptide (1-42) intracerebrally. Once again, the Abeta injection was administered directly into the CNS. Seven days following the injection, both nimesulide and ibuprofen treatment (each administered once a day) attenuated microglia reaction and reduced the number of iNOS-positive cells but had no effect on astrocytic reaction. This is in contradiction with the previously-mentioned study where a selective Cox-2 inhibitor was able to attenuate astrocytic reaction. In addition, it is unclear whether the brain inflammation caused by the injection of QUIS or Abeta exhibits the same characteristics as observed in AD. Thus, it is difficult to determine from these studies the exact effects of Cox-2 inhibitors or amyloid beta peptide injection on Alzheimer's disease.

It is clear from the presented data that there is a need for novel and/or improved treatments for Alzheimer's disease due to the paucity of currently available therapies. As the life expectancy increases and the number of the elderly increases as well, the need for different treatments in management and treatment of AD patients is becoming more pronounced.

### SUMMARY OF THE INVENTION

Among the several aspects of the invention is a method and a composition for the treatment or prevention of Alzheimer's disease in a subject. The composition comprises a cyclooxygenase-2 selective inhibitor and an amyloid beta vaccine, and the method comprises administering to the subject a cyclooxygenase-2 selective inhibitor or a pharmaceutically acceptable salt or prodrug thereof and one or more doses of amyloid beta vaccines.

In one embodiment, the cyclooxygenase-2 selective inhibitor comprises a compound of the formula:

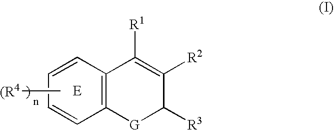

wherein n is an integer which is 0, 1, 2, 3 or 4;

wherein G is O, S or NR^{a};

wherein R^{a} is alkyl;

wherein R¹ is selected from the group consisting of H and aryl;

wherein R² is selected from the group consisting of carboxyl, aminocarbonyl, alkylsulfonylaminocarbonyl and alkoxycarbonyl;

wherein R³ is selected from the group consisting of haloalkyl, alkyl, aralkyl, cycloalkyl and aryl optionally substituted with one or more radicals selected from alkylthio, nitro and alkylsulfonyl; and

wherein each R⁴ is independently selected from the group consisting of H, halo, alkyl, aralkyl, alkoxy, aryloxy, heteroaryloxy, aralkyloxy, heteroaralkyloxy, haloalkyl, haloalkoxy, alkylamino, arylamino, aralkylamino, heteroarylamino, heteroarylalkylamino, nitro, amino, aminosulfonyl, alkylaminosulfonyl, arylaminosulfonyl, heteroarylaminosulfonyl, aralkylaminosulfonyl, heteroaralkylaminosulfonyl, heterocyclosulfonyl, alkylsulfonyl, hydroxyarylcarbonyl, nitroaryl, optionally substituted aryl, optionally substituted heteroaryl, aralkylcarbonyl, heteroarylcarbonyl, arylcarbonyl, aminocarbonyl, and alkylcarbonyl;

or wherein R⁴ together with the carbon atoms to which it is attached and the remainder of ring E forms a naphthyl radical;

or an isomer, a pharmaceutically acceptable salt, ester, or prodrug thereof.

In another embodiment, the cyclooxygenase-2 selective inhibitor or pharmaceutically acceptable salt or prodrug thereof comprises a compound of the formula:

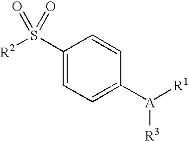

wherein A is selected from the group consisting of partially unsaturated or unsaturated heterocyclyl and partially unsaturated or unsaturated carbocyclic rings;

wherein R¹ is selected from the group consisting of heterocyclyl, cycloalkyl, cycloalkenyl and aryl, wherein R¹ is optionally substituted at a substitutable position with one or more radicals selected from alkyl, haloalkyl, cyano, carboxyl, alkoxycarbonyl, hydroxyl, hydroxyalkyl, haloalkoxy, amino, alkylamino, arylamino, nitro, alkoxyalkyl, alkylsulfinyl, halo, alkoxy and alkylthio;

wherein R² is selected from the group consisting of methyl or amino; and

wherein R³ is selected from the group consisting of a radical selected from H, halo, alkyl, alkenyl, alkynyl, oxo, cyano, carboxyl, cyanoalkyl, heterocyclyloxy, alkyloxy, alkylthio, alkylcarbonyl, cycloalkyl, aryl, haloalkyl, heterocyclyl, cycloalkenyl, aralkyl, heterocyclylalkyl, acyl, alkylthioalkyl, hydroxyalkyl, alkoxycarbonyl, arylcarbonyl, aralkylcarbonyl, aralkenyl, alkoxyalkyl, arylthioalkyl, aryloxyalkyl, aralkylthioalkyl, aralkoxyalkyl, alkoxyaralkoxyalkyl, alkoxycarbonylalkyl, aminocarbonyl, aminocarbonylalkyl, alkylaminocarbonyl, N-arylaminocarbonyl, N-alkyl-N-arylaminocarbonyl, alkylaminocarbonylalkyl, carboxyalkyl, alkylamino, N-arylamino, N-aralkylamino, N-alkyl-N-aralkylamino, N-alkyl-N-arylamino, aminoalkyl, alkylaminoalkyl, N-arylaminoalkyl, N-aralkylaminoalkyl, N-alkyl-N-aralkylaminoalkyl, N-alkyl-N-arylaminoalkyl, aryloxy, aralkoxy, arylthio, aralkylthio, alkylsulfinyl, alkylsulfonyl, aminosulfonyl, alkylaminosulfonyl, N-arylaminosulfonyl, arylsulfonyl, N-alkyl-N-arylaminosulfonyl.

In another embodiment, the amyloid beta vaccine comprises the amyloid peptide Abeta (1-43) or a fragment, variant or analog thereof. In another embodiment, the amyloid beta vaccine can be either monovalent or multivalent. In still another embodiment, the vaccine, in addition to at least one amyloid beta peptide or fragment thereof, comprises an adjuvant that contributes to the immunogenicity of the vaccine. Preferably, the adjuvant is selected from aluminum hydroxide or aluminum phosphate.

Other objects and features will be in part apparent and in part pointed out hereinafter.

Abbreviations and Definitions

The term “prevention” includes either preventing the onset of a clinically evident Alzheimer's disease or preventing the onset of a preclinically evident stage of Alzheimer's disease in a subject. This definition includes prophylactic treatment.

The terms “amyloid,” “amyloid plaque,” and “amyloid fibril” refer generally to insoluble proteinaceous substances with particular physical characteristics independent of the composition of proteins or other molecules that are found in the substance. Amyloid can be identified by its amorphous structure, eosinophilic staining, changes in thioflavin fluorescence, and homogeneous appearance. Protein or peptide components of amyloid are termed herein “amyloid polypeptides,” and as used herein refer to Abeta polypeptides and fragments thereof.

The term “β-amyloid peptide” or “Abeta” or “Aβ” as used herein refers to an approximately 4.2 kD protein which, in the brains of AD, Down's Syndrome, HCHWA-D (hereditary cerebral hemorrhage with amyloidosis of the Dutch type) and some normal aged subjects, forms the subunit of the amyloid filaments comprising the senile (amyloid) plaques and the amyloid deposits in small cerebral and meningeal blood vessels (amyloid angiopathy). Abeta peptide that is found in amyloid plaques generally exists in several isoforms that are about 39-43 amino acids long. Abeta can occur in a filamentous polymeric form (in this form, it exhibits the Congo-red and thioflavin-S dye-binding characteristics of amyloid), but it can also occur in a non-filamentous form (“preamyloid” or “amorphous” or “diffuse” deposits) in tissue, in which form no detectable birefringent staining by Congo red occurs. Abeta was first purified and a partial amino acid sequence reported in Glenner and Wong (Biochem. Biophys. Res. Commun., 120:885-890, 1984). The isolation procedure and the sequence data for the first 28 amino acids are described in, e.g., U.S. Pat. No. 4,666,829. The sequence of a 43-residue long A is disclosed, for example, in U.S. Pat. No. 6,284,221. As used herein, “Abeta” peptide includes fragments, analogs, and variants thereof.

The term “fragment” as used herein is intended to encompass a portion of an amyloid peptide described herein.

The term “variant” as used herein refers to a molecule substantially similar in structure and biological activity or immunological properties to either the entire molecule or a fragment thereof. Thus, provided that two molecules possess a similar activity, they are considered variants even if the sequence of their amino acid residues is not identical.

The term “analog” as used herein refers to a molecule substantially similar in function to either the entire molecule or to a fragment thereof. An analog may contain chemical moieties that are not normally a part of the molecule, but that may, for example, improve the molecule's half-life or decrease its toxicity. Moieties capable of mediating such effects are disclosed in Remington's Pharmaceutical Sciences (1980).

The phrase “therapeutically-effective” is intended to qualify the amount of each agent which will achieve the goal of improvement in disorder severity and the frequency of incidence over no treatment or treatment of each agent by itself, while avoiding adverse side effects typically associated with alternative therapies.

The term “treatment” includes alleviation, elimination of causation of or prevention of undesirable symptoms associated with Alzheimer's disease. Treatment as used herein includes prophylactic treatment.

The term “subject” for purposes of treatment includes any human or animal subject who is afflicted or predisposed to Alzheimer's disease. The subject can be a domestic livestock species, a laboratory animal species, a zoo animal or a companion animal. In one embodiment, the subject is a mammal. In a preferred embodiment, the mammal is a human being.

The term “cyclooxygenase-2 selective inhibitor” denotes a compound able to inhibit cyclooxygenase-2 without significant inhibition of cyclooxygenase-1. Preferably, it includes compounds that have a cyclooxygenase-2 IC₅₀ of less than about 0.2 micro molar, and also have a selectivity ratio of cyclooxygenase-2 inhibition over cyclooxygenase-1 inhibition of at least 50, and more preferably of at least 100. Even more preferably, the compounds have a cyclooxygenase-1 IC₅₀ of greater than about 1 micro molar, and more preferably of greater than 10 micro molar. Inhibitors of the cyclooxygenase pathway in the metabolism of arachidonic acid used in the present method may inhibit enzyme activity through a variety of mechanisms. By the way of example, and without limitation, the inhibitors used in the methods described herein may block the enzyme activity directly by acting as a substrate for the enzyme.

The term “hydrido” denotes a single hydrogen atom (H). This hydrido radical may be attached, for example, to an oxygen atom to form a hydroxyl radical or two hydrido radicals may be attached to a carbon atom to form a methylene (—CH2-) radical.

Where used, either alone or within other terms such as “haloalkyl”, “alkylsulfonyl”, “alkoxyalkyl” and “hydroxyalkyl”, the term “alkyl” embraces linear, cyclic or branched radicals having one to about twenty carbon atoms or, preferably, one to about twelve carbon atoms. More preferred alkyl radicals are “lower alkyl” radicals having one to about ten carbon atoms. Most preferred are lower alkyl radicals having one to about six carbon atoms. Examples of such radicals include methyl, ethyl, n-propyl, isopropyl, n-butyl, isobutyl, sec-butyl, tert-butyl, pentyl, iso-amyl, hexyl and the like.

The term “alkenyl” embraces linear or branched radicals having at least one carbon-carbon double bond of two to about twenty carbon atoms or, preferably, two to about twelve carbon atoms. More preferred alkyl radicals are “lower alkenyl” radicals having two to about six carbon atoms. Examples of alkenyl radicals include ethenyl, propenyl, allyl, propenyl, butenyl and 4-methylbutenyl.

The term “alkynyl” denotes linear or branched radicals having two to about twenty carbon atoms or, preferably, two to about twelve carbon atoms. More preferred alkynyl radicals are “lower alkynyl” radicals having two to about ten carbon atoms. Most preferred are lower alkynyl radicals having two to about six carbon atoms. Examples of such radicals include propargyl, butynyl, and the like.

The terms “alkenyl”, “lower alkenyl”, embrace radicals having “cis” and “trans” orientations, or alternatively, “E” and “Z” orientations. The term “cycloalkyl” embraces saturated carbocyclic radicals having three to twelve carbon atoms. More preferred cycloalkyl radicals are “lower cycloalkyl” radicals having three to about eight carbon atoms. Examples of such radicals include cyclopropyl, cyclobutyl, cyclopentyl and cyclohexyl.

The term “cycloalkenyl” embraces partially unsaturated carbocyclic radicals having three to twelve carbon atoms. More preferred cycloalkenyl radicals are “lower cycloalkenyl” radicals having four to about eight carbon atoms. Examples of such radicals include cyclobutenyl, cyclopentenyl, cyclopentadienyl, and cyclohexenyl.

The term “halo” means halogens such as fluorine, chlorine, bromine or iodine.

The term “haloalkyl” embraces radicals wherein any one or more of the alkyl carbon atoms is substituted with halo as defined above. Specifically embraced are monohaloalkyl, dihaloalkyl and polyhaloalkyl radicals. A monohaloalkyl radical, for one example, may have either an iodo, bromo, chloro or fluoro atom within the radical. Dihalo and polyhaloalkyl radicals may have two or more of the same halo atoms or a combination of different halo radicals. “Lower haloalkyl” embraces radicals having 1-6 carbon atoms. Examples of haloalkyl radicals include fluoromethyl, difluoromethyl, trifluoromethyl, chloromethyl, dichloromethyl, trichloromethyl, trichloromethyl, pentafluoroethyl, heptafluoropropyl, difluorochloromethyl, dichlorofluoromethyl, difluoroethyl, difluoropropyl, dichloroethyl and dichloropropyl.

The term “hydroxyalkyl” embraces linear or branched alkyl radicals having one to about ten carbon atoms any one of which may be substituted with one or more hydroxyl radicals. More preferred hydroxyalkyl radicals are “lower hydroxyalkyl” radicals having one to six carbon atoms and one or more hydroxyl radicals. Examples of such radicals include hydroxymethyl, hydroxyethyl, hydroxypropyl, hydroxybutyl and hydroxyhexyl.

The terms “alkoxy” and “alkyloxy” embrace linear or branched oxy-containing radicals each having alkyl portions of one to about ten carbon atoms. More preferred alkoxy radicals are “lower alkoxy” radicals having one to six carbon atoms. Examples of such radicals include methoxy, ethoxy, propoxy, butoxy and tert-butoxy.

The term “alkoxyalkyl” embraces alkyl radicals having one or more alkoxy radicals attached to the alkyl radical, that is, to form monoalkoxyalkyl and dialkoxyalkyl radicals. The “alkoxy” radicals may be further substituted with one or more halo atoms, such as fluoro, chloro or bromo, to provide haloalkoxy radicals. More preferred haloalkoxy radicals are “lower haloalkoxy” radicals having one to six carbon atoms and one or more halo radicals. Examples of such radicals include fluoromethoxy, chloromethoxy, trifluoromethoxy, trifluoroethoxy, fluoroethoxy and fluoropropoxy.

The term “aryl”, alone or in combination, means a carbocyclic aromatic system containing one, two or three rings wherein such rings may be attached together in a pendent manner or may be fused. The term “aryl” embraces aromatic radicals such as phenyl, naphthyl, tetrahydronaphthyl, indane and biphenyl. Aryl moieties may also be substituted at a substitutable position with one or more substituents selected independently from alkyl, alkoxyalkyl, alkylaminoalkyl, carboxyalkyl, alkoxycarbonylalkyl, aminocarbonylalkyl, alkoxy, aralkoxy, hydroxyl, amino, halo, nitro, alkylamino, acyl, cyano, carboxy, aminocarbonyl, alkoxycarbonyl and aralkoxycarbonyl.

The term “heterocyclyl” embraces saturated, partially unsaturated and unsaturated heteroatom-containing ring-shaped radicals, where the heteroatoms may be selected from nitrogen, sulfur and oxygen. Examples of saturated heterocyclyl radicals include saturated 3 to 6-membered heteromonocylic group containing 1 to 4 nitrogen atoms (e.g. pyrrolidinyl, imidazolidinyl, piperidino, piperazinyl, etc.); saturated 3 to 6-membered heteromonocyclic group containing 1 to 2 oxygen atoms and 1 to 3 nitrogen atoms (e.g. morpholinyl, etc.); saturated 3 to 6-membered heteromonocyclic group containing 1 to 2 sulfur atoms and 1 to 3 nitrogen atoms (e.g., thiazolidinyl, etc.). Examples of partially unsaturated heterocyclyl radicals include dihydrothiophene, dihydropyran, dihydrofuran and dihydrothiazole.

The term “heteroaryl” embraces unsaturated heterocyclyl radicals. Examples of unsaturated heterocyclyl radicals, also termed “heteroaryl” radicals include unsaturated 3 to 6 membered heteromonocyclic group containing 1 to 4 nitrogen atoms, for example, pyrrolyl, pyrrolinyl, imidazolyl, pyrazolyl, pyridyl, pyrimidyl, pyrazinyl, pyridazinyl, triazolyl (e.g., 4H-1,2,4-triazolyl, 1H-1,2,3-triazolyl, 2H-1,2,3-triazolyl, etc.) tetrazolyl (e.g. 1H-tetrazolyl, 2H-tetrazolyl, etc.), etc.; unsaturated condensed heterocyclyl group containing 1 to 5 nitrogen atoms, for example, indolyl, isoindolyl, indolizinyl, benzimidazolyl, quinolyl, isoquinolyl, indazolyl, benzotriazolyl, tetrazolopyridazinyl (e.g., tetrazolo[1,5-b]pyridazinyl, etc.), etc.; unsaturated 3 to 6-membered heteromonocyclic group containing an oxygen atom, for example, pyranyl, furyl, etc.; unsaturated 3 to 6-membered heteromonocyclic group containing a sulfur atom, for example, thienyl, etc.; unsaturated 3- to 6-membered heteromonocyclic group containing 1 to 2 oxygen atoms and 1 to 3 nitrogen atoms, for example, oxazolyl, isoxazolyl, oxadiazolyl (e.g., 1,2,4-oxadiazolyl, 1,3,4-oxadiazolyl, 1,2,5-oxadiazolyl, etc.) etc.; unsaturated condensed heterocyclyl group containing 1 to 2 oxygen atoms and 1 to 3 nitrogen atoms (e.g. benzoxazolyl, benzoxadiazolyl, etc.); unsaturated 3 to 6-membered heteromonocyclic group containing 1 to 2 sulfur atoms and 1 to 3 nitrogen atoms, for example, thiazolyl, thiadiazolyl (e.g., 1,2,4-thiadiazolyl, 1,3,4-thiadiazolyl, 1,2,5-thiadiazolyl, etc.) etc.; unsaturated condensed heterocyclyl group containing 1 to 2 sulfur atoms and 1 to 3 nitrogen atoms (e.g., benzothiazolyl, benzothiadiazolyl, etc.) and the like. The term also embraces radicals where heterocyclyl radicals are fused with aryl radicals. Examples of such fused bicyclic radicals include benzofuran, benzothiophene, and the like. Said “heterocyclyl group” may have 1 to 3 substituents such as alkyl, hydroxyl, halo, alkoxy, oxo, amino and alkylamino.

The term “alkylthio” embraces radicals containing a linear or branched alkyl radical, of one to about ten carbon atoms attached to a divalent sulfur atom. More preferred alkylthio radicals are “lower alkylthio” radicals having alkyl radicals of one to six carbon atoms. Examples of such lower alkylthio radicals are methylthio, ethylthio, propylthio, butylthio and hexylthio.

The term “alkylthioalkyl” embraces radicals containing an alkylthio radical attached through the divalent sulfur atom to an alkyl radical of one to about ten carbon atoms. More preferred alkylthioalkyl radicals are “lower alkylthioalkyl” radicals having alkyl radicals of one to six carbon atoms. Examples of such lower alkylthioalkyl radicals include methylthiomethyl.

The term “alkylsulfinyl” embraces radicals containing a linear or branched alkyl radical, of one to ten carbon atoms, attached to a divalent —S(═O)— radical. More preferred alkylsulfinyl radicals are “lower alkylsulfinyl” radicals having alkyl radicals of one to six carbon atoms. Examples of such lower alkylsulfinyl radicals include methylsulfinyl, ethylsulfinyl, butylsulfinyl and hexylsulfinyl.

The term “sulfonyl”, whether used alone or linked to other terms such as alkylsulfonyl, denotes respectively divalent radicals —SO2—. “Alkylsulfonyl” embraces alkyl radicals attached to a sulfonyl radical, where alkyl is defined as above. More preferred alkylsulfonyl radicals are “lower alkylsulfonyl” radicals having one to six carbon atoms. Examples of such lower alkylsulfonyl radicals include methylsulfonyl, ethylsulfonyl and propylsulfonyl. The “alkylsulfonyl” radicals may be further substituted with one or more halo atoms, such as fluoro, chloro or bromo, to provide haloalkylsulfonyl radicals. The terms “sulfamyl”, “aminosulfonyl” and “sulfonamidyl” denote NH2O2S—.

The term “acyl” denotes a radical provided by the residue after removal of hydroxyl from an organic acid. Examples of such acyl radicals include alkanoyl and aroyl radicals. Examples of such lower alkanoyl radicals include formyl, acetyl, propionyl, butyryl, isobutyryl, valeryl, isovaleryl, pivaloyl, hexanoyl, trifluoroacetyl.

The term “carbonyl”, whether used alone or with other terms, such as “alkoxycarbonyl”, denotes —(C═O)—.

The term “aroyl” embraces aryl radicals with a carbonyl radical as defined above. Examples of aroyl include benzoyl, naphthoyl, and the like and the aryl in said aroyl may be additionally substituted.

The terms “carboxy” or “carboxyl”, whether used alone or with other terms, such as “carboxyalkyl”, denotes —CO2H.

The term “carboxyalkyl” embraces alkyl radicals substituted with a carboxy radical. More preferred are “lower carboxyalkyl” which embrace lower alkyl radicals as defined above, and may be additionally substituted on the alkyl radical with halo. Examples of such lower carboxyalkyl radicals include carboxymethyl, carboxyethyl and carboxypropyl.

The term “alkoxycarbonyl” means a radical containing an alkoxy radical, as defined above, attached via an oxygen atom to a carbonyl radical. More preferred are “lower alkoxycarbonyl” radicals with alkyl portions having 1 to 6 carbons. Examples of such lower alkoxycarbonyl (ester) radicals include substituted or unsubstituted methoxycarbonyl, ethoxycarbonyl, propoxycarbonyl, butoxycarbonyl and hexyloxycarbonyl.

The terms “alkylcarbonyl”, “arylcarbonyl” and “aralkylcarbonyl” include radicals having alkyl, aryl and aralkyl radicals, as defined above, attached to a carbonyl radical. Examples of such radicals include substituted or unsubstituted methylcarbonyl, ethylcarbonyl, phenylcarbonyl and benzylcarbonyl.

The term “aralkyl” embraces aryl-substituted alkyl radicals such as benzyl, diphenylmethyl, triphenylmethyl, phenylethyl, and diphenylethyl. The aryl in said aralkyl may be additionally substituted with halo, alkyl, alkoxy, halkoalkyl and haloalkoxy. The terms benzyl and phenylmethyl are interchangeable.

The term “heterocyclylalkyl” embraces saturated and partially unsaturated heterocyclyl-substituted alkyl radicals, such as pyrrolidinylmethyl, and heteroaryl-substituted alkyl radicals, such as pyridylmethyl, quinolylmethyl, thienylmethyl, furylethyl, and quinolylethyl. The heteroaryl in said heteroaralkyl may be additionally substituted with halo, alkyl, alkoxy, halkoalkyl and haloalkoxy.

The term “aralkoxy” embraces aralkyl radicals attached through an oxygen atom to other radicals.

The term “aralkoxyalkyl” embraces aralkoxy radicals attached through an oxygen atom to an alkyl radical.

The term “aralkylthio” embraces aralkyl radicals attached to a sulfur atom.

The term “aralkylthioalkyl” embraces aralkylthio radicals attached through a sulfur atom to an alkyl radical.

The term “aminoalkyl” embraces alkyl radicals substituted with one or more amino radicals. More preferred are “lower aminoalkyl” radicals. Examples of such radicals include aminomethyl, aminoethyl, and the like.

The term “alkylamino” denotes amino groups that have been substituted with one or two alkyl radicals. Preferred are “lower N-alkylamino” radicals having alkyl portions having 1 to 6 carbon atoms. Suitable lower alkylamino may be mono or dialkylamino such as N-methylamino, N-ethylamino, N,N-dimethylamino, N,N-diethylamino or the like.

The term “arylamino” denotes amino groups, which have been substituted with one or two aryl radicals, such as N-phenylamino. The “arylamino” radicals may be further substituted on the aryl ring portion of the radical.

The term “aralkylamino” embraces aralkyl radicals attached through an amino nitrogen atom to other radicals. The terms “N-arylaminoalkyl” and “N-aryl-N-alkyl-aminoalkyl” denote amino groups which have been substituted with one aryl radical or one aryl and one alkyl radical, respectively, and having the amino group attached to an alkyl radical. Examples of such radicals include N-phenylaminomethyl and N-phenyl-N-methylaminomethyl.

The term “aminocarbonyl” denotes an amide group of the formula —C(═O)NH2.

The term “alkylaminocarbonyl” denotes an aminocarbonyl group that has been substituted with one or two alkyl radicals on the amino nitrogen atom. Preferred are “N-alkylaminocarbonyl” “N,N-dialkylaminocarbonyl” radicals. More preferred are “lower N-alkylaminocarbonyl” “lower N,N-dialkylaminocarbonyl” radicals with lower alkyl portions as defined above.

The term “alkylaminoalkyl” embraces radicals having one or more alkyl radicals attached to an aminoalkyl radical.

The term “aryloxyalkyl” embraces radicals having an aryl radical attached to an alkyl radical through a divalent oxygen atom.

The term “arylthioalkyl” embraces radicals having an aryl radical attached to an alkyl radical through a divalent sulfur atom.

### DESCRIPTION OF THE PREFERRED EMBODIMENTS

The present invention provides a combination therapy comprising the administration to a subject of a therapeutically effective amount of a COX-2 selective inhibitor in combination with amyloid vaccination. The combination therapy is used to treat or prevent Alzheimer's disease. When administered as part of a combination therapy, the COX-2 selective inhibitor together with the amyloid beta vaccine provides enhanced treatment options as compared to administration of either the amyloid beta vaccine or the COX-2 selective inhibitor alone.

Cox-2 Selective Inhibitors

Any cyclooxygenase-2 selective inhibitor or prodrug or pharmaceutically acceptable salt thereof may be employed in the composition of the current invention. In one embodiment, the cyclooxygenase-2 selective inhibitor can be, for example, the cyclooxygenase-2 selective inhibitor meloxicam, Formula B-1 (CAS registry number 71125-38-7) or a pharmaceutically acceptable salt or prodrug thereof.

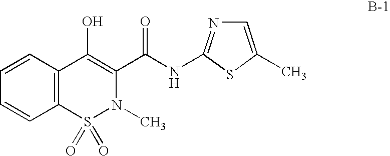

In yet another embodiment, the cyclooxygenase-2 selective inhibitor is the cyclooxygenase-2 selective inhibitor, 6-[[5-(4-chlorobenzoyl)-1,4-dimethyl-1H-pyrrol-2-yl]methyl]-3(2H)-pyridazinone, Formula B-2 (CAS registry number 179382-91-3) or a pharmaceutically acceptable salt or prodrug thereof.

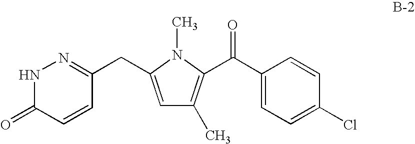

In a preferred embodiment the cyclooxygenase-2 selective inhibitor is preferably of the chromene structural class that is a substituted benzopyran or a substituted benzopyran analog, and even more preferably selected from the group consisting of substituted benzothiopyrans, dihydroquinolines, or dihydronaphthalenes having the general Formula I shown below and possessing, by way of example and not limitation, the structures disclosed in Table 1, including the diastereomers, enantiomers, racemates, tautomers, salts, esters, amides and prodrugs thereof. Furthermore, benzopyran cyclooxygenase-2 selective inhibitors useful in the practice of the present methods are described in U.S. Pat. Nos. 6,034,256 and 6,077,850 herein incorporated by reference in their entirety.

In one embodiment, the cyclooxygenase-2 selective inhibitor is of the chromene structural class and is represented by Formula I:

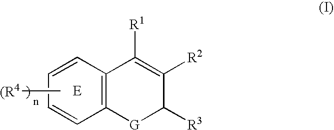

or an isomer, a pharmaceutically acceptable salt, ester, or prodrug thereof;

wherein n is an integer which is 0, 1, 2, 3 or 4;

wherein G is O, S or NR^{a};

wherein R^{a} is alkyl;

wherein R¹ is selected from the group consisting of H and aryl;

wherein R² is selected from the group consisting of carboxyl, aminocarbonyl, alkylsulfonylaminocarbonyl and alkoxycarbonyl;

wherein R³ is selected from the group consisting of haloalkyl, alkyl, aralkyl, cycloalkyl and aryl optionally substituted with one or more radicals selected from alkylthio, nitro and alkylsulfonyl; and

wherein each R⁴ is independently selected from the group consisting of H, halo, alkyl, aralkyl, alkoxy, aryloxy, heteroaryloxy, aralkyloxy, heteroaralkyloxy, haloalkyl, haloalkoxy, alkylamino, arylamino, aralkylamino, heteroarylamino, heteroarylalkylamino, nitro, amino, aminosulfonyl, alkylaminosulfonyl, arylaminosulfonyl, heteroarylaminosulfonyl, aralkylaminosulfonyl, heteroaralkylaminosulfonyl, heterocyclosulfonyl, alkylsulfonyl, hydroxyarylcarbonyl, nitroaryl, optionally substituted aryl, optionally substituted heteroaryl, aralkylcarbonyl, heteroarylcarbonyl, arylcarbonyl, aminocarbonyl, and alkylcarbonyl;

or wherein R⁴ together with the carbon atoms to which it is attached and the remainder of ring E forms a naphthyl radical.

The cyclooxygenase-2 selective inhibitor may also be a compound of Formula (I) or an isomer, a pharmaceutically acceptable salt, ester, or prodrug thereof wherein:

n is an integer which is 0, 1, 2, 3 or 4;

G is O, S or NR^{b};

R¹ is H;

R^{b} is alkyl;

R² is selected from the group consisting of carboxyl, aminocarbonyl, alkylsulfonylaminocarbonyl and alkoxycarbonyl;

R³ is selected from the group consisting of haloalkyl, alkyl, aralkyl, cycloalkyl and aryl, wherein haloalkyl, alkyl, aralkyl, cycloalkyl, and aryl each is independently optionally substituted with one or more radicals selected from the group consisting of alkylthio, nitro and alkylsulfonyl; and

each R⁴ is independently selected from the group consisting of hydrido, halo, alkyl, aralkyl, alkoxy, aryloxy, heteroaryloxy, aralkyloxy, heteroaralkyloxy, haloalkyl, haloalkoxy, alkylamino, arylamino, aralkylamino, heteroarylamino, heteroarylalkylamino, nitro, amino, aminosulfonyl, alkylaminosulfonyl, arylaminosulfonyl, heteroarylaminosulfonyl, aralkylaminosulfonyl, heteroaralkylaminosulfonyl, heterocyclosulfonyl, alkylsulfonyl, optionally substituted aryl, optionally substituted heteroaryl, aralkylcarbonyl, heteroarylcarbonyl, arylcarbonyl, aminocarbonyl, and alkylcarbonyl; or wherein R⁴ together with ring E forms a naphthyl radical.

In a further embodiment, the cyclooxygenase-2 selective inhibitor may also be a compound of Formula (I), or an isomer, a pharmaceutically acceptable salt, ester, or prodrug thereof; wherein:

n is an integer which is 0, 1, 2, 3 or 4;

G is oxygen or sulfur;

R¹ is H;

R² is carboxyl, lower alkyl, lower aralkyl or lower alkoxycarbonyl;

R³ is lower haloalkyl, lower cycloalkyl or phenyl; and

each R⁴ is H, halo, lower alkyl, lower alkoxy, lower haloalkyl, lower haloalkoxy, lower alkylamino, nitro, amino, aminosulfonyl, lower alkylaminosulfonyl, 5-membered heteroarylalkylaminosulfonyl, 6-membered heteroarylalkylaminosulfonyl, lower aralkylaminosulfonyl, 5-membered nitrogen-containing heterocyclosulfonyl, 6-membered-nitrogen containing heterocyclosulfonyl, lower alkylsulfonyl, optionally substituted phenyl, lower aralkylcarbonyl, or lower alkylcarbonyl; or

wherein R⁴ together with the carbon atoms to which it is attached and the remainder of ring E forms a naphthyl radical.

The cyclooxygenase-2 selective inhibitor may also be a compound of Formula (I) or an isomer, a pharmaceutically acceptable salt, ester, or prodrug thereof; wherein:

R² is carboxyl;

R³ is lower haloalkyl; and

each R⁴ is H, halo, lower alkyl, lower haloalkyl, lower haloalkoxy, lower alkylamino, amino, aminosulfonyl, lower alkylaminosulfonyl, 5-membered heteroarylalkylaminosulfonyl, 6-membered heteroarylalkylaminosulfonyl, lower aralkylaminosulfonyl, lower alkylsulfonyl, 6-membered nitrogen-containing heterocyclosulfonyl, optionally substituted phenyl, lower aralkylcarbonyl, or lower alkylcarbonyl; or wherein R⁴ together with ring E forms a naphthyl radical.

The cyclooxygenase-2 selective inhibitor may also be a compound of Formula (I) or an isomer, a pharmaceutically acceptable salt, ester, or prodrug thereof; wherein:

n is an integer which is 0, 1, 2, 3 or 4;

R³ is fluoromethyl, chloromethyl, dichloromethyl, trichloromethyl, pentafluoroethyl, heptafluoropropyl, difluoroethyl, difluoropropyl, dichloroethyl, dichloropropyl, difluoromethyl, or trifluoromethyl; and

each R⁴ is H, chloro, fluoro, bromo, iodo, methyl, ethyl, isopropyl, tert-butyl, butyl, isobutyl, pentyl, hexyl, methoxy, ethoxy, isopropyloxy, tertbutyloxy, trifluoromethyl, difluoromethyl, trifluoromethoxy, amino, N,N-dimethylamino, N,N-diethylamino, N-phenylmethylaminosulfonyl, N-phenylethylaminosulfonyl, N-(2-furylmethyl)aminosulfonyl, nitro, N,N-dimethylaminosulfonyl, aminosulfonyl, N-methylaminosulfonyl, N-ethylsulfonyl, 2,2-dimethylethylaminosulfonyl, N,N-dimethylaminosulfonyl, N-(2-methylpropyl)aminosulfonyl, N-morpholinosulfonyl, methylsulfonyl, benzylcarbonyl, 2,2-dimethylpropylcarbonyl, phenylacetyl or phenyl; or wherein R⁴ together with the carbon atoms to which it is attached and the remainder of ring E forms a naphthyl radical.

The cyclooxygenase-2 selective inhibitor may also be a compound of Formula (I) or an isomer, a pharmaceutically acceptable salt, ester, or prodrug thereof; wherein:

n is an integer which is 0, 1, 2, 3 or 4;

R³ is trifluoromethyl or pentafluoroethyl; and

each R⁴ is independently H, chloro, fluoro, bromo, iodo, methyl, ethyl, isopropyl, tert-butyl, methoxy, trifluoromethyl, trifluoromethoxy, N-phenylmethylaminosulfonyl, N-phenylethylaminosulfonyl, N-(2-furylmethyl)aminosulfonyl, N,N-dimethylaminosulfonyl, N-methylaminosulfonyl, N-(2,2-dimethylethyl)aminosulfonyl, dimethylaminosulfonyl, 2-methylpropylaminosulfonyl, N-morpholinosulfonyl, methylsulfonyl, benzylcarbonyl, or phenyl; or wherein R⁴ together with the carbon atoms to which it is attached and the remainder of ring E forms a naphthyl radical.

In yet another embodiment, the cyclooxygenase-2 selective inhibitor used in connection with the method(s) of the present invention can also be a compound having the structure of Formula (I) or an isomer, a pharmaceutically acceptable salt, ester, or prodrug thereof:

wherein:

n=4;

G is O or S;

R¹ is H;

R² is CO₂H;

R³ is lower haloalkyl;

a first R⁴ corresponding to R⁹ is hydrido or halo;

a second R⁴ corresponding to R¹⁰ is H, halo, lower alkyl, lower haloalkoxy, lower alkoxy, lower aralkylcarbonyl, lower dialkylaminosulfonyl, lower alkylaminosulfonyl, lower aralkylaminosulfonyl, lower heteroaralkylaminosulfonyl, 5-membered nitrogen-containing heterocyclosulfonyl, or 6-membered nitrogen-containing heterocyclosulfonyl;

a third R⁴ corresponding to R” is H, lower alkyl, halo, lower alkoxy, or aryl; and

a fourth R⁴ corresponding to R¹² is H, halo, lower alkyl, lower alkoxy, and aryl;

wherein Formula (I) is represented by Formula (Ia):

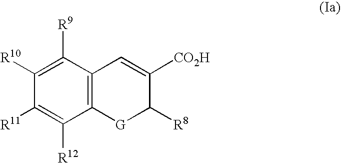

or an isomer, a pharmaceutically acceptable salt, ester, or prodrug thereof.

The cyclooxygenase-2 selective inhibitor used in connection with the method(s) of the present invention can also be a compound of having the structure of Formula (Ia) or an isomer, a pharmaceutically acceptable salt, ester, or prodrug thereof; wherein:

R⁸ is trifluoromethyl or pentafluoroethyl;

R⁹ is H, chloro, or fluoro;

R¹⁰ is H, chloro, bromo, fluoro, iodo, methyl, tert-butyl, trifluoromethoxy, methoxy, benzylcarbonyl, dimethylaminosulfonyl, isopropylaminosulfonyl, methylaminosulfonyl, benzylaminosulfonyl, phenylethylaminosulfonyl, methylpropylaminosulfonyl, methylsulfonyl, or morpholinosulfonyl;

R¹¹ is H, methyl, ethyl, isopropyl, tert-butyl, chloro, methoxy, diethylamino, or phenyl; and

R¹² is H, chloro, bromo, fluoro, methyl, ethyl, tert-butyl, methoxy, or phenyl.

Examples of exemplary chromene cyclooxygenase-2 selective inhibitors are depicted in Table 1 below.

TABLE 1
Examples of Chromene Cyclooxygenase-2 Selective
Inhibitors as Embodiments

| Compound | |
|---|---|
| Number | Structural Formula |
| B-3 | 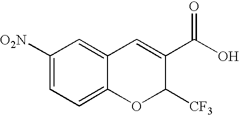 |
| | 6-Nitro-2-trifluoromethyl-2H-1- |
| | benzopyran-3-carboxylic acid |
| B-4 | 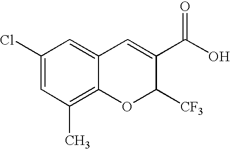 |
| | 6-Chloro-8-methyl-2-trifluoromethyl- |
| | 2H-1-benzopyran-3-carboxylic acid |
| B-5 | 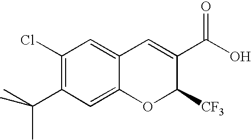 |
| | ((S)-6-Chloro-7-(1,1-dimethylethyl)-2- |
| | (trifluoromethyl-2H-1-benzopyran-3-carboxylic acid |
| B-6 | 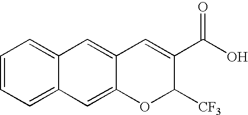 |
| | 2-Trifluoromethyl-2H-naphtho[2,3-b] |
| | pyran-3-carboxylic acid |
| B-7 | 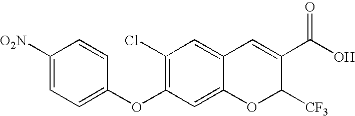 |
| | 6-Chloro-7-(4-nitrophenoxy)-2-(trifluoromethyl)-2H-1- |
| | benzopyran-3-carboxylic acid |
| B-8 | 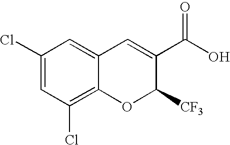 |
| | ((S)-6,8-Dichloro-2-(trifluoromethyl)- |
| | 2H-1-benzopyran-3-carboxylic acid |
| B-9 | 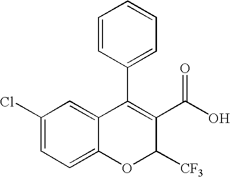 |
| | 6-Chloro-2-(trifluoromethyl)-4-phenyl-2H- |
| | 1-benzopyran-3-carboxylic acid |
| B-10 | 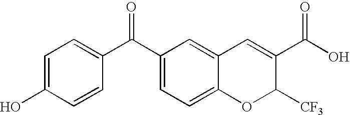 |
| | 6-(4-Hydroxybenzoyl)-2-(trifluoromethyl)- |
| | 2H-1-benzopyran-3-carboxylic acid |
| B-11 | 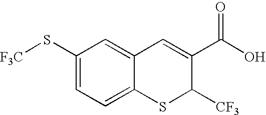 |
| | 2-(Trifluoromethyl)-6-[(trifluoromethyl)thio]- |
| | 2H-1-benzothiopyran-3-carboxylic acid |
| B-12 | 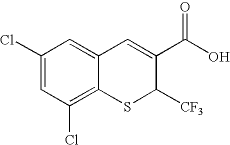 |
| | 6,8-Dichloro-2-trifluoromethyl-2H-1- |
| | benzothiopyran-3-carboxylic acid |
| B-13 | 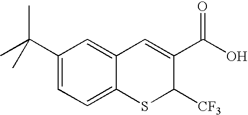 |
| | 6-(1,1-Dimethylethyl)-2-(trifluoromethyl)- |
| | 2H-1-benzothiopyran-3-carboxylic acid |
| B-14 | 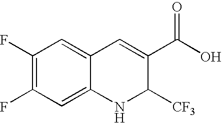 |
| | 6,7-Difluoro-1,2-dihydro-2-(trifluoromethyl)- |
| | 3-quinolinecarboxylic acid |
| B-15 | 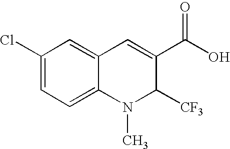 |
| | 6-Chloro-1,2-dihydro-1-methyl-2-(trifluoromethyl)- |
| | 3-quinolinecarboxylic acid |
| B-16 | 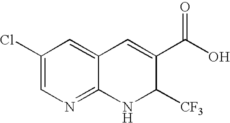 |
| | 6-Chloro-2-(trifluoromethyl)-1,2-dihydro |
| | [1,8]naphthyridine-3-carboxylic acid |
| B-17 | 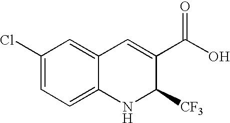 |
| | ((S)-6-Chloro-1,2-dihydro-2-(trifluoromethyl)- |
| | 3-quinolinecarboxylic acid |

In a further preferred embodiment, the cyclooxygenase inhibitor is selected from the class of tricyclic cyclooxygenase-2 selective inhibitors represented by the general structure of Formula II:

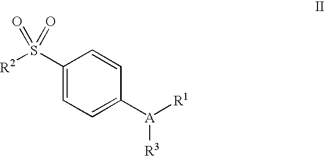

wherein A is selected from the group consisting of partially unsaturated or unsaturated heterocyclyl and partially unsaturated or unsaturated carbocyclic rings;

wherein R¹ is selected from the group consisting of heterocyclyl, cycloalkyl, cycloalkenyl and aryl, wherein R¹ is optionally substituted at a substitutable position with one or more radicals selected from alkyl, haloalkyl, cyano, carboxyl, alkoxycarbonyl, hydroxyl, hydroxyalkyl, haloalkoxy, amino, alkylamino, arylamino, nitro, alkoxyalkyl, alkylsulfinyl, halo, alkoxy and alkylthio;

wherein R² is selected from the group consisting of methyl or amino; and

wherein R³ is selected from the group consisting of a radical selected from H, halo, alkyl, alkenyl, alkynyl, oxo, cyano, carboxyl, cyanoalkyl, heterocyclyloxy, alkyloxy, alkylthio, alkylcarbonyl, cycloalkyl, aryl, haloalkyl, heterocyclyl, cycloalkenyl, aralkyl, heterocyclylalkyl, acyl, alkylthioalkyl, hydroxyalkyl, alkoxycarbonyl, arylcarbonyl, aralkylcarbonyl, aralkenyl, alkoxyalkyl, arylthioalkyl, aryloxyalkyl, aralkylthioalkyl, aralkoxyalkyl, alkoxyaralkoxyalkyl, alkoxycarbonylalkyl, aminocarbonyl, aminocarbonylalkyl, alkylaminocarbonyl, N-arylaminocarbonyl, N-alkyl-N-arylaminocarbonyl, alkylaminocarbonylalkyl, carboxyalkyl, alkylamino, N-arylamino, N-aralkylamino, N-alkyl-N-aralkylamino, N-alkyl-N-arylamino, aminoalkyl, alkylaminoalkyl, N-arylaminoalkyl, N-aralkylaminoalkyl, N-alkyl-N-aralkylaminoalkyl, N-alkyl-N-arylaminoalkyl, aryloxy, aralkoxy, arylthio, aralkylthio, alkylsulfinyl, alkylsulfonyl, aminosulfonyl, alkylaminosulfonyl, N-arylaminosulfonyl, arylsulfonyl, N-alkyl-N-arylaminosulfonyl; or a pharmaceutically acceptable salt thereof.

In a still more preferred embodiment of the invention the cyclooxygenase-2 selective inhibitor represented by the above Formula II is selected from the group of compounds, illustrated in Table 2, consisting of celecoxib (B-18; U.S. Pat. No. 5,466,823; CAS No. 169590-42-5), valdecoxib (B-19; U.S. Pat. No. 5,633,272; CAS No. 181695-72-7), deracoxib (B-20; U.S. Pat. No. 5,521,207; CAS No. 169590-41-4), rofecoxib (B-21; CAS No. 162011-90-7), etoricoxib (MK-663; B-22; PCT publication WO 98/03484), JTE-522 (B-23), or an isomer, ester, a pharmaceutically acceptable salt or prodrug thereof.

TABLE 2
Examples of Tricyclic Cyclooxygenase-2 Selective
Inhibitors as Embodiments

| Compound | |
|---|---|
| Number | Structural Formula |
| B-18 | 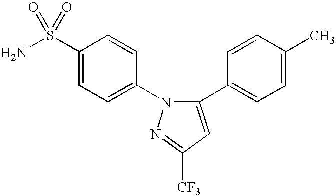 |
| B-19 | 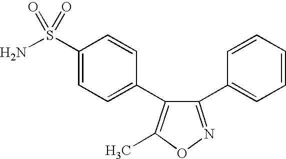 |
| B-20 | 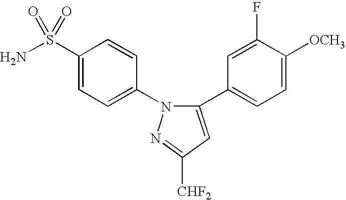 |
| B-21 | 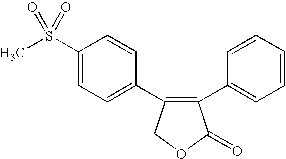 |
| B-22 | 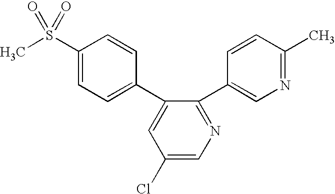 |
| B-23 | 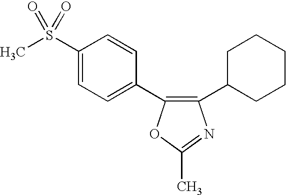 |

In an even more preferred embodiment, the cyclooxygenase-2 selective inhibitor is selected from the group consisting of celecoxib, rofecoxib and etoricoxib.

In another highly preferred embodiment of the invention, parecoxib (B-24, U.S. Pat. No. 5,932,598, CAS No. 198470-84-7), which is a therapeutically effective prodrug of the tricyclic cyclooxygenase-2 selective inhibitor valdecoxib, B-19, may be advantageously employed as a source of a cyclooxygenase inhibitor (U.S. Pat. No. 5,932,598, herein incorporated by reference).

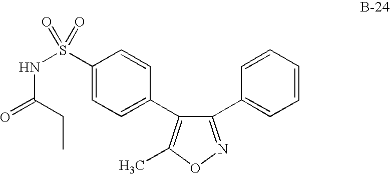

A preferred form of parecoxib is sodium parecoxib.

In another preferred embodiment of the invention, the compound having the formula B-25 that has been previously described in International Publication number WO 00/24719 (which is herein incorporated by reference), is another tricyclic cyclooxygenase-2 selective inhibitor which may be advantageously employed.

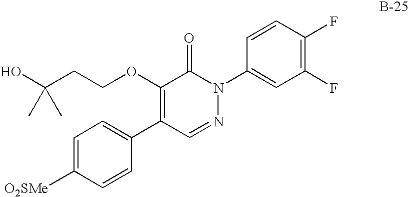

Another preferred cyclooxygenase-2 selective inhibitor that is useful in connection with the method(s) of the present invention is N-(2-cyclohexyloxynitrophenyl)-methane sulfonamide (NS-398) having a structure shown below as B-26.

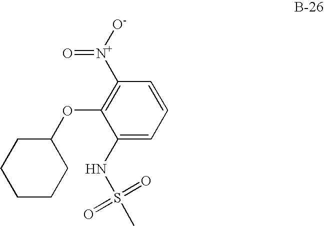

In yet a further preferred embodiment of the invention, the cyclooxygenase inhibitor used in connection with the method(s) of the present invention can be selected from the class of phenylacetic acid derivative cyclooxygenase-2 selective inhibitors represented by the general structure of Formula (III):

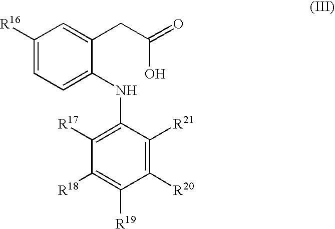

or an isomer, a pharmaceutically acceptable salt, ester, or prodrug thereof;

wherein

R¹⁶ is methyl or ethyl;

R¹⁷ is chloro or fluoro;

R¹⁸ is hydrogen or fluoro;

R¹⁹ is hydrogen, fluoro, chloro, methyl, ethyl, methoxy, ethoxy or hydroxy;

R²⁰ is hydrogen or fluoro; and

R²¹ is chloro, fluoro, trifluoromethyl or methyl, provided that R¹⁷, R¹⁸, R¹⁹ and R²⁰ are not all fluoro when R¹⁶ is ethyl and R¹⁹ is H.

A particularly preferred phenylacetic acid derivative cyclooxygenase-2 selective inhibitor used in connection with the method(s) of the present invention is a compound that has the designation of COX 189 (B-211) and that has the structure shown in Formula (III) or an isomer, a pharmaceutically acceptable salt, ester, or prodrug thereof, wherein:

R¹⁶ is ethyl;

R¹⁷ and R¹⁹ are chloro;

R¹⁸ and R²⁰ are hydrogen; and and R²¹ is methyl.

In yet another embodiment, the cyclooxygenase-2 selective inhibitor is represented by Formula (IV):

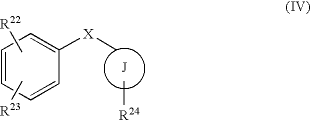

or an isomer, a pharmaceutically acceptable salt, an ester, or a prodrug thereof,

wherein:

X is O or S;

J is a carbocycle or a heterocycle;

R²² is NHSO₂CH₃ or F;

R²³ is H, NO₂, or F; and

R²⁴ is H, NHSO₂CH₃, or (SO₂CH₃)C₆H₄.

According to another embodiment, the cyclooxygenase-2 selective inhibitors used in the present method(s) have the structural Formula (V):

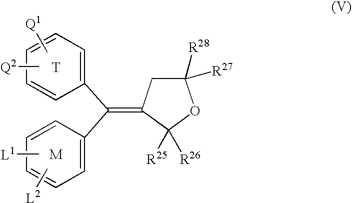

or an isomer, a pharmaceutically acceptable salt, an ester, or a prodrug thereof, wherein:

T and M independently are phenyl, naphthyl, a radical derived from a heterocycle comprising 5 to 6 members and possessing from 1 to 4 heteroatoms, or a radical derived from a saturated hydrocarbon ring having from 3 to 7 carbon atoms;

Q¹, Q², L¹ or L² are independently hydrogen, halogen, lower alkyl having from 1 to 6 carbon atoms, trifluoromethyl, or lower methoxy having from 1 to 6 carbon atoms; and

at least one of Q¹, Q², L¹ or L² is in the para position and is —S(O)ₙ—R, wherein n is 0, 1, or 2 and R is a lower alkyl radical having 1 to 6 carbon atoms or a lower haloalkyl radical having from 1 to 6 carbon atoms, or an —SO₂NH₂; or,

Q¹ and Q² are methylenedioxy; or

L¹ and L² are methylenedioxy; and

R²⁵, R²⁶, R²⁷, and R²⁸ are independently hydrogen, halogen, lower alkyl radical having from 1 to 6 carbon atoms, lower haloalkyl radical having from 1 to 6 carbon atoms, or an aromatic radical selected from the group consisting of phenyl, naphthyl, thienyl, furyl and pyridyl; or,

R²⁵ and R²⁶ are 0; or,

R²⁷ and R²⁸ are 0; or,

R²⁵, R²⁶, together with the carbon atom to which they are attached, form a saturated hydrocarbon ring having from 3 to 7 carbon atoms; or,

R²⁷, R²⁸, together with the carbon atom to which they are attached, form a saturated hydrocarbon ring having from 3 to 7 carbon atoms.

In a particularly preferred embodiment, the compounds N-(2-cyclohexyloxynitrophenyl)methane sulfonamide, and (E)-4-[(4-methylphenyl)(tetrahydro-2-oxo-3-furanylidene) methyl]benzenesulfonamide having the structure of Formula (V) are employed as cyclooxygenase-2 selective inhibitors.

Exemplary compounds that are useful for the cyclooxygenase-2 selective inhibitor in connection with the method(s) of the present invention, the structures for which are set forth in Table 3 below, include, but are not limited to:

6-chloro-2-trifluoromethyl-2H-1-benzopyran-3-carboxylic acid (B-27);

6-chloro-7-methyl-2-trifluoromethyl-2H-1-benzopyran-3-carboxylic acid (B-28);

8-(1-methylethyl)-2-trifluoromethyl-2H-1-benzopyran-3-carboxylic acid (B-29);

6-chloro-8-(1-methylethyl)-2-trifluoromethyl-2H-1-benzopyran-3-carboxylic acid (B-30);

2-trifluoromethyl-3H-naphtho[2,1-b]pyran-3-carboxylic acid (B-31);

7-(1,1-dimethylethyl)-2-trifluoromethyl-2H-1-benzopyran-3-carboxylic acid (B-32);

6-bromo-2-trifluoromethyl-2H-1-benzopyran-3-carboxylic acid (B-33);

8-chloro-2-trifluoromethyl-2H-1-benzopyran-3-carboxylic acid (B-34);

6-trifluoromethoxy-2-trifluoromethyl-2H-1-benzopyran-3-carboxylic acid (B-35);

5,7-dichloro-2-trifluoromethyl-2H-1-benzopyran-3-carboxylic acid (B-36);

8-phenyl-2-trifluoromethyl-2H-1-benzopyran-3-carboxylic acid (B-37);

7,8-dimethyl-2-trifluoromethyl-2H-1-benzopyran-3-carboxylic acid (B-38);

6,8-bis(dimethylethyl)-2-trifluoromethyl-2H-1-benzopyran-3-carboxylic acid (B-39);

7-(1-methylethyl)-2-trifluoromethyl-2H-1-benzopyran-3-carboxylic acid (B-40);

7-phenyl-2-trifluoromethyl-2H-1-benzopyran-3-carboxylic acid (B-41);

6-chloro-7-ethyl-2-trifluoromethyl-2H-1-benzopyran-3-carboxylic acid (B-42);

6-chloro-8-ethyl-2-trifluoromethyl-2H-1-benzopyran-3-carboxylic acid (B-43);

6-chloro-7-phenyl-2-trifluoromethyl-2H-1-benzopyran-3-carboxylic acid (B-44);

6,7-dichloro-2-trifluoromethyl-2H-1-benzopyran-3-carboxylic acid (B-45);

6,8-dichloro-2-trifluoromethyl-2H-1-benzopyran-3-carboxylic acid (B-46);

6-chloro-8-methyl-2-trifluoromethyl-2H-1-benzopyran-3-carboxylic acid (B-47);

8-chloro-6-methyl-2-trifluoromethyl-2H-1-benzopyran-3-carboxylic acid (B-48)

8-chloro-6-methoxy-2-trifluoromethyl-2H-1-benzopyran-3-carboxylic acid (B-49);

6-bromo-8-chloro-2-trifluoromethyl-2H-1-benzopyran-3-carboxylic acid (B-50);

8-bromo-6-fluoro-2-trifluoromethyl-2H-1-benzopyran-3-carboxylic acid (B-51);

8-bromo-6-methyl-2-trifluoromethyl-2H-1-benzopyran-3-carboxylic acid (B-52);

8-bromo-5-fluoro-2-trifluoromethyl-2H-1-benzopyran-3-carboxylic acid (B-53);

6-chloro-8-fluoro-2-trifluoromethyl-2H-1-benzopyran-3-carboxylic acid (B-54);

6-bromo-8-methoxy-2-trifluoromethyl-2H-1-benzopyran-3-carboxylic acid (B-55);

6-[[(phenylmethyl)amino]sulfonyl]-2-trifluoromethyl-2H-1-benzopyran-3-carboxylic acid (B-56);

6-[(dimethylamino)sulfonyl]-2-trifluoromethyl-2H-1-benzopyran-3-carboxylic acid (B-57);

6-[(methylamino)sulfonyl]-2-trifluoromethyl-2H-1-benzopyran-3-carboxylic acid (B-58);

6-[(4-morpholino)sulfonyl]-2-trifluoromethyl-2H-1-benzopyran-3-carboxylic acid (B-59);

6-[(1,1-dimethylethyl)aminosulfonyl]-2-trifluoromethyl-2H-1-benzopyran-3-carboxylic acid (B-60);

6-[(2-methylpropyl)aminosulfonyl]-2-trifluoromethyl-2H-1-benzopyran-3-carboxylic acid (B-61);

6-methylsulfonyl-2-trifluoromethyl-2H-1-benzopyran-3-carboxylic acid (B-62);

8-chloro-6-[[(phenylmethyl)amino]sulfonyl]-2-trifluoromethyl-2H-1-benzopyran-3-carboxylic acid (B-63);

6-phenylacetyl-2-trifluoromethyl-2H-1-benzopyran-3-carboxylic acid (B-64);

6,8-dibromo-2-trifluoromethyl-2H-1-benzopyran-3-carboxylic acid (B-65);

8-chloro-5,6-dimethyl-2-trifluoromethyl-2H-1-benzopyran-3-carboxylic acid (B-66);

6,8-dichloro-(S)-2-trifluoromethyl-2H-1-benzopyran-3-carboxylic acid (B-67);

6-benzylsulfonyl-2-trifluoromethyl-2H-1-benzopyran-3-carboxylic acid (B-68);

6-[[N-(2-furylmethyl)amino]sulfonyl]-2-trifluoromethyl-2H-1-benzopyran-3-carboxylic acid (B-69);

6-[[N-(2-phenylethyl)amino]sulfonyl]-2-trifluoromethyl-2H-1-benzopyran-3-carboxylic acid (B-70);

6-iodo-2-trifluoromethyl-2H-1-benzopyran-3-carboxylic acid (B-71);

7-(1,1-dimethylethyl)-2-pentafluoroethyl-2H-1-benzopyran-3-carboxylic acid (B-72);

6-chloro-2-trifluoromethyl-2H-1-benzothiopyran-3-carboxylic acid (B-73);

3-[(3-Chloro-phenyl)-(4-methanesulfonyl-phenyl)-methylene]-dihydro-furan-2-one or BMS-347070 (B-74);

8-acetyl-3-(4-fluorophenyl)-2-(4-methylsulfonyl)phenyl-imidazo(1,2-a)pyridine (B-75);

5,5-dimethyl-4-(4-methylsulfonyl)phenyl-3-phenyl-2-(5H)-furanone (B-76);

5-(4-fluorophenyl)-1-[4-(methylsulfonyl)phenyl]-3-(trifluoromethyl)pyrazole (B-77);

4-(4-fluorophenyl)-5-[4-(methylsulfonyl)phenyl]-1-phenyl-3-(trifluoromethyl)pyrazole (B-78);

4-(5-(4-chlorophenyl)-3-(4-methoxyphenyl)-1H-pyrazol-1-yl)benzenesulfonamide (B-79);

4-(3,5-bis(4-methylphenyl)-1H-pyrazol-1-yl)benzenesulfonamide (B-80);

4-(5-(4-chlorophenyl)-3-phenyl-1H-pyrazol-1-yl)benzenesulfonamide (B-81);

4-(3,5-bis(4-methoxyphenyl)-1H-pyrazol-1-yl)benzenesulfonamide (B-82);

4-(5-(4-chlorophenyl)-3-(4-methylphenyl)-1H-pyrazol-1-yl)benzenesulfonamide (B-83);

4-(5-(4-chlorophenyl)-3-(4-nitrophenyl)-1H-pyrazol-1-yl)benzenesulfonamide (B-84);

4-(5-(4-chlorophenyl)-3-(5-chloro-2-thienyl)-1H-pyrazol-1-yl)benzenesulfonamide (B-85);

4-(4-chloro-3,5-diphenyl-1H-pyrazol-1-yl)benzenesulfonamide (B-86);

4-[5-(4-chlorophenyl)-3-(trifluoromethyl)-1H-pyrazol-1-yl]benzenesulfonamide (B-87);

4-[5-phenyl-3-(trifluoromethyl)-1H-pyrazol-1-yl]benzenesulfonamide (B-88);

4-[5-(4-fluorophenyl)-3-(trifluoromethyl)-1H-pyrazol-1-yl]benzenesulfonamide (B-89);

4-[5-(4-methoxyphenyl)-3-(trifluoromethyl)-1H-pyrazol-1-yl]benzenesulfonamide (B-90);

4-[5-(4-chlorophenyl)-3-(difluoromethyl)-1H-pyrazol-1-yl]benzenesulfonamide (B-91);

4-[5-(4-methylphenyl)-3-(trifluoromethyl)-1H-pyrazol-1-yl]benzenesulfonamide (B-92);

4-[4-chloro-5-(4-chlorophenyl)-3-(trifluoromethyl)-1H-pyrazol-1-yl]benzenesulfonamide (B-93);

4-[3-(difluoromethyl)-5-(4-methylphenyl)-1H-pyrazol-1-yl]benzenesulfonamide (B-94);

4-[3-(difluoromethyl)-5-phenyl-1H-pyrazol-1-yl]benzenesulfonamide (B-95);

4-[3-(difluoromethyl)-5-(4-methoxyphenyl)-1H-pyrazol-1-yl]benzenesulfonamide (B-96);

4-[3-cyano-5-(4-fluorophenyl)-1H-pyrazol-1-yl]benzenesulfonamide (B-97);

4-[3-(difluoromethyl)-5-(3-fluoro-4-methoxyphenyl)-1H-pyrazol-1-yl]benzenesulfonamide (B-98);

4-[5-(3-fluoro-4-methoxyphenyl)-3-(trifluoromethyl)-1H-pyrazol-1-yl]benzenesulfonamide (B-99);

4-[4-chloro-5-phenyl-1H-pyrazol-1-yl]benzenesulfonamide (B-100);

4-[5-(4-chlorophenyl)-3-(hydroxymethyl)-1H-pyrazol-1-yl]benzenesulfonamide (B-101);

4-[5-(4-(N,N-dimethylamino)phenyl)-3-(trifluoromethyl)-1H-pyrazol-1-yl]benzenesulfonamide (B-102);

5-(4-fluorophenyl)-6-[4-(methylsulfonyl)phenyl]spiro[2.4]hept-5-ene (B-103);

4-[6-(4-fluorophenyl)spiro[2.4]hept-5-en-5-yl]benzenesulfonamide (B-104);

6-(4-fluorophenyl)-7-[4-(methylsulfonyl)phenyl]spiro[3.4]oct-6-ene (B-105);

5-(3-chloro-4-methoxyphenyl)-6-[4-(methylsulfonyl)phenyl]spiro[2.4]hept-5-ene (B-106);

4-[6-(3-chloro-4-methoxyphenyl)spiro[2.4]hept-5-en-5-yl]benzenesulfonamide (B-107);

5-(3,5-dichloro-4-methoxyphenyl)-6-[4-(methylsulfonyl)phenyl]spiro[2.4]hept-5-ene (B-108);

5-(3-chloro-4-fluorophenyl)-6-[4-(methylsulfonyl)phenyl]spiro[2.4]hept-5-ene (B-109);

4-[6-(3,4-dichlorophenyl)spiro[2.4]hept-5-en-5-yl]benzenesulfonamide (B-110);

2-(3-chloro-4-fluorophenyl)-4-(4-fluorophenyl)-5-(4-methylsulfonylphenyl)thiazole (B-111);

2-(2-chlorophenyl)-4-(4-fluorophenyl)-5-(4-methylsulfonylphenyl)thiazole (B-112);

5-(4-fluorophenyl)-4-(4-methylsulfonylphenyl)-2-methylthiazole (B-113);

4-(4-fluorophenyl)-5-(4-methylsulfonylphenyl)-2-trifluoromethylthiazole (B-114);

4-(4-fluorophenyl)-5-(4-methylsulfonylphenyl)-2-(2-thienyl)thiazole (B-115);

4-(4-fluorophenyl)-5-(4-methylsulfonylphenyl)-2-benzylaminothiazole (B-116);

4-(4-fluorophenyl)-5-(4-methylsulfonylphenyl)-2-(1-propylamino)thiazole (B-117);

2-[(3,5-dichlorophenoxy)methyl)-4-(4-fluorophenyl)-5-[4-(methylsulfonyl)phenyl]thiazole (B-118);

5-(4-fluorophenyl)-4-(4-methylsulfonylphenyl)-2-trifluoromethylthiazole (B-119);

1-methylsulfonyl-4-[1,1-dimethyl-4-(4-fluorophenyl)cyclopenta-2,4-dien-3-yl]benzene (B-120);

4-[4-(4-fluorophenyl)-1,1-dimethylcyclopenta-2,4-dien-3-yl]benzenesulfonamide (B-121);

5-(4-fluorophenyl)-6-[4-(methylsulfonyl)phenyl]spiro[2.4]hepta-4,6-diene (B-122);

4-[6-(4-fluorophenyl)spiro[2.4]hepta-4,6-dien-5-yl]benzenesulfonamide (B-123);

6-(4-fluorophenyl)-2-methoxy-5-[4-(methylsulfonyl)phenyl]-pyridine-3-carbonitrile (B-124);

2-bromo-6-(4-fluorophenyl)-5-[4-(methylsulfonyl)phenyl]-pyridine-3-carbonitrile (B-125);

6-(4-fluorophenyl)-5-[4-(methylsulfonyl)phenyl]-2-phenyl-pyridine-3-carbonitrile (B-126);

4-[2-(4-methylpyridin-2-yl)-4-(trifluoromethyl)-1H-imidazol-1-yl]benzenesulfonamide (B-127);

4-[2-(5-methylpyridin-3-yl)-4-(trifluoromethyl)-1H-imidazol-1-yl]benzenesulfonamide (B-128);

4-[2-(2-methylpyridin-3-yl)-4-(trifluoromethyl)-1H-imidazol-1-yl]benzenesulfonamide (B-129);

3-[1-[4-(methylsulfonyl)phenyl]-4-(trifluoromethyl)-1H-imidazol-2-yl]pyridine (B-130);

2-[1-[4-(methylsulfonyl)phenyl-4-(trifluoromethyl)-1H-imidazol-2-yl]pyridine (B-131);

2-methyl-4-[1-[4-(methylsulfonyl)phenyl-4-(trifluoromethyl)-1H-imidazol-2-yl]pyridine (B-132);

2-methyl-6-[1-[4-(methylsulfonyl)phenyl-4-(trifluoromethyl)-1H-imidazol-2-yl]pyridine (B-133);

4-[2-(6-methylpyridin-3-yl)-4-(trifluoromethyl)-1H-imidazol-1-yl]benzenesulfonamide (B-134);

2-(3,4-difluorophenyl)-1-[4-(methylsulfonyl)phenyl]-4-(trifluoromethyl)-1H-imidazole (B-135);

4-[2-(4-methylphenyl)-4-(trifluoromethyl)-1H-imidazol-1-yl]benzenesulfonamide (B-136);

2-(4-chlorophenyl)-1-[4-(methylsulfonyl)phenyl]-4-methyl-1H-imidazole (B-137);

2-(4-chlorophenyl)-1-[4-(methylsulfonyl)phenyl]-4-phenyl-1H-imidazole (B-138);

2-(4-chlorophenyl)-4-(4-fluorophenyl)-1-[4-(methylsulfonyl)phenyl]-1H-imidazole (B-139);

2-(3-fluoro-4-methoxyphenyl)-1-[4-(methylsulfonyl)phenyl-4-(trifluoromethyl)-1H-imidazole (B-140);

1-[4-(methylsulfonyl)phenyl]-2-phenyl-4-trifluoromethyl-1H-imidazole (B-141);

2-(4-methylphenyl)-1-[4-(methylsulfonyl)phenyl]-4-trifluoromethyl-1H-imidazole (B-142);

4-[2-(3-chloro-4-methylphenyl)-4-(trifluoromethyl)-1H-imidazol-1-yl]benzenesulfonamide (B-143);

2-(3-fluoro-5-methylphenyl)-1-[4-(methylsulfonyl)phenyl]-4-(trifluoromethyl)-1H-imidazole (B-144);

4-[2-(3-fluoro-5-methylphenyl)-4-(trifluoromethyl)-1H-imidazol-1-yl]benzenesulfonamide (B-145);

2-(3-methylphenyl)-1-[4-(methylsulfonyl)phenyl]-4-trifluoromethyl-1H-imidazole (B-146);

4-[2-(3-methylphenyl)-4-trifluoromethyl-1H-imidazol-1-yl]benzenesulfonamide (B-147);

1-[4-(methylsulfonyl)phenyl]-2-(3-chlorophenyl)-4-trifluoromethyl-1H-imidazole (B-148);

4-[2-(3-chlorophenyl)-4-trifluoromethyl-1H-imidazol-1-yl]benzenesulfonamide (B-149);

4-[2-phenyl-4-trifluoromethyl-1H-imidazol-1-yl]benzenesulfonamide (B-150);

4-[2-(4-methoxy-3-chlorophenyl)-4-trifluoromethyl-1H-imidazol-1-yl]benzenesulfonamide (B-151);

1-allyl-4-(4-fluorophenyl)-3-[4-(methylsulfonyl)phenyl]-5-(trifluoromethyl)-1H-pyrazole (B-152);

4-[1-ethyl-4-(4-fluorophenyl)-5-(trifluoromethyl)-1H-pyrazol-3-yl]benzenesulfonamide (B-153);

N-phenyl-[4-(4-fluorophenyl)-3-[4-(methylsulfonyl)phenyl]-5-(trifluoromethyl)-1H-pyrazol-1-yl]acetamide (B-154);

ethyl [4-(4-fluorophenyl)-3-[4-(methylsulfonyl)phenyl]-5-(trifluoromethyl)-1H-pyrazol-1-yl]acetate (B-155);

4-(4-fluorophenyl)-3-[4-(methylsulfonyl)phenyl]-1-(2-phenylethyl)-1H-pyrazole (B-156);

4-(4-fluorophenyl)-3-[4-(methylsulfonyl)phenyl]-1-(2-phenylethyl)-5-(trifluoromethyl)pyrazole (B-157);

1-ethyl-4-(4-fluorophenyl)-3-[4-(methylsulfonyl)phenyl]-5-(trifluoromethyl)-1H-pyrazole (B-158);

5-(4-fluorophenyl)-4-(4-methylsulfonylphenyl)-2-trifluoromethyl-1H-imidazole (B-159);

4-[4-(methylsulfonyl)phenyl]-5-(2-thiophenyl)-2-(trifluoromethyl)-1H-imidazole (B-160);

5-(4-fluorophenyl)-2-methoxy-4-[4-(methylsulfonyl)phenyl]-6-(trifluoromethyl)pyridine (B-161);

2-ethoxy-5-(4-fluorophenyl)-4-[4-(methylsulfonyl)phenyl]-6-(trifluoromethyl)pyridine (B-162);

5-(4-fluorophenyl)-4-[4-(methylsulfonyl)phenyl]-2-(2-propynyloxy)-6-(trifluoromethyl)pyridine (B-163);

2-bromo-5-(4-fluorophenyl)-4-[4-(methylsulfonyl)phenyl]-6-(trifluoromethyl)pyridine (B-164);

4-[2-(3-chloro-4-methoxyphenyl)-4,5-difluorophenyl]benzenesulfonamide (B-165);

1-(4-fluorophenyl)-2-[4-(methylsulfonyl)phenyl]benzene (B-166);

5-difluoromethyl-4-(4-methylsulfonylphenyl)-3-phenylisoxazole (B-167);

4-[3-ethyl-5-phenylisoxazol-4-yl]benzenesulfonamide (B-168);

4-[5-difluoromethyl-3-phenylisoxazol-4-yl]benzenesulfonamide (B-169);

4-[5-hydroxymethyl-3-phenylisoxazol-4-yl]benzenesulfonamide (B-170);

4-[5-methyl-3-phenyl-isoxazol-4-yl]benzenesulfonamide (B-171);

1-[2-(4-fluorophenyl)cyclopenten-1-yl]-4-(methylsulfonyl)benzene (B-172);

1-[2-(4-fluoro-2-methylphenyl)cyclopenten-1-yl]-4-(methylsulfonyl)benzene (B-173);

1-[2-(4-chlorophenyl)cyclopenten-1-yl]-4-(methylsulfonyl)benzene (B-174);

1-[2-(2,4-dichlorophenyl)cyclopenten-1-yl]-4-(methylsulfonyl)benzene (B-175);

1-[2-(4-trifluoromethylphenyl)cyclopenten-1-yl]-4-(methylsulfonyl)benzene (B-176);

1-[2-(4-methylthiophenyl)cyclopenten-1-yl]-4-(methylsulfonyl)benzene (B-177);

1-[2-(4-fluorophenyl)-4,4-dimethylcyclopenten-1-yl]-4-(methylsulfonyl)benzene (B-178);

4-[2-(4-fluorophenyl)-4,4-dimethylcyclopenten-1-yl]benzenesulfonamide (B-179);

1-[2-(4-chlorophenyl)-4,4-dimethylcyclopenten-1-yl]-4-(methylsulfonyl)benzene (B-180);

4-[2-(4-chlorophenyl)-4,4-dimethylcyclopenten-1-yl]benzenesulfonamide (B-181);

4-[2-(4-fluorophenyl)cyclopenten-1-yl]benzenesulfonamide (B-182);

4-[2-(4-chlorophenyl)cyclopenten-1-yl]benzenesulfonamide (B-183);

1-[2-(4-methoxyphenyl)cyclopenten-1-yl]-4-(methylsulfonyl)benzene (B-184);

1-[2-(2,3-difluorophenyl)cyclopenten-1-yl]-4-(methylsulfonyl)benzene (B-185);

4-[2-(3-fluoro-4-methoxyphenyl)cyclopenten-1-yl]benzenesulfonamide (B-186);

1-[2-(3-chloro-4-methoxyphenyl)cyclopenten-1-yl]-4-(methylsulfonyl)benzene (B-187);

4-[2-(3-chloro-4-fluorophenyl)cyclopenten-1-yl]benzenesulfonamide (B-188);

4-[2-(2-methylpyridin-5-yl)cyclopenten-1-yl]benzenesulfonamide (B-189);

ethyl 2-[4-(4-fluorophenyl)-5-[4-(methylsulfonyl) phenyl]oxazol-2-yl]-2-benzyl-acetate (B-190);

2-[4-(4-fluorophenyl)-5-[4-(methylsulfonyl)phenyl]oxazol-2-yl]acetic acid (B-191);

2-(tert-butyl)-4-(4-fluorophenyl)-5-[4-(methylsulfonyl)phenyl]oxazole (B-192);

4-(4-fluorophenyl)-5-[4-(methylsulfonyl)phenyl]-2-phenyloxazole (B-193);

4-(4-fluorophenyl)-2-methyl-5-[4-(methylsulfonyl)phenyl]oxazole (B-194);

4-[5-(3-fluoro-4-methoxyphenyl)-2-trifluoromethyl-4-oxazolyl]benzenesulfonamide (B-195);

6-chloro-7-(1,1-dimethylethyl)-2-trifluoromethyl-2H-1-benzopyran-3-carboxylic acid (B-196);

6-chloro-8-methyl-2-trifluoromethyl-2H-1-benzopyran-3-carboxylic acid (B-197);

5,5-dimethyl-3-(3-fluorophenyl)-4-methylsulfonyl-2(5H)-furanone (B-198);

6-chloro-2-trifluoromethyl-2H-1-benzothiopyran-3-carboxylic acid (B-199);

4-[5-(4-chlorophenyl)-3-(trifluoromethyl)-1H-pyrazol-1-yl]benzenesulfonamide (B-200);

4-[5-(4-methylphenyl)-3-(trifluoromethyl)-1H-pyrazol-1-yl]benzenesulfonamide (B-201);

4-[5-(3-fluoro-4-methoxyphenyl)-3-(difluoromethyl)-1H-pyrazol-1-yl]benzenesulfonamide (B-202);

3-[1-[4-(methylsulfonyl)phenyl]-4-trifluoromethyl-1H-imidazol-2-yl]pyridine (B-203);

2-methyl-5-[1-[4-(methylsulfonyl)phenyl]-4-trifluoromethyl-1H-imidazol-2-yl]pyridine (B-204);

4-[2-(5-methylpyridin-3-yl)-4-(trifluoromethyl)-1H-imidazol-1-yl]benzenesulfonamide (B-205);

4-[5-methyl-3-phenylisoxazol-4-yl]benzenesulfonamide (B-206);

4-[5-hydroxymethyl-3-phenylisoxazol-4-yl]benzenesulfonamide (B-207);

[2-trifluoromethyl-5-(3,4-difluorophenyl)-4-oxazolyl]benzenesulfonamide (B-208);

4-[2-methyl-4-phenyl-5-oxazolyl]benzenesulfonamide (B-209);

4-[5-(2-fluoro-4-methoxyphenyl)-2-trifluoromethyl-4-oxazolyl]benzenesulfonamide (B-210);

[2-(2-chloro-6-fluoro-phenylamino)-5-methyl-phenyl]-acetic acid or COX 189 (B-211);

N-(4-Nitro-2-phenoxy-phenyl)-methanesulfonamide or nimesulide (B-212);

N-[6-(2,4-difluoro-phenoxy)-1-oxo-indan-5-yl]-methanesulfonamide or flosulide (B-213);

N-[6-(2,4-Difluoro-phenylsulfanyl)-1-oxo-1H-inden-5-yl]-methanesulfonamide, soldium salt or L-745337 (B-214);

N-[5-(4-fluoro-phenylsulfanyl)-thiophen-2-yl]-methanesulfonamide or RWJ-63556 (B-215);

3-(3,4-Difluoro-phenoxy)-4-(4-methanesulfonyl-phenyl)-5-methyl-5-(2,2,2-trifluoroethyl)-5H-furan-2-one or L-784512 or L-784512 (B-216);

(5Z)-2-amino-5-[[3,5-bis(1,1-dimethylethyl)-4-hydroxyphenyl]methylene]-4(5H)-thiazolone or darbufelone (B-217);

CS-502 (B-218);

LAS-34475 (B-219);

LAS-34555 (B-220);

S-33516 (B-221);

SD-8381 (B-222);

L-783003 (B-223);

N-[3-(formylamino)-4-oxo-6-phenoxy-4H-1-benzopyran-7-yl]-methanesulfonamide or

T-614 (B-224);

D-1367 (B-225);

L-748731 (B-226);

(6aR,10aR)-3-(1,1-dimethylheptyl)-6a,7,10,10a-tetrahydro-1-hydroxy-6,6-dimethyl-6H-dibenzo[b,d]pyran-9-carboxylic acid or CT3 (B-227);

CGP-28238 (B-228);

4-[[3,5-bis(1,1-dimethylethyl)-4-hydroxyphenyl]methylene]dihydro-2-methyl-2H-1,2-oxazin-3(4H)-one or BF-389 (B-229);

GR-253035 (B-230);

6-dioxo-9H-purin-8-yl-cinnamic acid (B-231);

S-2474 (B-232);

4-[4-(methyl)-sulfonyl)phenyl]-3-phenyl-2(5H)-furanone;

4-(5-methyl-3-phenyl-4-isoxazolyl);

2-(6-methylpyrid-3-yl)-3-(4-methylsulfonylphenyl)-5-chloropyridine;

4-[5-(4-methylphenyl)-3-(trifluoromethyl)-1H-pyrazol-1-yl];

N-[[4-(5-methyl-3-phenyl-4-isoxazolyl)phenyl]sulfonyl];

4-[5-(3-fluoro-4-methoxyphenyl)-3-difluoromethyl)-1H-pyrazol-1-yl]benzenesulfonamide;

(S)-6,8-dichloro-2-(trifluoromethyl)-2H-1-benzopyran-3-carboxylic acid;

2-(3,4-difluorophenyl)-4-(3-hydroxy-3-methylbutoxy)-5-[4-(methylsulfonyl)phenyl]-3(2H)-pyridzainone;

2-trifluoromethyl-3H-naptho[2,1-b]pyran-3-carboxylic acid;

6-chloro-7-(1,1-dimethylethyl)-2-trifluoromethyl-2H-1-benzopyran-3-carboxylic acid;

[2-(2,4-dichloro-6-ethyl-3,5-dimethyl-phenylamino)-5-propyl-phenyl]-acetic acid;

or an isomer, a pharmaceutically acceptable salt, ester or prodrug thereof.

TABLE 3
Examples of Cyclooxygenase-2 Selective Inhibitors as Embodiments

| Compound | |
|---|---|
| Number | Structural Formula |
| B-26 | 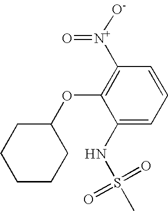 |
| | N-(2-cyclohexyloxynitrophenyl) methane sulfonamide or |
| | NS-398; |
| B-27 | 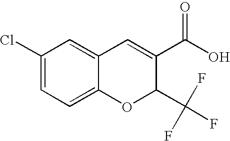 |
| | 6-chloro-2-trifluoromethyl-2H-1-benzopyran-3- |
| | carboxylic acid; |
| B-28 | 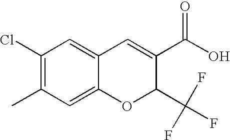 |
| | 6-chloro-7-methyl-2-trifluoromethyl-2H-1-benzopyran- |
| | 3-carboxylic acid; |
| B-29 | 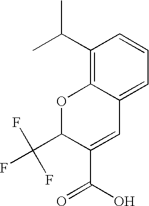 |
| | 8-(1-methylethyl)-2-trifluoromethyl-2H-1-benzopyran- |
| | 3-carboxylic acid; |
| B-30 | 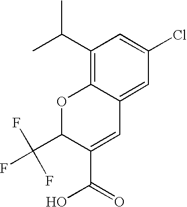 |
| | 6-chloro-8-(1-methylethyl)-2-trifluoromethyl- |
| | 2H-1-benzopyran-3-carboxylic acid; |
| B-31 | 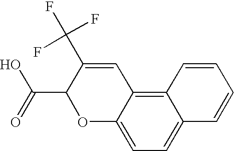 |
| | 2-trifluoromethyl-3H-naphtho[2,1-b]pyran-3- |
| | carboxylic acid; |
| B-32 | 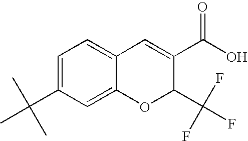 |
| | 7-(1,1-dimethylethyl)-2-trifluoromethyl-2H-1- |
| | benzopyran-3-carboxylic acid; |
| B-33 | 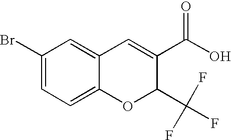 |
| | 6-bromo-2-trifluoromethyl-2H-1-benzopyran-3- |
| | carboxylic acid; |
| B-34 | 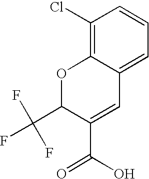 |
| | 8-chloro-2-trifluoromethyl-2H-1-benzopyran-3- |
| | carboxylic acid; |
| B-35 | 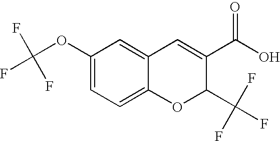 |
| | 6-trifluoromethoxy-2-trifluoromethyl-2H-1- |
| | benzopyran-3-carboxylic acid; |
| B-36 | 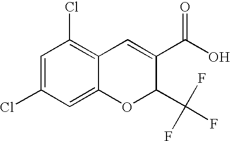 |
| | 5,7-dichloro-2-trifluoromethyl-2H-1-benzopyran-3- |
| | carboxylic acid; |
| B-37 | 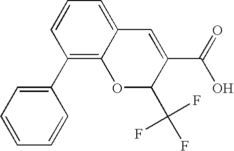 |
| | 8-phenyl-2-trifluoromethyl-2H-1-benzopyran-3- |
| | carboxylic acid; |
| B-38 | 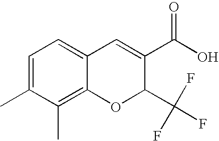 |
| | 7,8-dimethyl-2-trifluoromethyl-2H-1-benzopyran- |
| | 3-carboxylic acid; |
| B-39 | 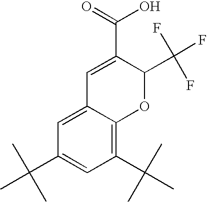 |
| | 6,8-bis(dimethylethyl)-2-trifluoromethyl-2H-1- |
| | benzopyran-3-carboxylic acid; |
| B-40 | 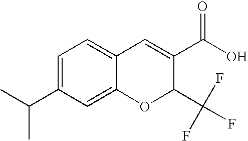 |
| | 7-(1-methylethyl)-2-trifluoromethyl-2H-1-benzopyran- |
| | 3-carboxylic acid; |
| B-41 | 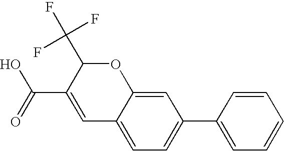 |
| | 7-phenyl-2-trifluoromethyl-2H-1-benzopyran-3- |
| | carboxylic acid; |
| B-42 | 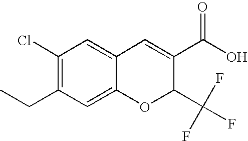 |
| | 6-chloro-7-ethyl-2-trifluoromethyl-2H-1-benzopyran- |
| | 3-carboxylic acid; |
| B-43 | 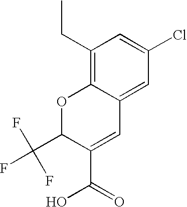 |
| | 6-chloro-8-ethyl-2-trifluoromethyl-2H-1-benzopyran- |
| | 3-carboxylic acid; |
| B-44 | 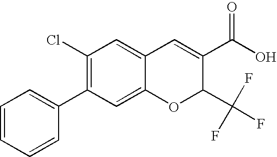 |
| | 6-chloro-7-phenyl-2-trifluoromethyl-2H-1-benzopyran- |
| | 3-carboxylic acid; |
| B-45 | 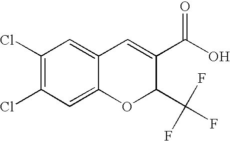 |
| | 6,7-dichloro-2-trifluoromethyl-2H-1-benzopyran-3- |
| | carboxylic acid; |
| B-46 | 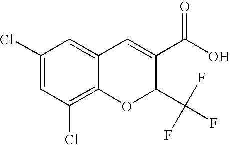 |
| | 6,8-dichloro-2-trifluoromethyl-2H-1-benzopyran-3- |
| | carboxylic acid; |
| B-47 | 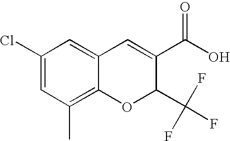 |
| | 6-chloro-8-methyl-2-trifluoromethyl-2H-1-benzopyran- |
| | 3-carboxylic acid; |
| B-48 | 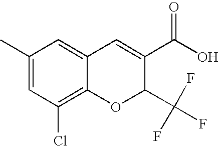 |
| | 8-chloro-6-methyl-2-trifluoromethyl-2H-1-benzopyran- |
| | 3-carboxylic acid; |
| B-49 | 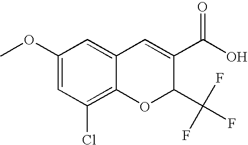 |
| | 8-chloro-6-methoxy-2-trifluoromethyl-2H-1-benzopyran- |
| | 3-carboxylic acid; |
| B-50 | 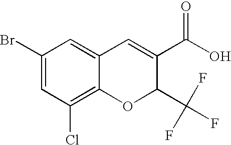 |
| | 6-bromo-8-chloro-2-trifluoromethyl-2H-1-benzopyran-3- |
| | carboxylic acid; |
| B-51 | 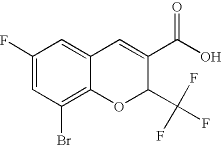 |
| | 8-bromo-6-fluoro-2-trifluoromethyl-2H-1-benzopyran-3- |
| | carboxylic acid; |
| B-52 | 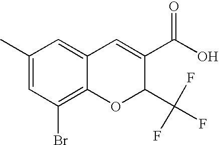 |
| | 8-bromo-6-methyl-2-trifluoromethyl-2H-1-benzopyran-3- |
| | carboxylic acid; |
| B-53 | 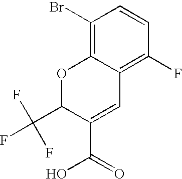 |
| | 8-bromo-5-fluoro-2-trifluoromethyl-2H-1-benzopyran-3- |
| | carboxylic acid; |
| B-54 | 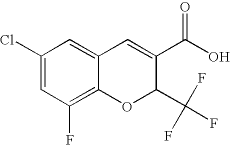 |
| | 6-chloro-8-fluoro-2-trifluoromethyl-2H-1-benzopyran- |
| | 3-carboxylic acid; |
| B-55 | 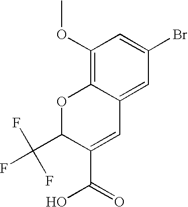 |
| | 6-bromo-8-methoxy-2-trifluoromethyl-2H-1-benzopyran-3- |
| | carboxylic acid; |
| B-56 | 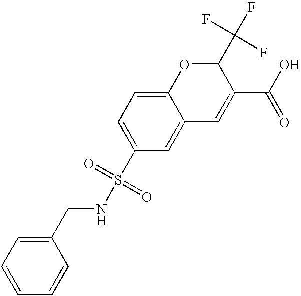 |
| | 6-[[(phenylmethyl)amino]sulfonyl]-2-trifluoromethyl- |
| | 2H-1-benzopyran-3-carboxylic acid; |
| B-57 | 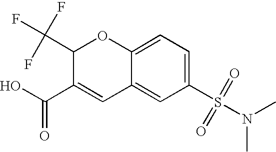 |
| | 6-[(dimethylamino)sulfonyl]-2-trifluoromethyl-2H-1- |
| | benzopyran-3-carboxylic acid; |
| B-58 | 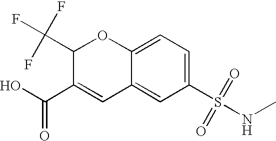 |
| | 6-[(methylamino)sulfonyl]-2-trifluoromethyl-2H-1- |
| | benzopyran-3-carboxylic acid; |
| B-59 | 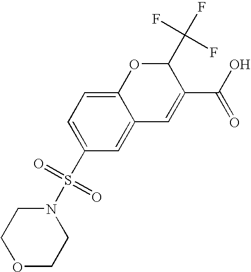 |
| | 6-[(4-morpholino)sulfonyl]-2-trifluoromethyl-2H-1- |
| | benzopyran-3-carboxylic acid; |
| B-60 | 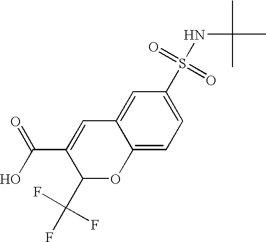 |
| | 6-[(1,1-dimethylethyl)aminosulfonyl]-2-trifluoromethyl- |
| | 2H-1-benzopyran-3-carboxylic acid; |
| B-61 | 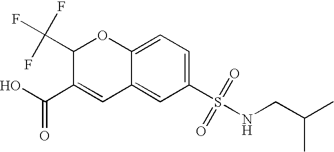 |
| | 6-[(2-methylpropyl)aminosulfonyl]-2-trifluoromethyl- |
| | 2H-1-benzopyran-3-carboxylic acid; |
| B-62 | 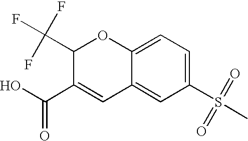 |
| | 6-methylsulfonyl-2-trifluoromethyl-2H-1-benzopyran-3- |
| | carboxylic acid; |
| B-63 | 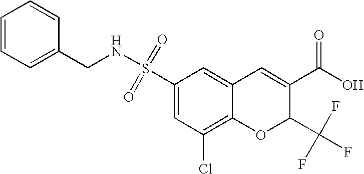 |
| | 8-chloro-6-[[(phenylmethyl)amino]sulfonyl]-2- |
| | trifluoromethyl-2H-1-benzopyran-3-carboxylic acid; |
| B-64 | 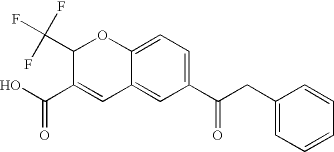 |
| | 6-phenylacetyl-2-trifluoromethyl-2H-1-benzopyran-3- |
| | carboxylic acid; |
| B-65 | 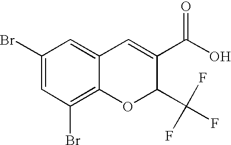 |
| | 6,8-dibromo-2-trifluoromethyl-2H-1-benzopyran-3- |
| | carboxylic acid; |
| B-66 | 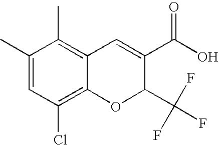 |
| | 8-chloro-5,6-dimethyl-2-trifluoromethyl-2H-1- |
| | benzopyran-3-carboxylic acid, |
| B-67 | 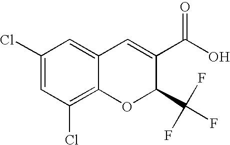 |
| | 6,8-dichloro-(S)-2-trifluoromethyl-2H-1-benzopyran- |
| | 3-carboxylic acid; |
| B-68 | 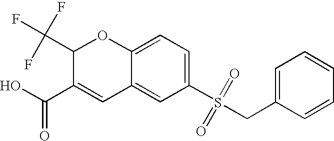 |
| | 6-benzylsulfonyl-2-trifluoromethyl-2H-1-benzopyran- |
| | 3-carboxylic acid; |
| B-69 | 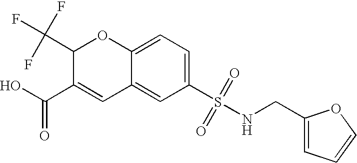 |
| | 6-[[N-(2-furylmethyl)amino]sulfonyl]-2-trifluoromethyl- |
| | 2H-1-benzopyran-3-carboxylic acid; |
| B-70 | 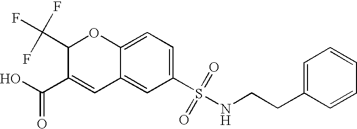 |
| | 6-[[N-(2-phenylethyl)amino]sulfonyl]-2-trifluoromethyl- |
| | 2H-1-benzopyran-3-carboxylic acid; |
| B-71 | 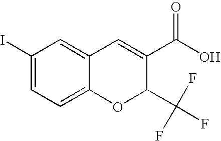 |
| | 6-iodo-2-trifluoromethyl-2H-1-benzopyran-3- |
| | carboxylic acid; |
| B-72 | 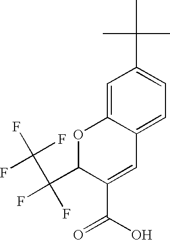 |
| | 7-(1,1-dimethylethyl)-2-pentafluoroethyl-2H-1-benzopyran- |
| | 3-carboxylic acid; |
| B-73 | 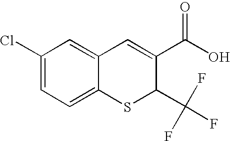 |
| | 6-chloro-2-trifluoromethyl-2H-1-benzothiopyran-3- |
| | carboxylic acid; |
| B-74 | 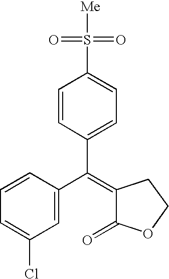 |
| | 3-[(3-chloro-phenyl)-(4-methanesulfonyl-phenyl)- |
| | methylene]-dihydro-furan-2-one or BMS-347070; |
| B-75 | 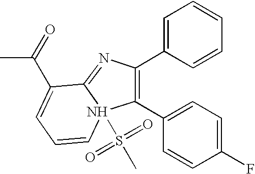 |
| | 8-acetyl-3-(4-fluorophenyl)-2-(4-methylsulfonyl)phenyl- |
| | imidazo(1,2-a)pyridine; |
| B-76 | 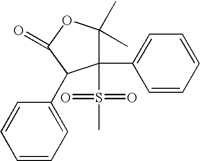 |
| | 5,5-dimethyl-4-(4-methylsulfonyl)phenyl-3-phenyl-2- |
| | (5H)-furanone; |
| B-77 | 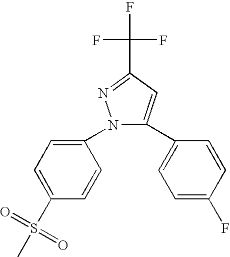 |
| | 5-(4-fluorophenyl)-1-[4-(methylsulfonyl)phenyl]-3- |
| | (trifluoromethyl)pyrazole; |
| B-78 | 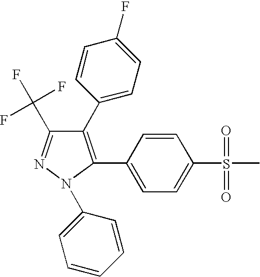 |
| | 4-(4-fluorophenyl)-5-[4-(methylsulfonyl)phenyl]- |
| | 1-phenyl-3-(trifluoromethyl)pyrazole; |
| B-79 | 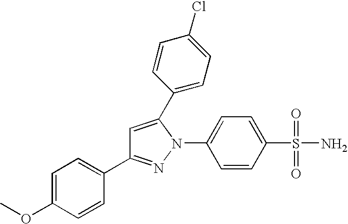 |
| | 4-(5-(4-chlorophenyl)-3-(4-methoxyphenyl)-1H-pyrazol- |
| | 1-yl)benzenesulfonamide; |
| B-80 | 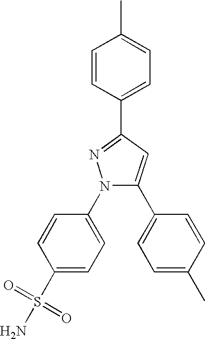 |
| | 4-(3,5-bis(4-methylphenyl)-1H-pyrazol-1-yl) |
| | benzenesulfonamide; |
| B-81 | 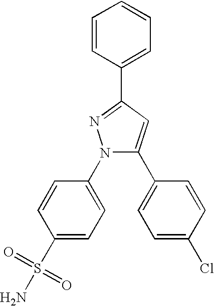 |
| | 4-(5-(4-chlorophenyl)-3-phenyl-1H-pyrazol-1-yl) |
| | benzenesulfonamide; |
| B-82 | 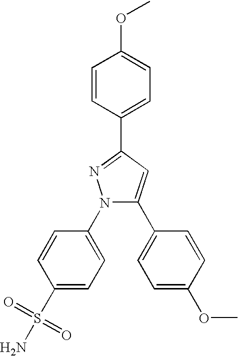 |
| | 4-(3,5-bis(4-methoxyphenyl)-1H-pyrazol-1-yl) |
| | benzenesulfonamide; |
| B-83 | 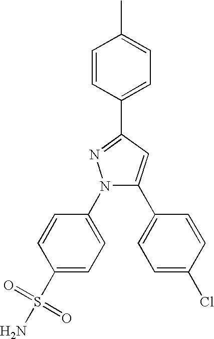 |
| | 4-(5-(4-chlorophenyl)-3-(4-methylphenyl)-1H-pyrazol- |
| | 1-yl)benzenesulfonamide; |
| B-84 | 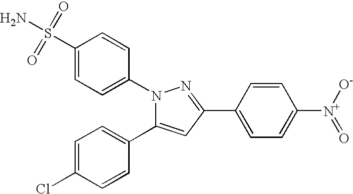 |
| | 4-(5-(4-chlorophenyl)-3-(4-nitrophenyl)-1H-pyrazol-1- |
| | yl)benzenesulfonamide; |
| B-85 | 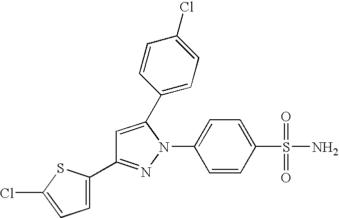 |
| | 4-(5-(4-chlorophenyl)-3-(5-chloro-2-thienyl)-1H-pyrazol- |
| | 1-yl)benzenesulfonamide; |
| B-86 | 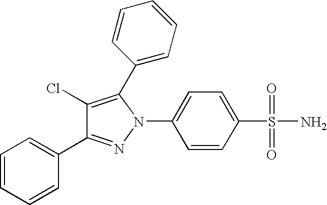 |
| | 4-(4-chloro-3,5-diphenyl-1H-pyrazol-1-yl) |
| | benzenesulfonamide; |
| B-87 | 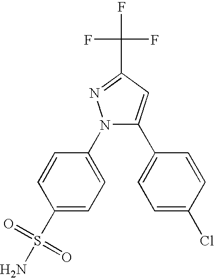 |
| | 4-[5-(4-chlorophenyl)-3-(trifluoromethyl)-1H-pyrazol-1- |
| | yl]benzenesulfonamide; |
| B-88 | 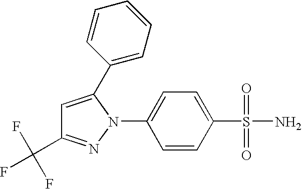 |
| | 4-[5-phenyl-3-(trifluoromethyl)-1H-pyrazol-1-yl] |
| | benzenesulfonamide; |
| B-89 | 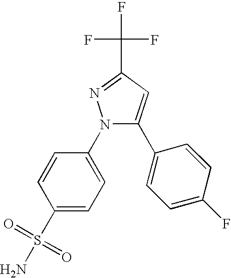 |
| | 4-[5-(4-fluorophenyl)-3-(trifluoromethyl)-1H- |
| | pyrazol-1-yl]benzenesulfonamide; |
| B-90 | 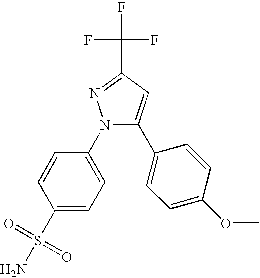 |
| | 4[5-(4-methoxyphenyl)-3-(trifluoromethyl)-1H-pyrazol- |
| | 1-yl]benzenesulfonamide; |
| B-91 | 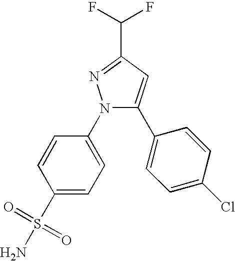 |
| | 4-[5-(4-chlorophenyl)-3-(difluoromethyl)-1H-pyrazol-1- |
| | yl]benzenesulfonamide; |
| B-92 | 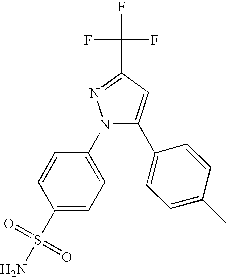 |
| | 4-[5-(4-methylphenyl)-3-(trifluoromethyl)-1H-pyrazol- |
| | 1-yl]benzenesulfonamide; |
| B-93 | 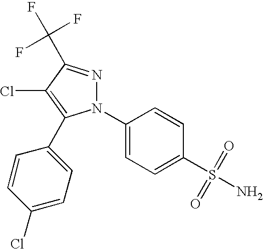 |
| | 4-[4-chloro-5-(4-chlorophenyl)-3-(trifluoromethyl)-1H- |
| | pyrazol-1-yl]benzenesulfonamide; |
| B-94 | 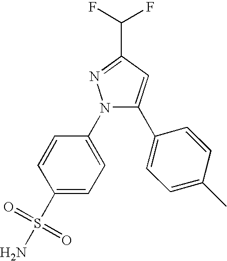 |
| | 4-[3-(difluoromethyl)-5-(4-methylphenyl)-1H-pyrazol- |
| | 1-yl]benzenesulfonamide; |
| B-95 | 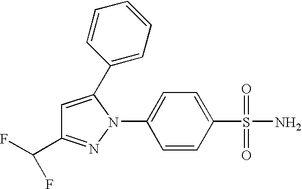 |
| | 4-[3-(difluoromethyl)-5-phenyl-1H-pyrazol-1-yl] |
| | benzenesulfonamide; |
| B-96 | 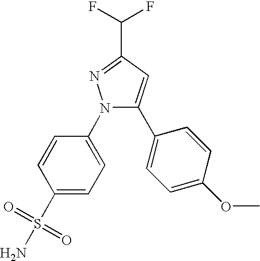 |
| | 4-[3-(difluoromethyl)-5-(4-methoxyphenyl)-1H- |
| | pyrazol-1-yl]benzenesulfonamide; |
| B-97 | 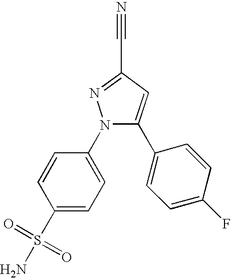 |
| | 4-[3-cyano-5-(4-fluorophenyl)-1H-pyrazol-1-yl] |
| | benzenesulfonamide; |
| B-98 | 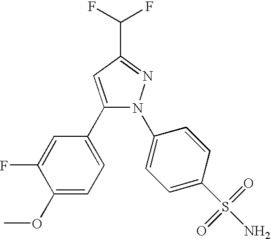 |
| | 4-[3-(difluoromethyl)-5-(3-fluoro-4-methoxyphenyl)- |
| | 1H-pyrazol-1-yl]benzenesulfonamide; |
| B-99 | 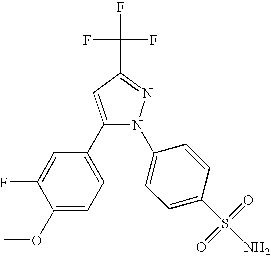 |
| | 4-[5-(3-fluoro-4-methoxyphenyl)-3-(trifluoromethyl)- |
| | 1H-pyrazol-1-yl]benzenesulfonamide; |
| B-100 | 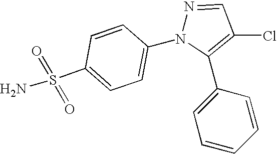 |
| | 4-[4-chloro-5-phenyl-1H-pyrazol-1-yl]benzenesulfonamide; |
| B-101 | 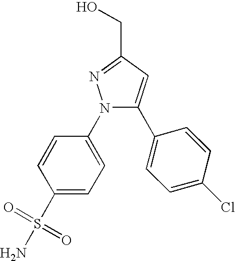 |
| | 4-[5-(4-chlorophenyl)-3-(hydroxymethyl)-1H-pyrazol-1- |
| | yl]benzenesulfonamide; |
| B-102 | 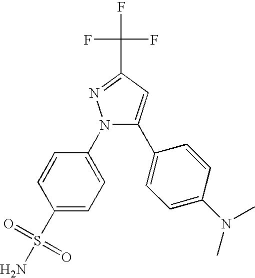 |
| | 4-[5-(4-(N,N-dimethylamino)phenyl)-3-(trifluoromethyl)- |
| | 1H-pyrazol-1-yl]benzenesulfonamide; |
| B-103 | 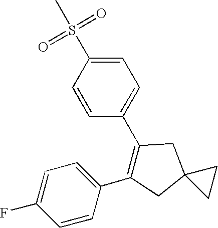 |
| | 5-(4-fluorophenyl)-6-[4-(methylsulfonyl)phenyl] |
| | spiro[2.4]hept-5-ene; |
| B-104 | 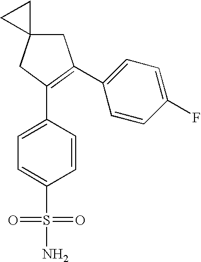 |
| | 4-[6-(4-fluorophenyl)spiro[2.4]hept-5-en-5- |
| | yl]benzenesulfonamide; |
| B-105 | 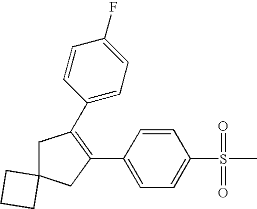 |
| | 6-(4-fluorophenyl)-7-[4-methylsulfonyl)phenyl] |
| | spiro[3.4]oct-6-ene; |
| B-106 | 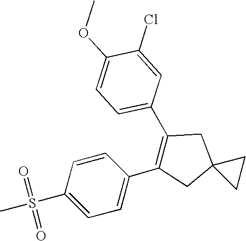 |
| | 5-(3-chloro-4-methoxyphenyl)-6-[4-(methylsulfonyl) |
| | phenyl]spiro[2.4]hept-5-ene; |
| B-107 | 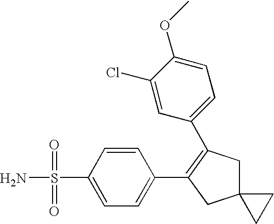 |
| | 4-[6-(3-chloro-4-methoxyphenyl)spiro[2.4]hept-5-en- |
| | 5-yl]benzenesulfonamide; |
| B-108 | 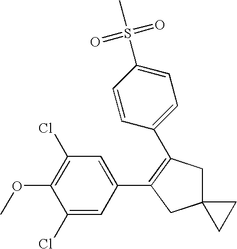 |
| | 5-(3,5-dichloro-4-methoxyphenyl)-6-[4-(methylsulfonyl) |
| | phenyl]spiro[2.4]hept-5-ene; |
| B-109 | 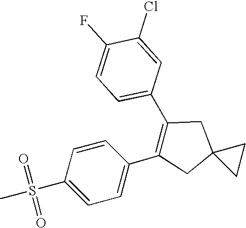 |
| | 5-(3-chloro-4-fluorophenyl)-6-[4-(methylsulfonyl)phenyl] |
| | spiro[2.4]hept-5-ene; |
| B-110 | 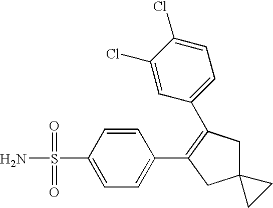 |
| | 4-[6-(3,4-dichlorophenyl)spiro[2.4]hept-5-en-5- |
| | yl]benzenesulfonamide; |
| B-111 | 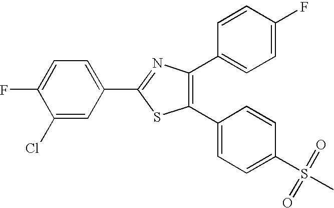 |
| | 2-(3-chloro-4-fluorophenyl)-4-(4-fluorophenyl)-5-(4- |
| | methylsulfonylphenyl)thiazole; |
| B-112 | 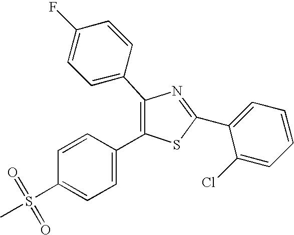 |
| | 2-(2-chloropbenyl)-4-(4-fluorophenyl)-5-(4- |
| | methylsulfonylphenyl)thiazole; |
| B-113 | 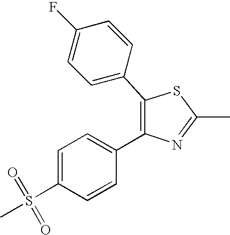 |
| | 5-(4-fluorophenyl)-4-(4-methylsulfonylphenyl)-2- |
| | methylthiazole; |
| B-114 | 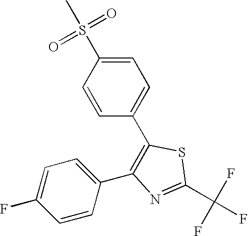 |
| | 4-(4-fluorophenyl)-5-(4-methylsulfonylphenyl)-2- |
| | trifluoromethylthiazole; |
| B-115 | 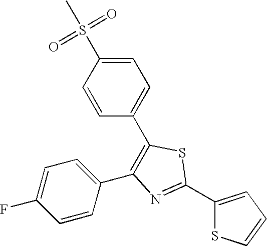 |
| | 4-(4-fluorophenyl)-5-(4-methylsulfonylphenyl)-2- |
| | (2-thienyl)thiazole; |
| B-116 | 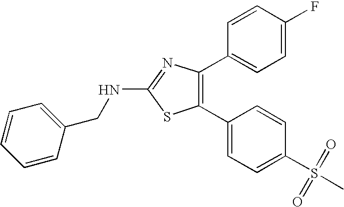 |
| | 4-(4-fluorophenyl)-5-(4-methylsulfonylphenyl)-2- |
| | benzylaminothiazole; |
| B-117 | 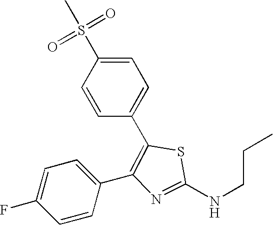 |
| | 4-(4-fluorophenyl)-5-(4-methylsulfonylphenyl)-2- |
| | (1-propylamino)thiazole; |
| B-118 | 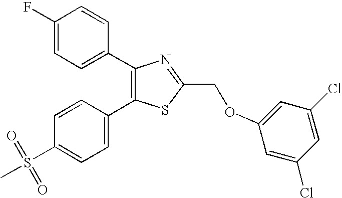 |
| | 2-((3,5-dichlorophenoxy)methyl)-4-(4-fluorophenyl)- |
| | 5-[4-(methylsulfonyl)phenyl]thiazole; |
| B-119 | 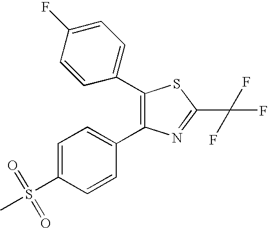 |
| | 5-(4-fluorophenyl)-4-(4-methylsulfonylphenyl)-2- |
| | trifluoromethylthiazole; |
| B-120 | 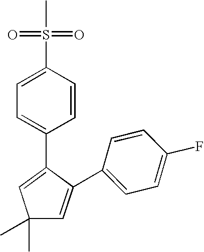 |
| | 1-methylsulfonyl-4-[1,1-dimethyl-4-(4-fluorophenyl) |
| | cyclopenta-2,4-dien-3-yl]benzene; |
| B-121 | 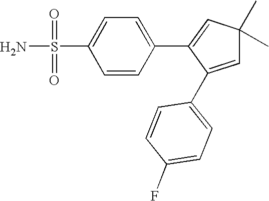 |
| | 4-[4-(4-fluorophenyl)-1,1-dimethylcyclopenta-2,4- |
| | dien-3-yl]benzenesulfonamide; |
| B-122 | 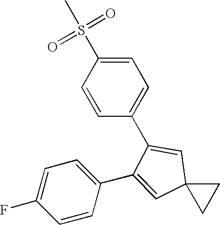 |
| | 5-(4-fluorophenyl)-6-[4-(methylsulfonyl)phenyl] |
| | spiro[2.4]hepta-4,6-diene; |
| B-123 | 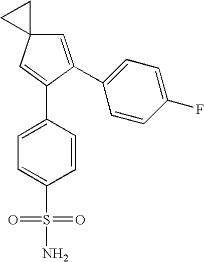 |
| | 4-[6-(4-fluorophenyl)spiro[2.4]hepta-4,6-dien- |
| | 5-yl]benzenesulfonamide; |
| B-124 | 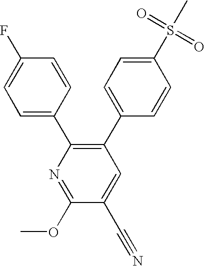 |
| | 6-(4-fluorophenyl)-2-methoxy-5-[4-(methylsulfonyl) |
| | phenyl]-pyridine-3-carbonitrile; |
| B-125 | 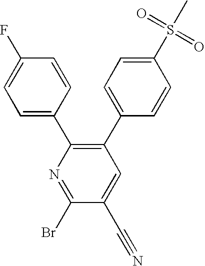 |
| | 2-bromo-6-(4-fluorophenyl)-5-[4-(methylsulfonyl)phenyl]- |
| | pyridine-3-carbonitrile; |
| B-126 | 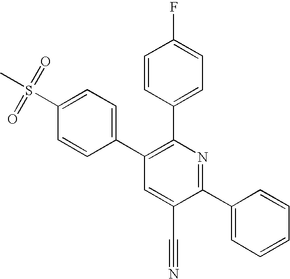 |
| | 6-(4-fluorophenyl)-5-[4-(methylsulfonyl)phenyl]-2- |
| | phenyl-pyridine-3-carbonitrile; |
| B-127 | 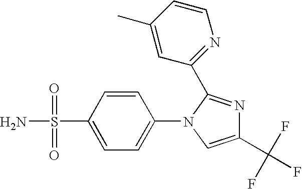 |
| | 4-[2-(4-methylpyridin-2-yl)-4-(trifluoromethyl)-1H- |
| | imidazol-1-yl]benzenesulfonamide; |
| B-128 | 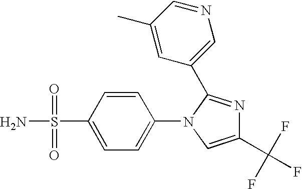 |
| | 4-[2-(5-methylpyridin-3-yl)-4-(trifluoromethyl)-1H- |
| | imidazol-1-yl]benzenesulfonamide; |
| B-129 | 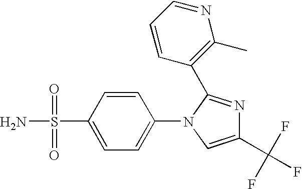 |
| | 4-[2-(2-methylpyridin-3-yl)-4-(trifluoromethyl)-1H- |
| | imidazol-1-yl]benzenesulfonamide; |
| B-130 | 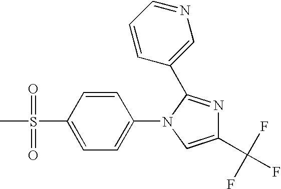 |
| | 3-[1-[4-(methylsulfonyl)phenyl]-4-(trifluoromethyl)- |
| | 1H-imidazol-2-yl]pyridine; |
| B-131 | 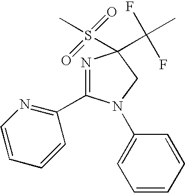 |
| | 2-[1-[4-(methylsulfonyl)phenyl-4-(trifluoromethyl)]- |
| | 1H-imidazol-2-yl]pyridine; |
| B-132 | 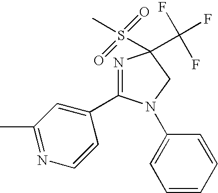 |
| | 2-methyl-4-[1-[4-(methylsulfonyl)phenyl-4- |
| | (trifluoromethyl)]-1H-imidazol-2-yl]pyridine; |
| B-133 | 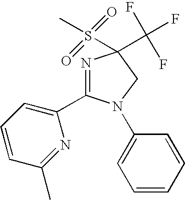 |
| | 2-methyl-6-[1-[4-(methylsulfonyl)phenyl-4- |
| | (trifluoromethyl)]-1H-imidazol-2-yl]pyridine; |
| B-134 | 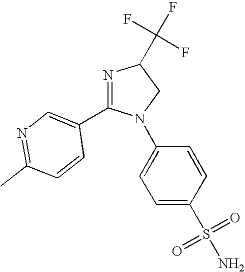 |
| | 4-[2-(6-methylpyridin-3-yl)-4-(trifluoromethyl)- |
| | 1H-imidazol-1-yl]benzenesulfonamide; |
| B-135 | 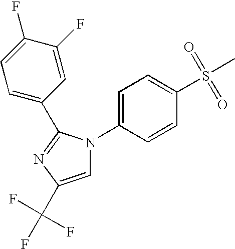 |
| | 2-(3,4-difluorophenyl)-1-[4-(methylsulfonyl)phenyl]- |
| | 4-(trifluoromethyl)-1H-imidazole; |
| B-136 | 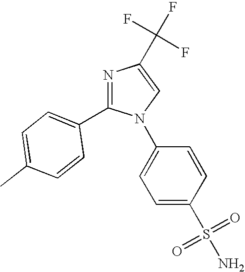 |
| | 4-[2-(4-methylphenyl)-4-(trifluoromethyl)-1H- |
| | imidazol-1-yl]benzenesulfonamide; |
| B-137 | 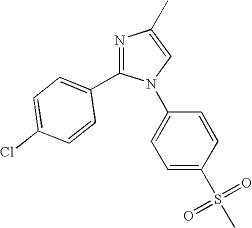 |
| | 2-(4-chlorophenyl)-1-[4-(methylsulfonyl)phenyl]-4- |
| | methyl-1H-imidazole; |
| B-138 | 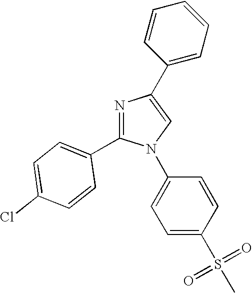 |
| | 2-(4-chlorophenyl)-1-[4-(methylsulfonyl)phenyl]-4- |
| | phenyl-1H-imidazole; |
| B-139 | 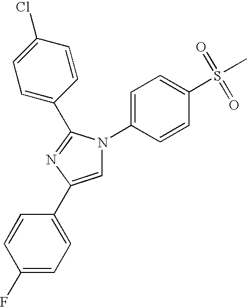 |
| | 2-(4-chlorophenyl)-4-(4-fluorophenyl)-1-[4- |
| | (methylsulfonyl)phenyl]-1H-imidazole; |
| B-140 | 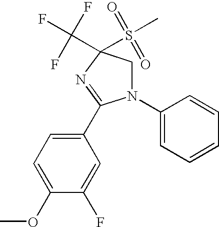 |
| | 2-(3-fluoro-4-methoxyphenyl)-1-[4-(methylsulfonyl)phenyl- |
| | 4-(trifluoromethyl)]-1H-imidazole; |
| B-141 | 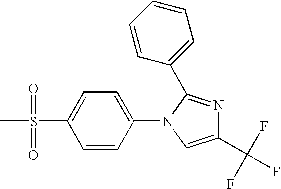 |
| | 1-[4-(methylsulfonyl)phenyl]-2-phenyl-4-trifluoromethyl- |
| | 1H-imidazole; |
| B-142 | 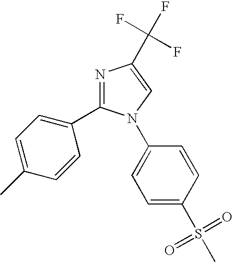 |
| | 2-(4-methylphenyl)-1-[4-(methylsulfonyl)phenyl]-4- |
| | trifluoromethyl-1H-imidazole; |
| B-143 | 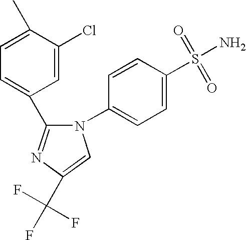 |
| | 4-[2-(3-chloro-4-methylphenyl)-4-(trifluoromethyl)- |
| | 1H-imidazol-1-yl]benzenesulfonamide; |
| B-144 | 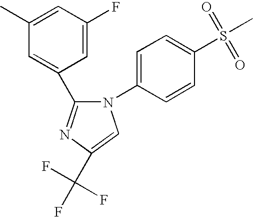 |
| | 2-(3-fluoro-5-methylphenyl)-1-[4-(methylsulfonyl)phenyl]- |
| | 4-(trifluoromethyl)-1H-imidazole; |
| B-145 | 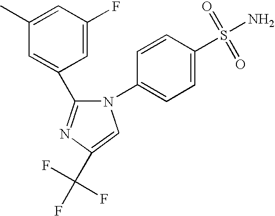 |
| | 4-[2-(3-fluoro-5-methylphenyl)-4-(trifluoromethyl- |
| | 1H-imidazole-1-yl]benzenesulfonamide; |
| B-146 | 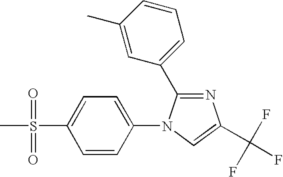 |
| | 2-(3-methylphenyl)-1-[4-(methylsulfonyl)phenyl]-4- |
| | trifluoromethyl-1H-imidazole; |
| B-147 | 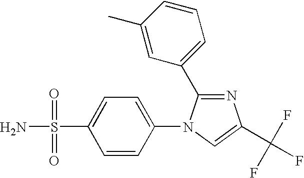 |
| | 4-[2-(3-methylphenyl)-4-trifluoromethyl-1H-imidazoi-1- |
| | yl]benzenesulfonamide; |
| B-148 | 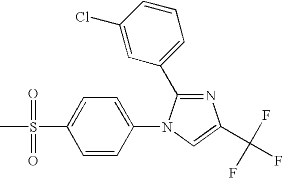 |
| | 1-[4-(methylsulfonyl)phenyl]-2-(3-chlorophenyl)- |
| | 4-trifluoromethyl-1H-imidazole |
| B-149 | 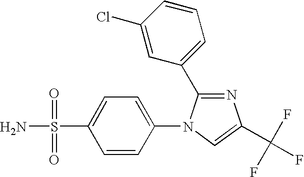 |
| | 4-[2-(3-chlorophenyl)-4-trifluoromethyl-1H- |
| | imidazol-1-yl]benzenesulfonamide; |
| B-150 | 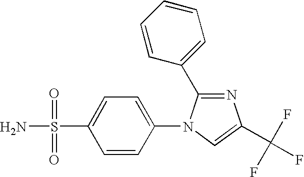 |
| | 4-[2-phenyl-4-trifluoromethyl-1H-imidazol-1- |
| | yl]benzenesulfonamide; |
| B-151 | 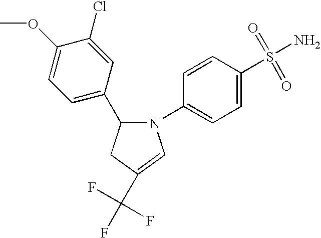 |
| | 4-[2-(4-methoxy-3-chlorophenyl)-4-trifluoromethyl- |
| | 1H-imidazol-1-yl]benzenesulfonamide; |
| B-152 | 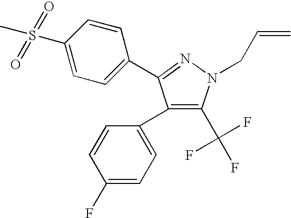 |
| | 1-allyl-4-(4-fluorophenyl)-3-[4-(methylsulfonyl)phenyl]- |
| | 5-(trifluoromethyl)-1H-pyrazole; |
| B-153 | 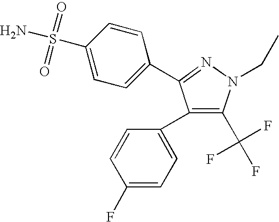 |
| | 4-[1-ethyl-4-(4-fluorophenyl)-5-(trifluoromethyl)-1H- |
| | pyrazol-3-yl]benzenesulfonamide; |
| B-154 | 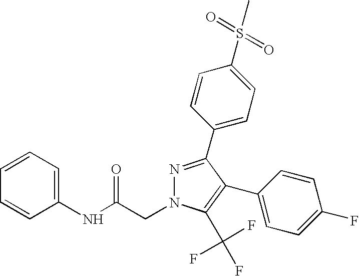 |
| | N-phenyl-[4-(4-fluorophenyl)-3-[4-(methylsulfonyl)phenyl]- |
| | 5-(trifluoromethyl)-1H-pyrazol-1-yl]acetamide; |
| B-155 | 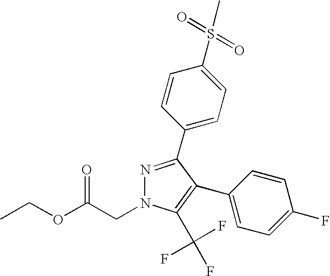 |
| | ethyl[4-(4-fluorophenyl)-3-[4-(methylsulfonyl)phenyl]- |
| | 5-(trifluoromethyl)-1H-pyrazol-1-yl]acetate; |
| B-156 | 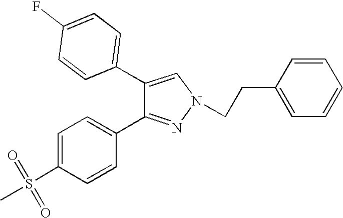 |
| | 4-(4-fluorophenyl)-3-[4-(methylsulfonyl)phenyl]-1-(2- |
| | phenylethyl)-1H-pyrazole; |
| B-157 | 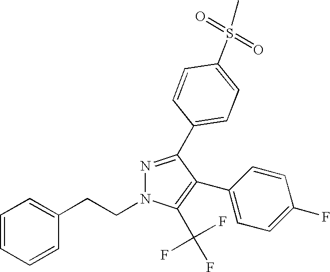 |
| | 4-(4-fluorophenyl)-3-[4-(methylsulfonyl)phenyl]- |
| | 1-(2-phenylethyl)-5-(trifluoromethyl)pyrazole; |
| B-158 | 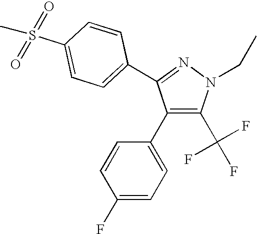 |
| | 1-ethyl-4-(4-fluorophenyl)-3-[4-methylsulfonyl)phenyl]- |
| | 5-(trifluoromethyl)-1H-pyrazole; |
| B-159 | 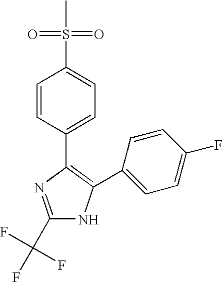 |
| | 5-(4-fluorophenyl)-4-(4-methylsulfonylphenyl)- |
| | 2-trifluoromethyl-1H-imidazole; |
| B-160 | 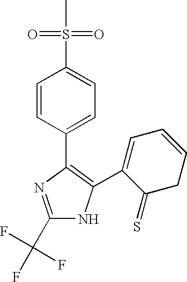 |
| | 4-[4-(methylsulfonyl)phenyl]-5-(2-thiophenyl)-2- |
| | (trifluoromethyl)-1H-imidazole; |
| B-161 | 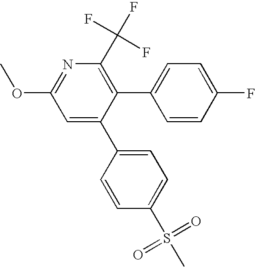 |
| | 5-(4-fluorophenyl)-2-methoxy-4-[4-(methylsulfonyl) |
| | phenyl]-6-(trifluoromethyl)pyridine; |
| B-162 | 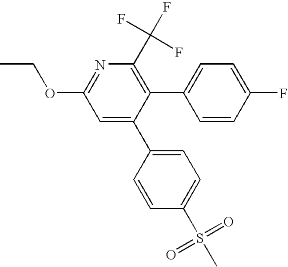 |
| | 2-ethoxy-5-(4-fluorophenyl)-4-[4-(methylsulfonyl)phenyl]- |
| | 6-(trifluoromethyl)pyridine; |
| B-163 | 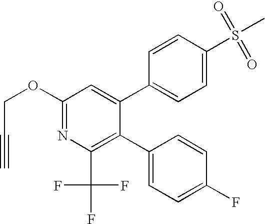 |
| | 5-(4-fluorophenyl)-4-[4-(methylsulfonyl)phenyl]-2- |
| | (2-propynyloxy)-6-(trifluoromethyl)pyridine; |
| B-164 | 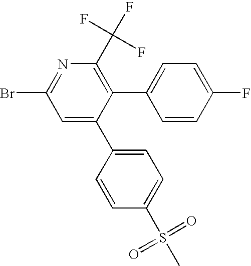 |
| | 2-bromo-5-(4-fluorophenyl)-4-[4-(methylsulfonyl)phenyl]- |
| | 6-(trifluoromethyl)pyridine; |
| B-165 | 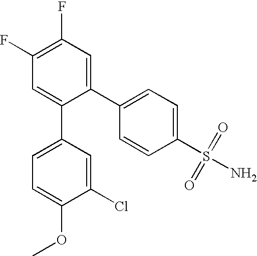 |
| | 4-[2-(3-chloro-4-methoxyphenyl)-4,5-difluorophenyl] |
| | benzenesulfonamide; |
| B-166 | 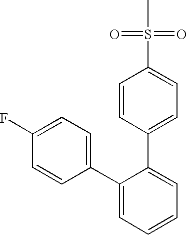 |
| | 1-(4-fluorophenyl)-2-[4-methylsulfonyl)phenyl]benzene; |
| B-167 | 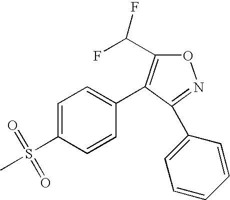 |
| | 5-difluoromethyl-4-(4-methylsulfonylphenyl)-3- |
| | phenylisoxazole; |
| B-168 | 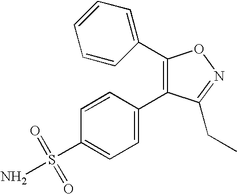 |
| | 4-[3-ethyl-5-phenylisoxazol-4-yl]benzenesulfonamide; |
| B-169 | 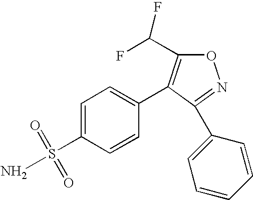 |
| | 4-[5-difluoromethyl-3-phenylisoxazol-4-yl] |
| | benzenesulfonamide; |
| B-170 | 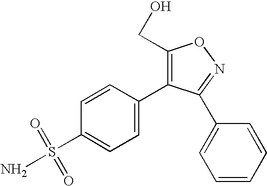 |
| | 4-[5-hydroxymethyl-3-phenylisoxazol-4-yl] |
| | benzenesulfonamide; |
| B-171 | 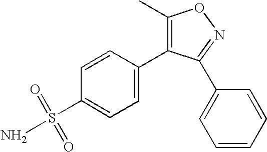 |
| | 4-[5-methyl-3-phenyl-isoxazol-4-yl]benzenesulfonamide; |
| B-172 | 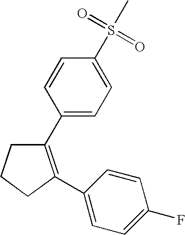 |
| | 1-[2-(4-fluorophenyl)cyclopenten-1-yl]-4- |
| | (methylsulfonyl)benzene; |
| B-173 | 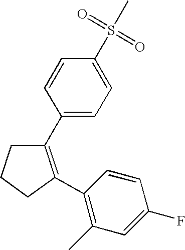 |
| | 1-[2-(4-fluoro-2-methylphenyl)cyclopenten-1-yl]-4- |
| | (methylsulfonyl)benzene; |
| B-174 | 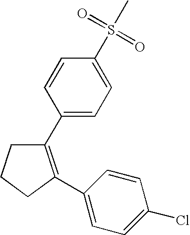 |
| | 1-[2-(4-chlorophenyl)cyclopenten-1-yl]-4- |
| | (methylsulfonyl)benzene; |
| B-175 | 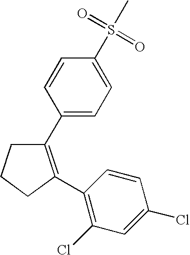 |
| | 1-[2-(2,4-dichlorophenyl)cyclopenten-1-yl]-4- |
| | (methylsulfonyl)benzene; |
| B-176 | 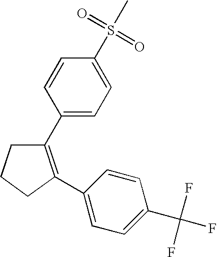 |
| | 1-[2-(4-trifloromethylphenyl)cyclopenten-1-yl]-4- |
| | (methylsulfonyl)benzene; |
| B-177 | 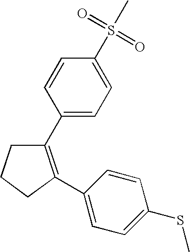 |
| | 1-[2-(4-methylthiophenyl)cyclopenten-1-yl]-4- |
| | (methylsulfonyl)benzene; |
| B-178 | 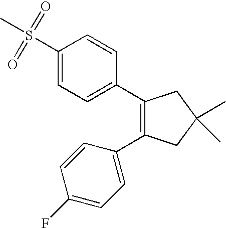 |
| | 1-[2-(4-fluorophenyl)-4,4-dimethylcyclopenten-1- |
| | yl]-4-(methylsulfonyl)benzene; |
| B-179 | 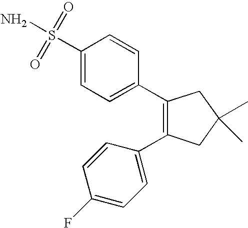 |
| | 4-[2-(4-fluorophenyl)-4,4-dimethylcyclopenten-1- |
| | yl]benzenesulfonamide; |
| B-180 | 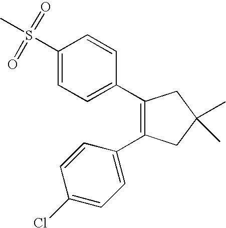 |
| | 1-[2-(3-chlorophenyl)-4,4-dimethylcyclopenten-1- |
| | yl]-4-(methylsulfonyl)benzene; |
| B-181 | 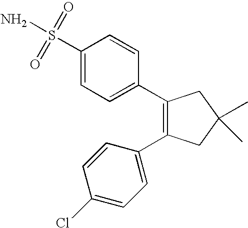 |
| | 4-[2-(4-chlorophenyl)-4,4-dimethylcyclopenten-1- |
| | yl]benzenesulfonamide; |
| B-182 | 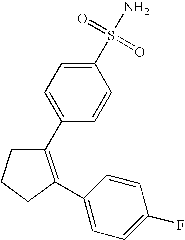 |
| | 4-[2-(4-fluorophenyl)cyclopenten-1- |
| | yl]benzenesulfonamide, |
| B-183 | 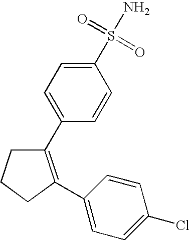 |
| | 4-[2-(4-chlorophenyl)cyclopenten-1- |
| | yl]benzenesulfonamide; |
| B-184 | 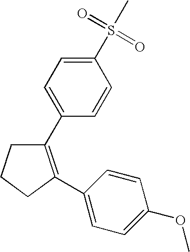 |
| | 1-[2-(4-methoxyphenyl)cyclopenten-1-yl]-4- |
| | (methylsulfonyl)benzene; |
| B-185 | 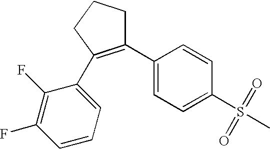 |
| | 1-[2-(2,3-difluorophenyl)cyclopenten-1-yl]-4- |
| | (methylsulfonyl)benzene; |
| B-186 | 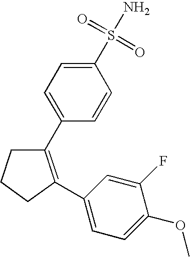 |
| | 4-[2-(3-fluoro-4-methoxyphenyl)cyclopenten-1- |
| | yl]benzenesulfonamide; |
| B-187 | 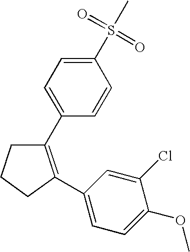 |
| | 1-[2-(3-chloro-4-methoxyphenyl)cyclopenten-1-yl]-4- |
| | (methylsulfonyl)benzene; |
| B-188 | 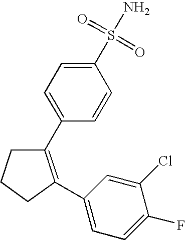 |
| | 4-[2-(3-chloro-4-fluorophenyl)cyclopenten-1- |
| | yl]benzenesulfonamide; |
| B-189 | 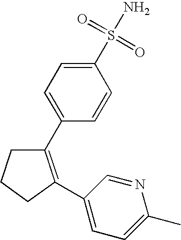 |
| | 4-[2-(2-methylpyridin-5-yl)cyclopenten-1-yl] |
| | benzenesulfonamide; |
| B-190 | 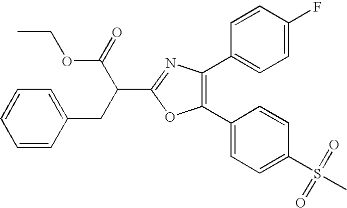 |
| | ethyl 2-[4-(4-fluorophenyl)-5-[4-(methylsulfonyl) |
| | phenyl]oxazol-2-yl]-2-benzyl-acetate; |
| B-191 | 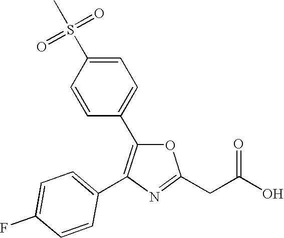 |
| | 2-[4-(4-fluorophenyl)-5-[4-(methylsulfonyl) |
| | phenyl]oxazol-2-yl]acetic acid; |
| B-192 | 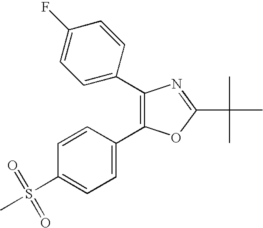 |
| | 2-(tert-butyl)-4-(4-fluorophenyl)-5-[4-(methylsulfonyl) |
| | phenyl]oxazole; |
| B-193 | 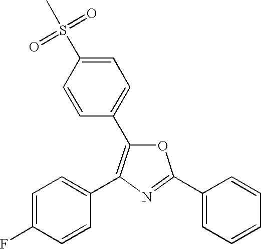 |
| | 4-(4-fluorophenyl)-5-[4-(methylsulfonyl)phenyl9 -2- |
| | phenyloxazole; |
| B-194 | 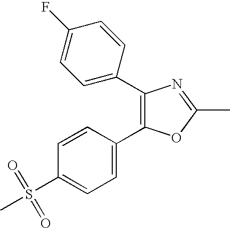 |
| | 4-(4-fluorophenyl)-2-methyl-5-[4-(methylsulfonyl) |
| | phenyl]oxazole; |
| B-195 | 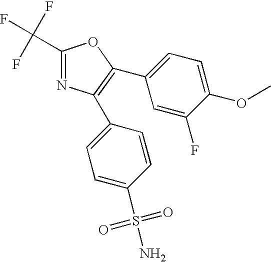 |
| | 4-[5-(3-fluoro-4-methoxyphenyl)-2-trifluoromethyl- |
| | 4-oxazolyl]benzenesulfonamide; |
| B-196 | 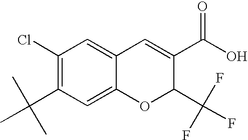 |
| | 6-chloro-7-(1,1-dimethylethyl)-2-trifluoromethyl-2H- |
| | 1-benzopyran-3-carboxylic acid; |
| B-197 | 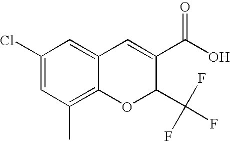 |
| | 6-chloro-8-methyl-2-trifluoromethyl-2H-1-benzopyran-3- |
| | carboxylic acid; |
| B-198 | 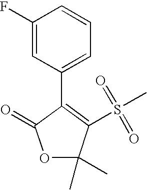 |
| | 5,5-dimethyl-3-(3-fluorophenyl)-4-methyl sulfonyl-2(5H)- |
| | furanone; |
| B-199 | 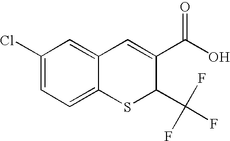 |
| | 6-chloro-2-trifluoromethyl-2N-1-benzothiopyran-3- |
| | carboxylic acid; |
| B-200 | 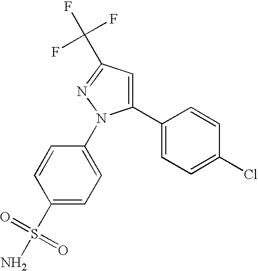 |
| | 4-[5-(4-chlorophenyl)-3-(trifluoromethyl)-1H-pyrazol- |
| | 1-yl]benzenesulfonamide; |
| B-201 | 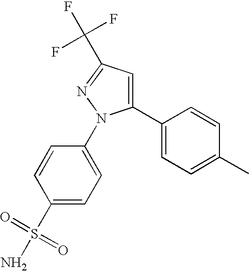 |
| | 4-[5-(4-methylphenyl)-3-(trifluoromethyl)-1H-pyrazol- |
| | 1-yl]benzenesulfonamide; |
| B-202 | 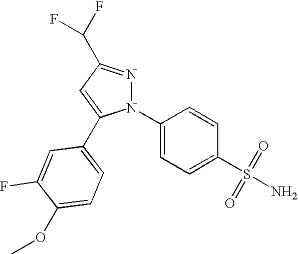 |
| | 4-[5-(3-fluoro-4-methoxyphenyl)-3-(difluoromethyl)- |
| | 1H-pyrazol-1-yl]benzenesulfonamide; |
| B-203 | 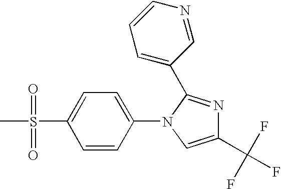 |
| | 3-[1-[4-(methylsulfonyl)phenyl]-4-trifluoromethyl- |
| | 1H-imidazol-2-yl]pyridine; |
| B-204 | 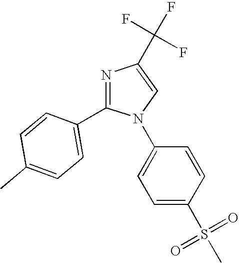 |
| | 2-methyl-5-[1-[4-(methylsulfonyl)phenyl]-4- |
| | trifluoromethyl-1H-imidazol-2-yl]pyridine; |
| B-205 | 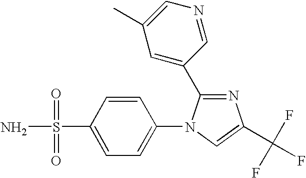 |
| | 4-[2-(5-methylpyridin-3-yl)-4-(trifluoromethyl)- |
| | 1H-imidazoI-1-yl]benzenesulfonamide; |
| B-206 | 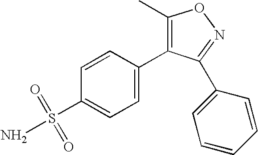 |
| | 4-(5-methyl-3-phenylisoxazol-4-yl]benzenesulfonamide; |
| B-207 | 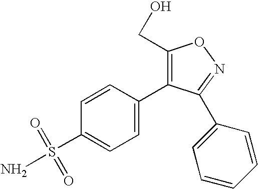 |
| | 4-[5-hydroxymethyl-3-phenylisoxazol-4-yl] |
| | benzenesulfonamide; |
| B-208 | 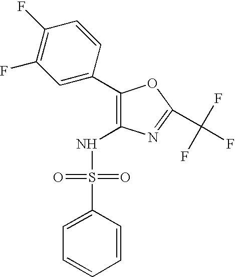 |
| | [2-trifluoromethyl-5-(3,4-difluorophenyl)-4- |
| | oxazolyl]benzenesulfonamide; |
| B-209 | 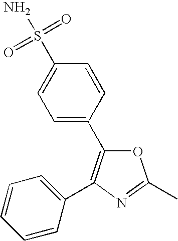 |
| | 4-[2-methyl-4-phenyl-5-oxazolyl]benzenesulfonamide; |
| B-210 | 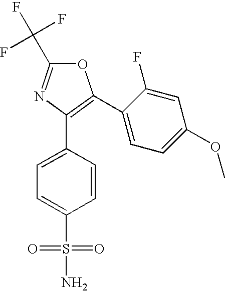 |
| | 4-[5-(2-fluoro-4-methoxyphenyl)-2-trifluoromethyl- |
| | 4-oxazolyl]benzenesulfonamide; |
| B-211 | 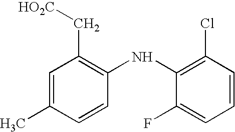 |
| B-212 | 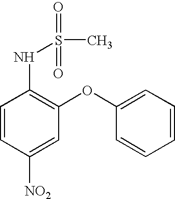 |
| | N-(4-nitro-2-phenoxy-phenyl)-methanesulfonamide or |
| | Nimesulide |
| B-213 | 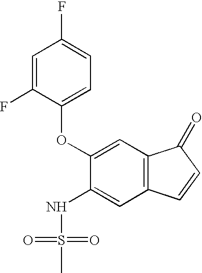 |
| | N-[6-(2,4-difluoro-phenoxy)-1-oxo-inden-5-yl]- |
| | methanesulfonamide or Flosulide |
| B-214 | 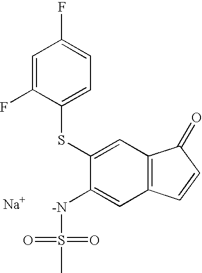 |
| | N-[6-(2,4-difluoro-phenylsulfanyl)-1-oxo-1H-inden- |
| | 5-yl]-methanesulfonamide, soldium salt, or L-745337 |
| B-215 | 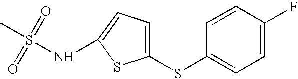 |
| | N-[5-(4-fluoro-phenylsulfanyl)-thiophen-2-yl]- |
| | methanesulfonamide or RWJ-63556 |
| B-216 | 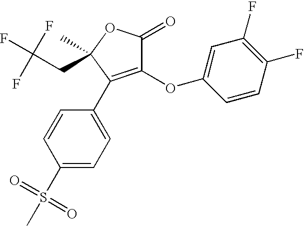 |
| | 3-(3,4-difluoro-phenoxy)-4-(4-methanesulfonyl-phenyl)-5- |
| | methyl-5-(2,2,2-trifluoro-ethyl)-5H-furan-2-one or |
| | L-784512 |
| B-217 | 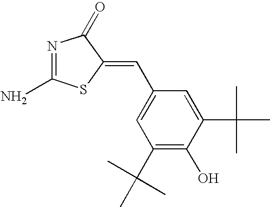 |
| | (5Z)-2-amino-5-[[3,5-bis(1,1-dimethylethyl)-4- |
| | hydroxyphenyl]methylene]-4(5H)-thiazolone or |
| | Darbufelone |
| B-218 | CS-502 |
| B-219 | LAS-34475 |
| B-220 | LAS-34555 |
| B-221 | S-33516 |
| B-222 | SD-8381 |
| B-223 | L-783003 |
| B-224 | 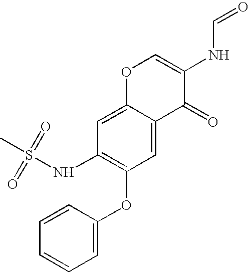 |
| | N-[3-(formylamino)-4-oxo-6-phenoxy-4H-1-benzopyran- |
| | 7-yl]-methanesulfonamide or T614 |
| B-225 | D-1367 |
| B-226 | L-748731 |
| B-227 | 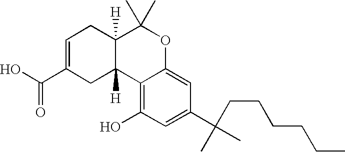 |
| | (6aR,10aR)-3-(1,1-dimethylheptyl)-6a,7,10,10a-tetrahydro- |
| | 1-hydroxy-6,6-dimethyl-6H-dibenzo[b,d]pyran-9- |
| | carboxylic acid or CT3 |
| B-228 | CGP-28238 |
| B-229 | 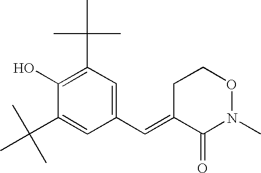 |
| | 4-[[3,5-bis(1,1-dimethylethyl)-4-hydroxyphenyl] |
| | methylene]dihydro-2-methyl-2H-1,2-oxazin-3(4H)- |
| | one or BF-389 |
| B-230 | GR-253035 |
| B-231 | 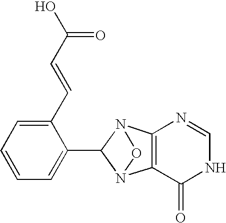 |
| | 2-(6-dioxo-9H-purin-8-yl)cinnamic acid |
| B-232 | S-2474 |
| B-233 | 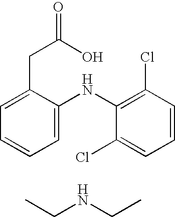 |

The cyclooxygenase-2 selective inhibitors utilized in the present invention may be in the form of free bases or pharmaceutically acceptable acid addition salts thereof. The term “pharmaceutically-acceptable salts” embraces salts commonly used to form alkali metal salts and to form addition salts of free acids or free bases. The nature of the salt may vary, provided that it is pharmaceutically-acceptable. Suitable pharmaceutically-acceptable acid addition salts of compounds for use in the present methods may be prepared from an inorganic acid or from an organic acid. Examples of such inorganic acids are hydrochloric, hydrobromic, hydroiodic, nitric, carbonic, sulfuric and phosphoric acid. Appropriate organic acids may be selected from aliphatic, cycloaliphatic, aromatic, araliphatic, heterocyclic, carboxylic and sulfonic classes of organic acids, examples of which are formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, 4-hydroxybenzoic, phenylacetic, mandelic, embonic (pamoic), methanesulfonic, ethanesulfonic, benzenesulfonic, pantothenic, 2-hydroxyethanesulfonic, toluenesulfonic, sulfanilic, cyclohexylaminosulfonic, stearic, algenic, β-hydroxybutyric, salicylic, galactaric and galacturonic acid. Suitable pharmaceutically-acceptable base addition salts of compounds of use in the present methods include metallic salts made from aluminum, calcium, lithium, magnesium, potassium, sodium and zinc or organic salts made from N,N′-dibenzylethylenediamine, chloroprocaine, choline, diethanolamine, ethylenediamine, meglumine (N-methylglucamine) and procaine. All of these salts may be prepared by conventional means from the corresponding compound by reacting, for example, the appropriate acid or base with the compound of any Formula set forth herein.

The cyclooxygenase-2 selective inhibitors useful in the practice of the present invention can be formulated into pharmaceutical compositions and administered by any means that will deliver a therapeutically effective dose. Such compositions can be administered orally, parenterally, by inhalation spray, rectally, intradermally, transdermally, or topically in dosage unit formulations containing conventional nontoxic pharmaceutically acceptable carriers, adjuvants, and vehicles as desired. Topical administration may also involve the use of transdermal administration such as transdermal patches or iontophoresis devices. The term parenteral as used herein includes subcutaneous, intravenous, intramuscular, or intrasternal injection, or infusion techniques. Formulation of drugs is discussed in, for example, Hoover, John E., Remington's Pharmaceutical Sciences, Mack Publishing Co., Easton, Pa. (1975), and Liberman, H. A. and Lachman, L., Eds., Pharmaceutical Dosage Forms, Marcel Decker, New York, N.Y. (1980).

Injectable preparations, for example, sterile injectable aqueous or oleaginous suspensions, can be formulated according to the known art using suitable dispersing or wetting agents and suspending agents. The sterile injectable preparation may also be a sterile injectable solution or suspension in a nontoxic parenterally acceptable diluent or solvent. Among the acceptable vehicles and solvents that may be employed are water, Ringer's solution, and isotonic sodium chloride solution. In addition, sterile, fixed oils are conventionally employed as a solvent or suspending medium. For this purpose, any bland fixed oil may be employed, including synthetic mono- or diglycerides. In addition, fatty acids such as oleic acid are useful in the preparation of injectables. Dimethyl acetamide, surfactants including ionic and non-ionic detergents, and polyethylene glycols can be used. Mixtures of solvents and wetting agents such as those discussed above are also useful.

Suppositories for rectal administration of the compounds discussed herein can be prepared by mixing the active agent with a suitable non-irritating excipient such as cocoa butter, synthetic mono-, di-, or triglycerides, fatty acids, or polyethylene glycols which are solid at ordinary temperatures but liquid at the rectal temperature, and which will therefore melt in the rectum and release the drug.

Solid dosage forms for oral administration may include capsules, tablets, pills, powders, and granules. In such solid dosage forms, the compounds are ordinarily combined with one or more adjuvants appropriate to the indicated route of administration. If administered per os, the compounds can be admixed with lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, and/or polyvinyl alcohol, and then tableted or encapsulated for convenient administration. Such capsules or tablets can contain a controlled-release formulation as can be provided in a dispersion of active compound in hydroxypropylmethyl cellulose. In the case of capsules, tablets, and pills, the dosage forms can also comprise buffering agents such as sodium citrate, or magnesium or calcium carbonate or bicarbonate. Tablets and pills can additionally be prepared with enteric coatings.

For therapeutic purposes, formulations for parenteral administration can be in the form of aqueous or non-aqueous isotonic sterile injection solutions or suspensions. These solutions and suspensions can be prepared from sterile powders or granules having one or more of the carriers or diluents mentioned for use in the formulations for oral administration. The compounds can be dissolved in water, polyethylene glycol, propylene glycol, ethanol, corn oil, cottonseed oil, peanut oil, sesame oil, benzyl alcohol, sodium chloride, and/or various buffers. Other adjuvants and modes of administration are well and widely known in the pharmaceutical art.

Liquid dosage forms for oral administration can include pharmaceutically acceptable emulsions, solutions, suspensions, syrups, and elixirs containing inert diluents commonly used in the art, such as water. Such compositions can also comprise adjuvants, such as wetting agents, emulsifying and suspending agents, and sweetening, flavoring, and perfuming agents.

The amount of active ingredient that can be combined with the carrier materials to produce a single dosage of the cyclooxygenase-2 selective inhibitor will vary depending upon the patient and the particular mode of administration. In general, the pharmaceutical compositions may contain a cyclooxygenase-2 selective inhibitor in the range of about 0.1 to 2000 mg, preferably in the range of about 0.5 to 500 mg and most preferably between about 1 and 200 mg. A daily dose of about 0.01 to 100 mg/kg body weight, preferably between about 0.1 and about 50 mg/kg body weight and most preferably from about 1 to 20 mg/kg body weight, may be appropriate. The daily dose can be administered in one to four doses per day.

In one embodiment, when the cyclooxygenase-2 selective inhibitor comprises rofecoxib, it is preferred that the amount used is within a range of from about 0.15 to about 1.0 mg/day·kg, and even more preferably from about 0.18 to about 0.4 mg/day·kg.

In still another embodiment, when the cyclooxygenase-2 selective inhibitor comprises etoricoxib, it is preferred that the amount used is within a range of from about 0.5 to about 5 mg/day·kg, and even more preferably from about 0.8 to about 4 mg/day·kg.

Further, when the cyclooxygenase-2 selective inhibitor comprises celecoxib, it is preferred that the amount used is within a range of from about 1 to about 20 mg/day·kg, even more preferably from about 1.4 to about 8.6 mg/day·kg, and yet more preferably from about 2 to about 3 mg/day·kg.

When the cyclooxygenase-2 selective inhibitor comprises valdecoxib, it is preferred that the amount used is within a range of from about 0.1 to about 5 mg/day·kg, and even more preferably from about 0.8 to about 4 mg/day·kg.

In a further embodiment, when the cyclooxygenase-2 selective inhibitor comprises parecoxib, it is preferred that the amount used is within a range of from about 0.1 to about 5 mg/day·kg, and even more preferably from about 1 to about 3 mg/day·kg.

Those skilled in the art will appreciate that dosages may also be determined with guidance from Goodman & Goldman's The Pharmacological Basis of Therapeutics, Ninth Edition (1996), Appendix II, pp. 1707-1711 and from Goodman & Goldman's The Pharmacological Basis of Therapeutics, Tenth Edition (2001), Appendix II, pp. 475-493.

Amyloid Beta Vaccines

In addition to a cyclooxygenase-2 selective inhibitor, the combination therapy of the present invention also comprises an amyloid beta vaccine, wherein the vaccine comprises at least one Abeta peptide that is generally deposited in amyloid plaques, or a fragment, analog or variant thereof. While not being bound to a particular theory, the amyloid beta vaccines of the present invention appear to exhibit therapeutic effects due to their immunogenicity and resulting production of antibodies. These antibodies are believed to bind to soluble amyloid peptides and neutralize them before they deposit into amyliod plaques and/or bind to already-formed plaques and assist in their removal.

For preparation of amyloid beta vaccines, different isoforms of the amyloid beta peptide are used. Furthermore, the vaccine can comprise fragments, variants, or analogs of Abeta. The amyloid peptides that can be used in vaccine preparation include but are not limited to: Abeta (1-42), Abeta (1-43), Abeta (1-40), Abeta (1-39), and Abeta (1-41). Furthermore, the fragments of Abeta that can be used include but are not limited to: Abeta (1-28), Abeta (1-16), Abeta (25-35), Abeta (29-39), Abeta (29-40), Abeta (29-41), Abeta (29-42), Abeta (29-43), Abeta (26-42), Abeta (26-43), and Abeta (35-43). In a preferred embodiment, the amyloid beta peptide used to prepare an amyloid vaccine of the present invention comprises Abeta (1-42).

For the purposes of the present invention, the vaccine can be either monovalent (consisting of only one antigen) or multivalent (containing more than one antigen), wherein the antigen refers to Abeta peptide or a fragment, variant or analog thereof. Accordingly, the monovalent vaccine of the present invention comprises one Abeta peptide or one Abeta fragment, variant or analog thereof whereas the multivalent vaccine comprises at least two isoforms of Abeta peptides, or at least two Abeta fragments, variants or analogs, or a combination thereof. By way of example, the monovalent vaccine comprises Abeta peptide (1-42) or Abeta fragment (25-35), whereas the multivalent vaccine comprises, e.g., 1) Abeta (1-42) and Abeta (1-40), or 2) Abeta (1-42) and Abeta (25-35), or 3) Abeta (25-35) and Abeta (1-28).

In an alternative embodiment, the vaccines of the present invention may be prepared from the amyloid beta peptide nucleic acid sequences and/or suitable vectors containing said nucleotide sequences. Similarly to peptide vaccines, it is believed that the nucleic acid vaccines elicit an immune response in a subject, wherein the response includes production of anti-amyloid beta antibodies.

Peptide Synthesis

Skilled artisans will recognize that the amyloid beta peptides of the present invention and fragments, variant and analogs thereof can be synthesized by a number of different methods. All of the amino acid compounds of the invention can be made by chemical methods well known in the art, including, e.g., solid phase peptide synthesis and recombinant methods. Both methods are described, for instance, in U.S. Pat. No. 4,617,149.

Furthermore, the principles of solid phase chemical synthesis of polypeptides are well known in the art and may be found in general texts in the area. See, e.g., H. Dugas and C. Penney, BIOORGANIC CHEMISTRY, (1981) Springer-Verlag, New York, pgs. 54-92. For example, peptides may be synthesized by solid-phase methodology utilizing an Applied Biosystems 430A peptide synthesizer (commercially available from Applied Biosystems, Foster City Calif.) and synthesis cycles supplied by Applied Biosystems. Protected amino acids, such as t-butoxycarbonyl-protected amino acids, and other reagents are commercially available from many chemical supply houses. By way of example, Fraser et al. manuscript describes the procedure for synthesizing Abeta peptides and fragments thereof using FMOC solid phase procedure (J Neurosci Res, 28(4):474-485, 1991).

Recombinant Peptides

In addition, the DNA sequences encoding the amyloid beta peptides or fragments, analogs or variants thereof can be produced. The synthesis of nucleic acids is well known in the art. See, e.g., E. L. Brown, R. Belagaje, M. J. Ryan, and H. G. Khorana, Methods in Enzymology, 68:109-151 (1979). The DNA segments corresponding to the amyloid beta peptides or fragments thereof can be generated using conventional DNA synthesizing apparatus such as the Applied Biosystems Model 380A or 380B DNA synthesizers (commercially available from Applied Biosystems, Inc., 850 Lincoln Center Drive, Foster City, Calif. 94404) which employ phosphoramidite chemistry. In the alternative, the more traditional phosphotriester chemistry may be employed to synthesize the nucleic acids of this invention. See, e.g., OLIGONUCLEOTIDE SYNTHESIS, A PRACTICAL APPROACH, (M. J. Gait, ed., 1984).

Following the synthesis of DNA sequences, such sequences are produced by utilizing recombinant systems. The basic steps in the recombinant production of desired peptides are: integrating said DNA into an expression vector in a manner suitable for the expression of the peptide of interest, either alone or as a fusion protein; transforming an appropriate eukaryotic or prokaryotic host cell with said expression vector; culturing said transformed or transfected host cell in a manner to express the peptide of interest; and recovering and purifying the recombinantly produced peptide of interest.

The methods of recombinantly producing peptides/proteins are well known in the art. Literature that describes these techniques includes, for example, Sambrook, et al., Molecular Cloning: A Laboratory Manual, Cold Spring Harbor Laboratory, Cold Spring Harbor, N.Y. (2nd edition, 1989); Ausubel, et al., Current Protocols in Molecular Biology (1987); O'Reilly, et al., Baculovirus Expression Vectors: A Laboratory Manual (1992); Practical Molecular Virology (Collins, ed., 1991); Culture of Animal Cells: A Manual of Basic Technique (Freshney, ed., 2nd edition, 1989); J. Miller, Experiments in Molecular Genetics, Cold Spring Harbor Laboratory, Cold Spring Harbor, N.Y. (1972); D. A. Morrison, Transformation and Preservation of Competent Bacterial Cells by Freezing, Methods Enzymol. 68:326-331 (1979); and J. Perbal, A Practical Guide to Molecular Cloning, John Wiley & Sons (1984).

Peptide Purification

After the desired peptide is obtained either by chemical synthesis or recombinant methods, it can be isolated and purified using a number of procedures that are well known in the art, such as, e.g., extraction, precipitation, chromatography, affinity chromatography, electrophoresis, or the like. For example, purification of amyloid beta peptides following FMOC synthesis by high pressure liquid chromatography (HPLC) is described in Fraser et al. (J Neurosci Res, 28(4):474-485, 1991).

Preparation and Administration of Vaccines

Immunogenic vaccines of the present invention may be administered parenterally, such as by injection subcutaneously, intramuscularly, intradermally, intraperitoneally, or intravenously. Alternatively, other modes of administration including suppositories and oral formulations may be desirable. The one or more amyloid beta peptides and/or fragments, analogs or variants thereof may be mixed with pharmaceutically acceptable excipients or carriers, which are compatible therewith. Such excipients may include, water, saline, dextrose, glycerol, ethanol, and combinations thereof. For suppositories, binders and carriers may include, for example, polyalkalene glycols or triglycerides. Oral formulations may include normally employed incipients such as, for example, pharmaceutical grades of saccharine, cellulose and magnesium carbonate. These compositions can take the form of solutions, suspensions, tablets, pills, capsules, sustained release formulations or powders and contain about 1 to 95% of the amyloid beta peptide or fragment, analog, or variant thereof.

The immunogenic vaccines may further contain auxiliary substances, such as wetting or emulsifying agents, pH buffering agents, or adjuvants to enhance the effectiveness thereof. Vaccine preparation is generally described in New Trends and Developments in Vaccines, edited by Voller et al., University Park Press, Baltimore, Md., U.S.A. 1978 and Remington's Pharmaceutical Science; Mack Publishing Company Easton, Pa. (latest edition).

Immunogenicity can be significantly improved if the antigens are co-administered with adjuvants, commonly used as 0.05 to 0.1 percent solutions in phosphate-buffered saline. Adjuvants enhance the immunogenicity of an antigen but are not necessarily immunogenic themselves. Adjuvants may act by retaining the antigen locally near the site of administration to produce a depot effect facilitating a slow, sustained release of antigen to cells of the immune system. Adjuvants can also attract cells of the immune system to an antigen depot and stimulate such cells to elicit immune responses. Intrinsic adjuvants, such as lipopolysaccharides, are generally the components of the killed or attenuated bacteria used as vaccines. Extrinsic adjuvants are immunomodulators which are typically non-covalently linked to antigens and are formulated to enhance the host immune responses.

Desirable characteristics of ideal adjuvants include: lack of toxicity; ability to stimulate a long-lasting immune response; simplicity of manufacture and stability in long-term storage; ability to elicit the desirable response to antigens administered by various routes, (e.g. for the treatment of Alzheimer's disease, production of antibodies that are able to bind to and neutralize/clear amyloid beta peptides is desirable); synergy with other adjuvants; capability of selectively interacting with populations of antigen presenting cells (APC); and the ability to selectively increase appropriate antibody isotype levels (for example, IgG) against antigens.

Accordingly, the vaccines of the present invention may be formulated with various adjuvants or immunomodulating agents including, for example, aluminum hydroxide, aluminum phosphate, aluminum potassium sulfate (alum), beryllium sulfate, silica, kaolin, carbon, water-in-oil emulsions, oil-in-water emulsions, muramyl dipeptide, bacterial endotoxin, lipid X, Corynebacterium parvum (Propionibacterium acnes), Bordetella pertussis, polyribonucleotides, sodium alginate, lanolin, lysolecithin, vitamin A, saponin, liposomes, levamisole, DEAE-dextran, blocked copolymers or other synthetic adjuvants. Such adjuvants are available commercially from various sources, for example, Merck Adjuvant 65 (Merck and Company, Inc., Rahway, N.J.). Adjuvants, including liposomes, are discussed in the following references, e.g., :Gregoriades, G. et al., Immunological Adjuvants and Vaccines, Plenum Press, New York, 1989 Michalek, S. M. et al., “Liposomes as Oral Adjuvants,” Curr. Top. Microbiol. Immunol. 146:51-58 (1989).

Aluminum hydroxide and aluminum phosphate (collectively commonly referred to as alum) are routinely used as adjuvants in human and veterinary vaccines. For example, the efficacy of alum in increasing antibody responses to diphtheria and tetanus toxoids is well established. Thus, in a preferred embodiment, the adjuvant used to produce amyloid beta vaccines of the present invention comprises aluminum hydroxide or aluminum phosphate.

In another embodiment, oil in water emulsions per se are well known in the art, and have been suggested to be useful as adjuvant compositions (see, e.g., EPO 399843). In order for any oil in water composition to be suitable for human administration, the oil phase of the emulsion system has to comprise a metabolizable oil, that is, an oil “capable of being transformed by metabolism” (Dorland's Illustrated Medical Dictionary, W. B. Sanders Company, 25th edition (1974)). The oil may be any vegetable oil, fish oil, animal oil or synthetic oil, which is not toxic to the recipient and is capable of being transformed by metabolism. Nuts, seeds, and grains are common sources of vegetable oils. Synthetic oils are also part of this invention and can include commercially available oils.

For formulation of amyloid beta nucleic acid vaccines, the vaccines may be prepared as injectables, in physiologically-acceptable liquid solutions or emulsions for polynucleotide administration. The nucleic acid may be associated with liposomes, such as lecithin liposomes or other liposomes known in the art or the nucleic acid may be associated with an adjuvant, as previously described. Liposomes comprising cationic lipids interact spontaneously and rapidly with polyanions, such as DNA and RNA, resulting in liposome/nucleic acid complexes that capture up to 100% of the polynucleotide. In addition, the polycationic complexes fuse with cell membranes, resulting in an intracellular delivery of polynucleotide that bypasses the degradative enzymes of the lysosomal compartment. PCT application WO 94/27435 describes compositions for genetic immunization comprising cationic lipids and polynucleotides. Furthermore, in order to assist the cellular uptake of nucleic acid, agents, such as calcium ions, viral proteins and other transfection facilitating agents, may be advantageously used.

The immunogenic vaccines of the present invention are administered in a manner compatible with the dosage formulation, and in such amount as will be therapeutically effective, protective and immunogenic. The quantity to be administered depends on the subject to be treated, including, for example, the capacity of the individual's immune system to synthesize antibodies, and if needed, to produce a cell-mediated immune response. Precise amounts of active ingredient required to be administered depend on the judgment of the practitioner. However, suitable dosage ranges are readily determinable by one skilled in the art and may be of the order of micrograms of the amyloid beta peptides or fragments thereof. Suitable regimes for initial administration and booster doses are also variable, but may include an initial administration followed by subsequent administrations. The dosage may also depend on the route of administration and will vary according to the size of the host.

Generally, it is expected that each dose will comprise the amyloid beta peptide(s) in the amount between about 0.01 μg/kg body weight and about 1000 μg/kg body weight of the subject. Preferably, each dose will be about 500 μg/kg body weight of the peptide(s), and more preferably about 300 μg/kg body weight of the amyloid beta peptide(s). An optimal amount for a particular vaccine can be ascertained by standard studies involving observation of appropriate immune responses in subjects. Following an initial vaccination, subjects may receive one or several booster immunizations adequately spaced, for example after 2 and 6 months. In another embodiment, an amyloid beta vaccine may be administered to a subject at regularly spaced intervals, for example once/6 months. In addition, the vaccine may be administerd to a subject at regularly spaced intervals for the life of the subject.

With respect to the Cox-2 inhibitor administration, the initial amyloid beta vaccine may be administered prior to the start of a Cox-2 inhibitor administration. Other options include administering a Cox-2 inhibitor prior to the initial amyloid vaccination or administering it during the time intervals between each vaccination.

Other embodiments within the scope of the embodiments herein will be apparent to one skilled in the art from consideration of the specification or practice of the invention as disclosed herein. It is intended that the specification be considered to be exemplary only, with the scope and spirit of the invention being indicated by the embodiments.

All references cited in this specification, including without limitation, all papers, publications, patents, patent applications, presentations, texts, reports, manuscripts, brochures, books, internet postings, journal articles, periodicals, and the like, are hereby incorporated by reference into this specification in their entireties.

As various changes could be made in the above methods and compositions without departing from the scope of the invention, it is intended that all matter contained in this application shall be interpreted as illustrative and not in a limiting sense.

### EXAMPLES

The following examples are intended to provide illustrations of the application of the present invention. The following examples are not intended to completely define or otherwise limit the scope of the invention.

### Example 1

### Mouse Model of Alzheimer's Disease

PDAPP mice, transgenic for an amyloid β precursor protein (APP) mini-gene driven by a platelet-derived (PD) growth factor promoter, overexpress one of the disease-linked mutant forms of the human APP protein, and as a result exhibit many of the pathological features of Alzheimer's disease including deposition of extracellular amyloid plaques (Games et al., Nature, 373, pp.523-527, 1995). Accordingly, these mice provide a suitable model system for determining the effect of different treatments on Alzheimer's disease.

Non-transgenic mice (healthy control mice), non-transgenic mice receiving a placebo treatment, PDAPP mice receiving no treatment, and PDAPP mice receiving a combination of Cox-2 inhibitor and amyloid immunizations are used to assess the efficacy of the treatment. Non-transgenic mice are preferably of the same genetic background as PDAPP mice.

For the experiment, combinations of different Cox-2 inhibitors and different amyloid beta vaccines are tested. For example and without limitation, celecoxib is tested in combination with Abeta (1-42)-comprising vaccine or in combination with Abeta (1-28) vaccine, and rofecoxib is tested with either of the two vaccines. However, it should be noted that any Cox-2 inhibitor described herein could be tested in combination with any of the amyloid beta vaccines described herein. Furthermore, for each combination of a Cox-2 inhibitor and amyloid beta vaccine, several different doses of Cox-2 inhibitor should be tested with several doses of amyloid beta peptides contained in the vaccines to test the efficacy of the treatment.

The results of the treatment can be determined through a number of different tests. For example, a behavioral test, such as a radial-arm maze or water maze, can be used to compare the abilities of treated mice versus control mice. Specifically, deleterious behavior, such as confusion and failure of memory, can be evaluated based upon observation of the performance of mice in such tests.

Additionally, numerous epidemiological tests can be performed to determine the amount of swelling in the brain tissue, the amount of amyloid plaque deposit and neurofibrillary tangle deposit in the brain, and the amount of bound Aβ found in the plasma and cerebrospinal fluid. The methods for measuring the above-mentioned characteristics are well known in the art. See, for instance, Bard et al., Nature Medicine, Vol. 6, no. 8, pp.916-919, August 2000 and Morgan et al., Nature, Vol. 408, pp. 982-985, 21/28 December 2000).

What is claimed is:

1. A method for the treatment or prevention of Alzheimer's disease in a subject, the method comprising administering to the subject a cyclooxygenase-2 selective inhibitor or a pharmaceutically acceptable salt or prodrug thereof and an amyloid beta vaccine.

2. The method of claim 1 wherein the cyclooxygenase-2 selective inhibitor comprises a chromene compound.

3. The method of claim 2 wherein the chromene compound is a benzopyran or substituted benzopyran analog.

4. The method of claim 3 wherein the benzopyran or substituted benzopyran analog is selected from the group consisting of benzothiopyrans, dihydroquinolines and dihydronaphthalenes.

5. The method of claim 1 wherein the cyclooxygenase-2 selective inhibitor comprises a tricyclic compound.

6. The method of claim 5 wherein the tricyclic compound comprises a benzenesulfonamide or methylsulfonylbenzene.

7. The method of claim 1 wherein the cyclooxygenase-2 selective inhibitor comprises a phenyl acetic acid derivative.

8. The method of claim 1 wherein the cyclooxygenase-2 selective inhibitor comprises: 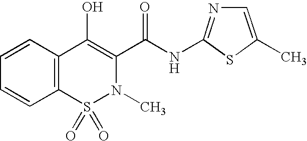
or pharmaceutically acceptable salt or prodrug thereof.

9. The method of claim 1 wherein the cyclooxygenase-2 selective inhibitor comprises: 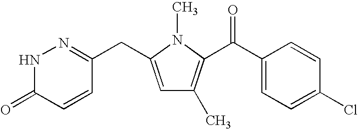
or a pharmaceutically acceptable salt or prodrug thereof.

10. The method of claim 1 wherein the cyclooxygenase-2 selective inhibitor comprises a compound of the formula: 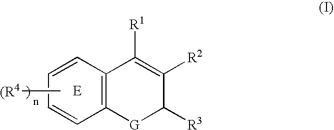
wherein n is an integer which is 0, 1, 2, 3 or 4;
wherein G is O, S or NR^{a};
wherein R^{a} is alkyl;
wherein R¹ is selected from the group consisting of H and aryl;
wherein R² is selected from the group consisting of carboxyl, aminocarbonyl, alkylsulfonylaminocarbonyl and alkoxycarbonyl;
wherein R³ is selected from the group consisting of haloalkyl, alkyl aralkyl, cycloalkyl and aryl optionally substituted with one or more radicals selected from alkylthio, nitro and alkylsulfonyl; and
wherein each R⁴ is independently selected from the group consisting of H, halo, alkyl, aralkyl, alkoxy, aryloxy, heteroaryloxy, aralkyloxy, heteroaralkyloxy, haloalkyl, haloalkoxy, alkylamino, arylamino, aralkylamino, heteroarylamino, heteroarylalkylamino, nitro, amino, aminosulfonyl, alkylaminosulfonyl, arylaminosulfonyl, heteroarylaminosulfonyl, aralkylaminosulfonyl, heteroaralkylaminosulfonyl, heterocyclosulfonyl, alkylsulfonyl, hydroxyarylcarbonyl, nitroaryl, optionally substituted aryl, optionally substituted heteroaryl, aralkylcarbonyl, heteroarylcarbonyl, arylcarbonyl, aminocarbonyl, and alkylcarbonyl;
wherein R⁴ together with the carbon atoms to which it is attached and the remainder of ring E forms a naphthyl radical;
or a pharmaceutically acceptable salt or an isomer or a prodrug thereof.

11. The method of claim 10, wherein:
n is an integer which is 0, 1, 2, 3 or 4;
G is O, S or NR^{b};
R¹ is H;
R^{b} is alkyl;
R² is selected from the group consisting of carboxyl, aminocarbonyl, alkylsulfonylaminocarbonyl and alkoxycarbonyl;
R³ is selected from the group consisting of haloalkyl, alkyl, aralkyl, cycloalkyl and aryl, wherein haloalkyl, alkyl, aralkyl, cycloalkyl, and aryl each is independently optionally substituted with one or more radicals selected from the group consisting of alkylthio, nitro and alkylsulfonyl; and
each R⁴ is independently selected from the group consisting of hydrido, halo, alkyl, aralkyl, alkoxy, aryloxy, heteroaryloxy, aralkyloxy, heteroaralkyloxy, haloalkyl, haloalkoxy, alkylamino, arylamino, aralkylamino, heteroarylamino, heteroarylalkylamino, nitro, amino, aminosulfonyl, alkylaminosulfonyl, arylaminosulfonyl, heteroarylaminosulfonyl, aralkylaminosulfonyl, heteroaralkylaminosulfonyl, heterocyclosulfonyl, alkylsulfonyl, optionally substituted aryl, optionally substituted heteroaryl, aralkylcarbonyl, heteroarylcarbonyl, arylcarbonyl, aminocarbonyl, and alkylcarbonyl; or wherein R⁴ together with ring E forms a naphthyl radical.

12. The method of claim 10, wherein:
n is an integer which is 0, 1, 2, 3 or 4;
G is oxygen or sulfur;
R¹ is H;
R² is carboxyl, lower alkyl, lower aralkyl or lower alkoxycarbonyl;
R³ is lower haloalkyl, lower cycloalkyl or phenyl; and
each R⁴ is H, halo, lower alkyl, lower alkoxy, lower haloalkyl, lower haloalkoxy, lower alkylamino, nitro, amino, aminosulfonyl, lower alkylaminosulfonyl, 5-membered heteroarylalkylaminosulfonyl, 6-membered heteroarylalkylaminosulfonyl, lower aralkylaminosulfonyl, 5-membered nitrogen-containing heterocyclosulfonyl, 6-membered-nitrogen containing heterocyclosulfonyl, lower alkylsulfonyl, optionally substituted phenyl, lower aralkylcarbonyl, or lower alkylcarbonyl; or
wherein R⁴ together with the carbon atoms to which it is attached and the remainder of ring E forms a naphthyl radical.

13. The method of claim 10, wherein:
R² is carboxyl;
R³ is lower haloalkyl; and
each R⁴ is H, halo, lower alkyl, lower haloalkyl, lower haloalkoxy, lower alkylamino, amino, aminosulfonyl, lower alkylaminosulfonyl, 5-membered heteroarylalkylaminosulfonyl, 6-membered heteroarylalkylaminosulfonyl, lower aralkylaminosulfonyl, lower alkylsulfonyl, 6-membered nitrogen-containing heterocyclosulfonyl, optionally substituted phenyl, lower aralkylcarbonyl, or lower alkylcarbonyl; or wherein R⁴ together with ring E forms a naphthyl radical.

14. The method of claim 10, wherein:
n is an integer which is 0, 1, 2, 3 or 4;
R³ is fluoromethyl, chloromethyl, dichloromethyl, trichloromethyl, pentafluoroethyl, heptafluoropropyl, difluoroethyl, difluoropropyl, dichloroethyl, dichloropropyl, difluoromethyl, or trifluoromethyl; and
each R⁴ is H, chloro, fluoro, bromo, iodo, methyl, ethyl, isopropyl, tert-butyl, butyl, isobutyl, pentyl, hexyl, methoxy, ethoxy, isopropyloxy, tertbutyloxy, trifluoromethyl, difluoromethyl, trifluoromethoxy, amino, N,N-dimethylamino, N,N-diethylamino, N-phenylmethylaminosulfonyl, N-phenylethylaminosulfonyl, N-(2-furylmethyl)aminosulfonyl, nitro, N,N-dimethylaminosulfonyl, aminosulfonyl, N-methylaminosulfonyl, N-ethylsulfonyl, 2,2-dimethylethylaminosulfonyl, N,N-dimethylaminosulfonyl, N-(2-methylpropyl)aminosulfonyl, N-morpholinosulfonyl, methylsulfonyl, benzylcarbonyl, 2,2-dimethylpropylcarbonyl, phenylacetyl or phenyl; or wherein R⁴ together with the carbon atoms to which it is attached and the remainder of ring E forms a naphthyl radical.

15. The method of claim 10 wherein the cyclooxygenase-2 selective inhibitor comprises a compound of the formula: 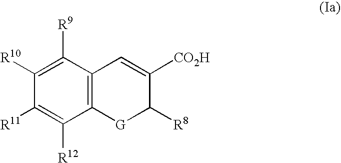
G is oxygen or sulfur;
R⁸ is trifluoromethyl or pentafluoroethyl;
R⁹ is H, chloro, or fluoro;
R¹⁰ is H, chloro, bromo, fluoro, iodo, methyl, tert-butyl, trifluoromethoxy, methoxy, benzylcarbonyl, dimethylaminosulfonyl, isopropylaminosulfonyl, methylaminosulfonyl, benzylaminosulfonyl, phenylethylaminosulfonyl, methylpropylaminosulfonyl, methylsulfonyl, or morpholinosulfonyl;
R¹¹ is H, methyl, ethyl, isopropyl, tert-butyl, chloro, methoxy, diethylamino, or phenyl; and
R¹² is H, chloro, bromo, fluoro, methyl, ethyl, tert-butyl, methoxy, or phenyl

16. The method of claim 10 wherein the cyclooxygenase-2 selective inhibitor, pharmaceutically acceptable salt, isomer or prodrug thereof is selected from the group consisting of:
6-chloro-2-trifluoromethyl-2H-1-benzopyran-3-carboxylic acid;
6-chloro-7-methyl-2-trifluoromethyl-2H-1-benzopyran-3-carboxylic acid;
8-(1-methylethyl)-2-trifluoromethyl-2H-1-benzopyran-3-carboxylic acid;
6-chloro-7-(1,1-dimethylethyl)-2-trifluoromethyl-2H-1-benzopyran-3-carboxylic acid;
6-chloro-8-(1-methylethyl)-2-trifluoromethyl-2H-1-benzopyran-3-carboxylic acid;
2-trifluoromethyl-3H-naphthopyran-3-carboxylic acid;
7-(1,1-dimethylethyl)-2-trifluoromethyl-2H-1-benzopyran-3-carboxylic acid;
6-bromo-2-trifluoromethyl-2H-1-benzopyran-3-carboxylic acid;
8-chloro-2-trifluoromethyl-2H-1-benzopyran-3-carboxylic acid;
6-trifluoromethoxy-2-trifluoromethyl-2H-1-benzopyran-3-carboxylic acid;
7-dichloro-2-trifluoromethyl-2H-1-benzopyran-3-carboxylic acid;
8-phenyl-2-trifluoromethyl-2H-1-benzopyran-3-carboxylic acid;
7,8-dimethyl-2-trifluoromethyl-2H-1-benzopyran-3-carboxylic acid;
6,8-bis(dimethylethyl)-2-trifluoromethyl-2H-1-benzopyran-3-carboxylic acid;
7-(1-methylethyl)-2-trifluoromethyl-2H-1-benzopyran-3-carboxylic acid;
7-phenyl-2-trifluoromethyl-2H-1-benzopyran-3-carboxylic acid;
6-chloro-7-ethyl-2-trifluoromethyl-2H-1-benzopyran-3-carboxylic acid;
6-chloro-8-ethyl-2-trifluoromethyl-2H-1-benzopyran-3-carboxylic acid;
6-chloro-7-phenyl-2-trifluoromethyl-2H-1-benzopyran-3-carboxylic acid;
6,7-dichloro-2-trifluoromethyl-2H-1-benzopyran-3-carboxylic acid;
6,8-dichloro-2-trifluoromethyl-2H-1-benzopyran-3-carboxylic acid;
2-trifluoromethyl-3H-naptho[2,1-b]pyran-3-carboxylic acid;
6-chloro-8-methyl-2-trifluoromethyl-2H-1-benzopyran-3-carboxylic acid;
8-chloro-6-methyl-2-trifluoromethyl-2H-1-benzopyran-3-carboxylic acid;
8-chloro-6-methoxy-2-trifluoromethyl-2H-1-benzopyran-3-carboxylic acid;
6-bromo-8-chloro-2-trifluoromethyl-2H-1-benzopyran-3-carboxylic acid;
8-bromo-6-fluoro-2-trifluoromethyl-2H-1-benzopyran-3-carboxylic acid;
8-bromo-6-methyl-2-trifluoromethyl-2H-1-benzopyran-3-carboxylic acid;
8-bromo-5-fluoro-2-trifluoromethyl-2H-1-benzopyran-3-carboxylic acid;
6-chloro-8-fluoro-2-trifluoromethyl-2H-1-benzopyran-3-carboxylic acid;
6-bromo-8-methoxy-2-tri fluoromethyl-2H-1-benzopyran-3-carboxylic acid;
6-[[(phenylmethyl)amino]sulfonyl]-2-trifluoromethyl-2H-1-benzopyran-3-carboxylic acid;
6-[(dimethylamino)sulfonyl]-2-trifluoromethyl-2H-1-benzopyran-3-carboxylic acid;
6-[(methylamino)sulfonyl]-2-trifluoromethyl-2H-1-benzopyran-3-carboxylic acid;
6-[(4-morpholino)sulfonyl]-2-trifluoromethyl-2H-1-benzopyran-3-carboxylic acid;
6-[(1,1-dimethylethyl)aminosulfonyl]-2-trifluoromethyl-2H-1-benzopyran-3-carboxylic acid;
6-[(2-methylpropyl)aminosulfonyl]-2-trifluoromethyl-2H-1-benzopyran-3-carboxylic acid;
6-methylsulfonyl-2-trifluoromethyl-2H-1-benzopyran-3-carboxylic acid;
8-chloro-6-[[(phenylmethyl)amino]sulfonyl]-2-trifluoromethyl-2H-1-benzopyran-3-carboxylic acid;
6-phenylacetyl-2-trifluoromethyl-2H-1-benzopyran-3-carboxylic acid;
6,8-dibromo-2-trifluoromethyl-2H-1-benzopyran-3-carboxylic acid;
8-chloro-5,6-dimethyl-2-trifluoromethyl-2H-1-benzopyran-3-carboxylic acid;
6,8-dichloro-(S)-2-trifluoromethyl-2H-1-benzopyran-3-carboxylic acid;
6-benzylsulfonyl-2-trifluoromethyl-2H-1-benzopyran-3-carboxylic acid;
6-[[N-(2-furylmethyl)amino]sulfonyl]-2-trifluoromethyl-2H-1-benzopyran-3-carboxylic acid;
6-[[N-(2-phenylethyl)amino]sulfonyl]-2-trifluoromethyl-2H-1-benzopyran-3-carboxylic acid;
6-iodo-2-trifluoromethyl-2H-1-benzopyran-3-carboxylic acid;
7-(1,1-dimethylethyl)-2-pentafluoroethyl-2H-1-benzopyran-3-carboxylic acid; and
6-chloro-2-trifluoromethyl-2H-1-benzothiopyran-3-carboxylic acid.

17. The method of claim 10 wherein the cyclooxygenase-2 selective inhibitor, pharmaceutically acceptable salt or prodrug thereof is selected from the group consisting of formulas: 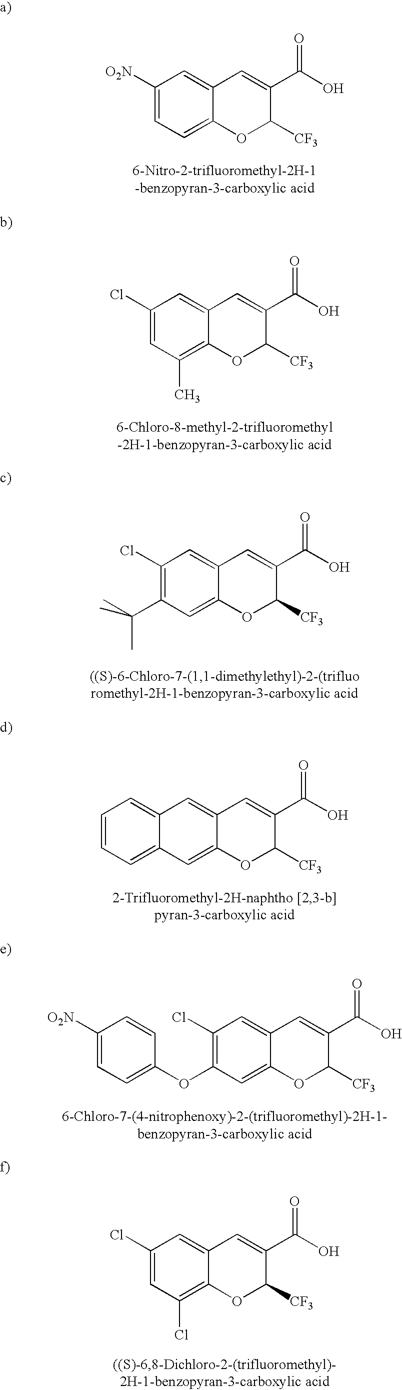 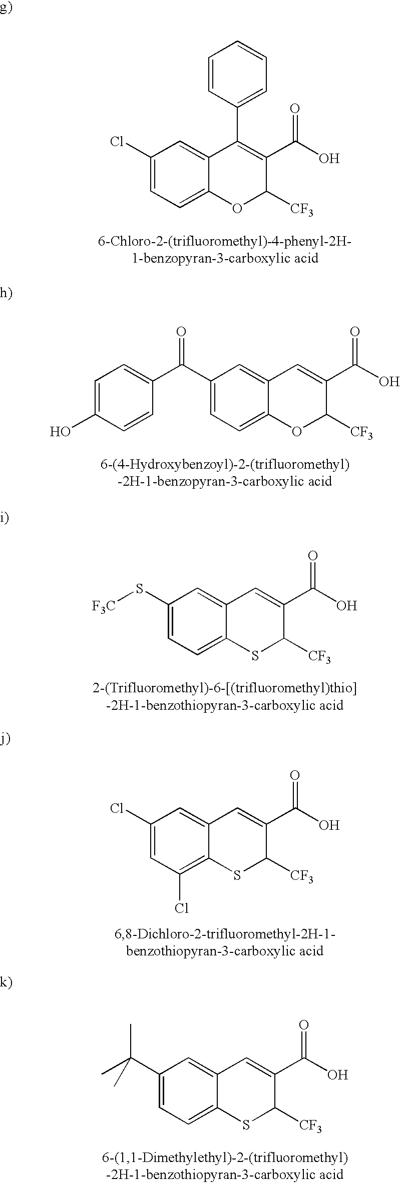 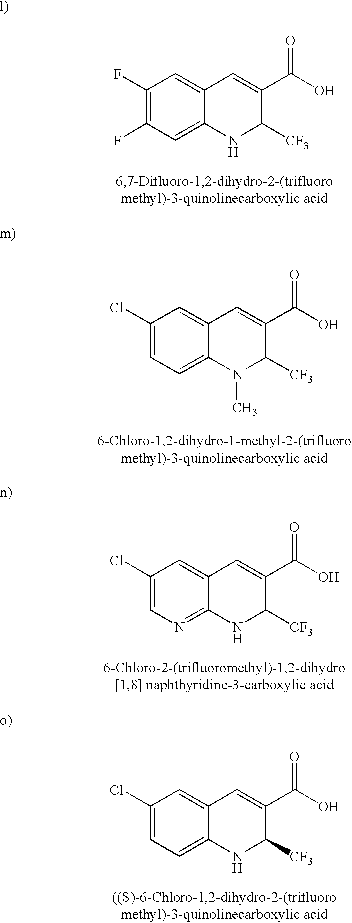
and any combination thereof.

18. The method of claim 1 wherein the cyclooxygenase inhibitor comprises a composition of the formula: 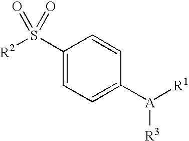
wherein A is selected from the group consisting of partially unsaturated or unsaturated heterocyclyl and partially unsaturated or unsaturated carbocyclic rings;
wherein R¹ is selected from the group consisting of heterocyclyl, cycloalkyl, cycloalkenyl and aryl, wherein R¹ is optionally substituted at a substitutable position with one or more radicals selected from alkyl, haloalkyl, cyano, carboxyl, alkoxycarbonyl, hydroxyl, hydroxyalkyl, haloalkoxy, amino, alkylamino, arylamino, nitro, alkoxyalkyl, alkylsulfinyl, halo, alkoxy and alkylthio;
wherein R² is selected from the group consisting of methyl or amino; and
wherein R³ is selected from the group consisting of a radical selected from H, halo, alkyl, alkenyl, alkynyl, oxo, cyano, carboxyl, cyanoalkyl, heterocyclyloxy, alkyloxy, alkylthio, alkylcarbonyl, cycloalkyl, aryl, haloalkyl, heterocyclyl, cycloalkenyl, aralkyl, heterocyclylalkyl, acyl, alkylthioalkyl, hydroxyalkyl, alkoxycarbonyl, arylcarbonyl, aralkylcarbonyl, aralkenyl, alkoxyalkyl, arylthioalkyl, aryloxyalkyl, aralkylthioalkyl, aralkoxyalkyl, alkoxyaralkoxyalkyl, alkoxycarbonylalkyl, aminocarbonyl, aminocarbonylalkyl, alkylaminocarbonyl, N-arylaminocarbonyl, N-alkyl-N-arylaminocarbonyl, alkylaminocarbonylalkyl, carboxyalkyl, alkylamino, N-arylamino, N-aralkylamino, N-alkyl-N-aralkylamino, N-alkyl-N-arylamino, aminoalkyl, alkylaminoalkyl, N-arylaminoalkyl, N-aralkylaminoalkyl, N-alkyl-N-aralkylaminoalkyl, N-alkyl-N-arylaminoalkyl, aryloxy, aralkoxy, arylthio, aralkylthio, alkylsulfinyl, alkylsulfonyl, aminosulfonyl, alkylaminosulfonyl, N-arylaminosulfonyl, arylsulfonyl, N-alkyl-N-arylaminosulfonyl; or a pharmaceutically acceptable salt or prodrug thereof.

19. The method of claim 1 wherein the cyclooxygenase-2 selective inhibitor pharmaceutically acceptable salt or prodrug thereof is selected from the group consisting of: 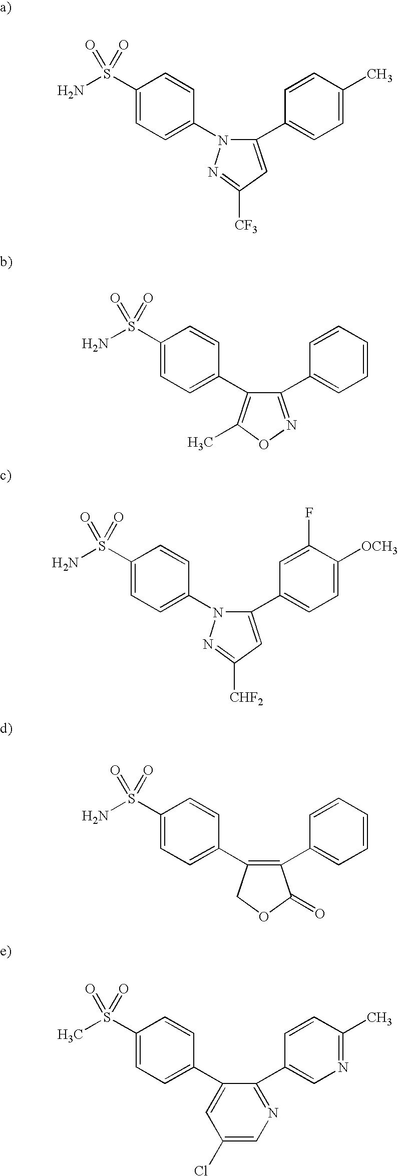 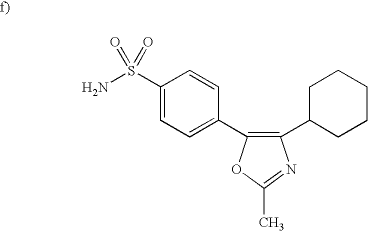
and any combination thereof.

20. The method of claim 1 wherein the cyclooxygenase-2 selective inhibitor or a pharmaceutically acceptable salt or prodrug thereof is selected from the group consisting of: 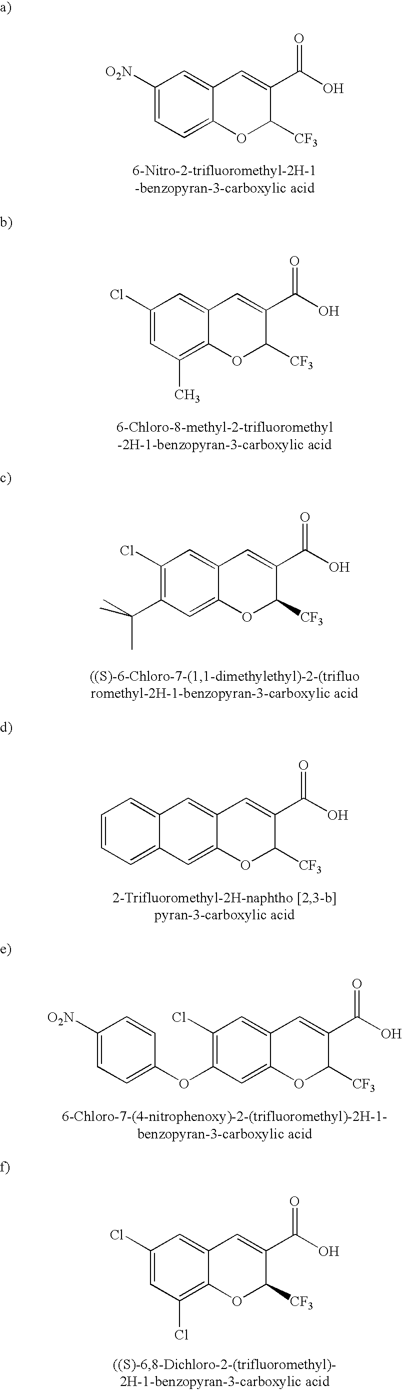 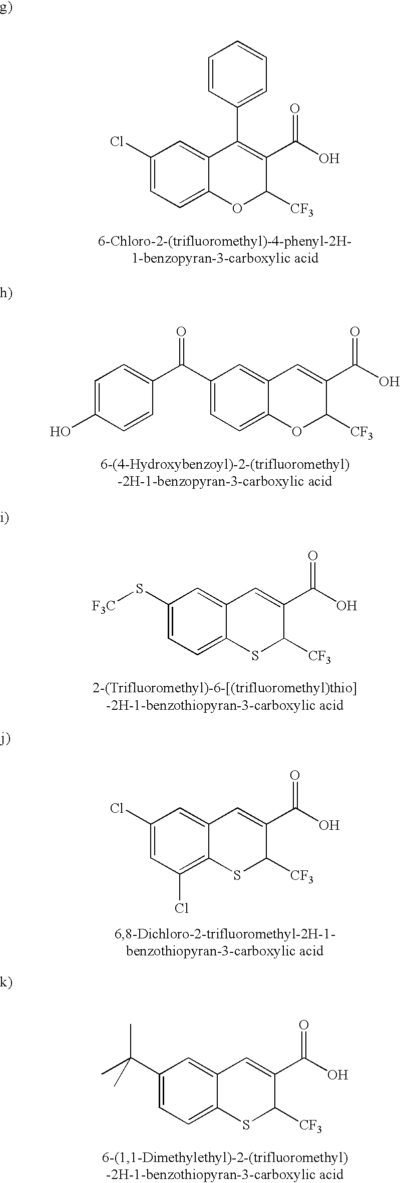 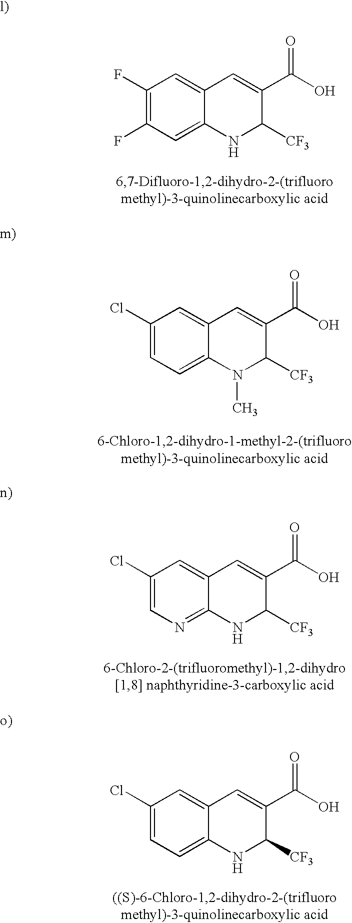 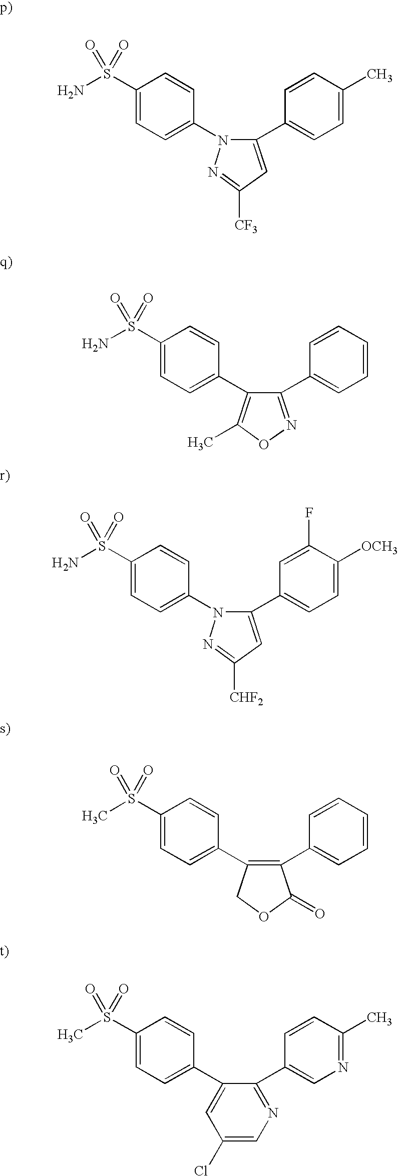 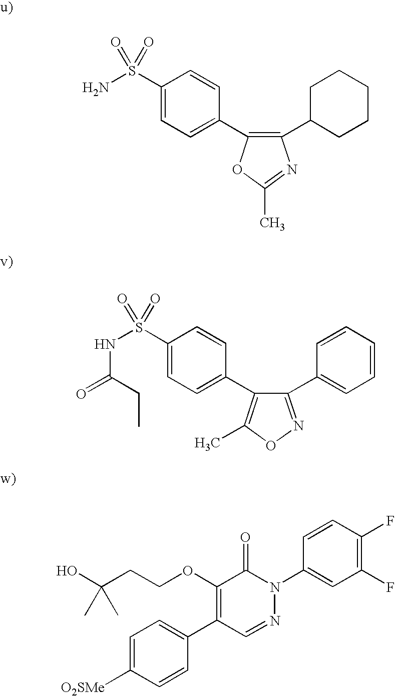
and any combination thereof.

21. The method of claim 1 wherein the cyclooxygenase-2 selective inhibitor comprises: 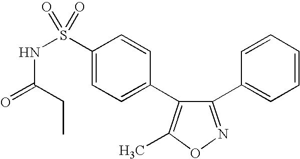
or a pharmaceutically acceptable salt or prodrug thereof.

22. The method of claim 1 wherein the cyclooxygenase-2 selective inhibitor comprises: 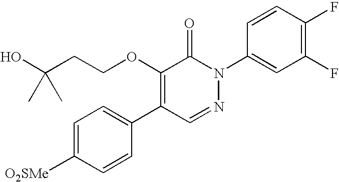
or a pharmaceutically acceptable salt or prodrug thereof.

23. The method of claim 1 wherein the cyclooxygenase-2 selective inhibitor comprises 4-[4-(methyl)-sulfonyl)phenyl]-3-phenyl-2(5H)-furanone, or a pharmaceutically acceptable salt or prodrug thereof.

24. The method of claim 1 wherein the cyclooxygenase-2 selective inhibitor comprises, 4-(5-methyl-3-phenyl-4-isoxazolyl), or a pharmaceutically acceptable salt or prodrug thereof.

25. The method of claim 1 wherein the cyclooxygenase-2 selective inhibitor comprises, 2-(6-methylpyrid-3-yl)-3-(4-methylsulfonylphenyl)-5-chloropyridine, or a pharmaceutically acceptable salt or prodrug thereof.

26. The method of claim 1 wherein the cyclooxygenase-2 selective inhibitor comprises, 4-[5-(4-methylphenyl)-3-(trifluoromethyl)-1H-pyrazol-1-yl], or a pharmaceutically acceptable salt or prodrug thereof.

27. The method of claim 1 wherein the cyclooxygenase-2 selective inhibitor comprises, N-[[4-(5-methyl-3-phenyl-4-isoxazolyl)phenyl]sulfonyl], or a pharmaceutically acceptable salt or prodrug thereof.

28. The method of claim 1 wherein the cyclooxygenase-2 selective inhibitor comprises, 4-[5-(3-fluoro-4-methoxyphenyl)-3-difluoromethyl)-1H-pyrazol-1-yl]benzenesulfonamide, or a pharmaceutically acceptable salt or prodrug thereof.

29. The method of claim 1 wherein the cyclooxygenase-2 selective inhibitor comprises, (S)-6,8-dichloro-2-(trifluoromethyl)-2H-1-benzopyran-3-carboxylic acid, or a pharmaceutically acceptable salt or prodrug thereof.

30. The method of claim 1 wherein the cyclooxygenase-2 selective inhibitor comprises, 2-(3,4-difluorophenyl)-4-(3-hydroxy-3-methylbutoxy)-5-[4-(methylsulfonyl)phenyl]-3(2H)-pyridzainone, or a pharmaceutically acceptable salt or prodrug thereof.

31. The method of claim 1 wherein the cyclooxygenase-2 selective inhibitor comprises a compound of the formula: 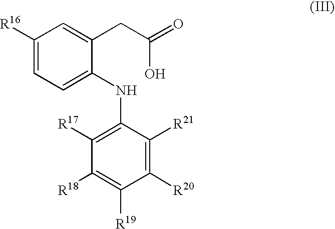
wherein:
R¹⁶ is methyl or ethyl;
R¹⁷ is chloro or fluoro;
R¹⁸ is hydrogen or fluoro;
R¹⁹ is hydrogen, fluoro, chloro, methyl, ethyl, methoxy, ethoxy or hydroxy;
R²⁰ is hydrogen or fluoro;
R²¹ is chloro, fluoro, trifluoromethyl or methyl, provided that R¹⁷, R¹⁸, R¹⁹ and R²⁰ are not all fluoro when R¹⁶ is ethyl and R¹⁹ is H; or an isomer, a pharmaceutically acceptable salt, ester, or prodrug thereof.

32. The method of claim 31 wherein:
R¹⁶ is ethyl;
R¹⁷ and R¹⁹ are chloro;
R¹⁸ and R²⁰ are hydrogen; and
and R²¹ is methyl.

33. The method of claim 1 wherein the cyclooxygenase-2 selective inhibitor comprises a compound of the formula: 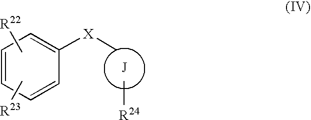
wherein:
X is O or S;
J is a carbocycle or a heterocycle;
R²² is NHSO₂CH₃ or F;
R²³ is H, NO₂, or F; and
R²⁴ is H, NHSO₂CH₃, or (SO₂CH₃)C₆H₄;
or an isomer, a pharmaceutically acceptable salt, an ester, or a prodrug thereof.

34. The method of claim 1 wherein the cyclooxygenase-2 selective inhibitor comprises a compound of the formula: 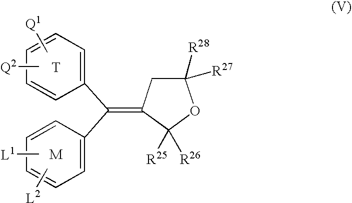
wherein:
T and M independently are phenyl, naphthyl, a radical derived from a heterocycle comprising 5 to 6 members and possessing from 1 to 4 heteroatoms, or a radical derived from a saturated hydrocarbon ring having from 3 to 7 carbon atoms;
Q¹, Q², L¹ or L² are independently hydrogen, halogen, lower alkyl having from 1 to 6 carbon atoms, trifluoromethyl, or lower methoxy having from 1 to 6 carbon atoms; and
at least one of Q¹, Q², L¹ or L² is in the para position and is —S(O)ₙ—R, wherein n is 0, 1, or 2 and R is a lower alkyl radical having 1 to 6 carbon atoms or a lower haloalkyl radical having from 1 to 6 carbon atoms, or an —SO₂NH₂; or,
Q¹ and Q² are methylenedioxy; or
L¹ and L² are methylenedioxy; and
R²⁵, R²⁶, R²⁷, and R²⁸ are independently hydrogen, halogen, lower alkyl radical having from 1 to 6 carbon atoms, lower haloalkyl radical having from 1 to 6 carbon atoms, or an aromatic radical selected from the group consisting of phenyl, naphthyl, thienyl, furyl and pyridyl; or,
R²⁵ and R²⁶ are 0; or
R²⁷ and R²⁸ are 0; or,
R²⁵, R²⁶, together with the carbon atom to which they are attached, form a saturated hydrocarbon ring having from 3 to 7 carbon atoms; or,
R²⁷, R²⁸, together with the carbon atom to which they are attached, form a saturated hydrocarbon ring having from 3 to 7 carbon atoms;
or an isomer, a pharmaceutically acceptable salt, an ester, or a prodrug thereof.

35. The method of claim 1 wherein the cyclooxygenase-2 selective inhibitor, pharmaceutically acceptable salt, isomer, or prodrug thereof is selected from the group consisting of:
3-[(3-Chloro-phenyl)-(4-methanesulfony-phenyl)-methylene]-dihydro-furan-2-one;
8-acetyl-3-(4-fluorophenyl)-2-(4-methylsulfonyl)phenyl-imidazo[1,2-a);
5,5-dimethyl-4-(4-methylsulfonyl)phenyl-3-phenyl-2-(5H)-furanone;
5-(4-fluorophenyl)-1-[4-(methylsulfonyl)phenyl]-3-(trifluoromethyl)pyrazole;
4-(4-fluorophenyl)-5-[4-(methylsulfonyl)phenyl]-1-phenyl-3-(trifluoromethyl)pyrazole;
4-(5-(4-chlorophenyl)-3-(4-methoxyphenyl)-1H-pyrazol-1-yl)benzenesulfonamide;
4-(3,5-bis(4-methylphenyl)-1H-pyrazol-1-yl)benzenesulfonamide;
4-(5-(4-chlorophenyl)-3-phenyl-1H-pyrazol-1-yl)benzenesulfonamide;
4-(3,5-bis(4-methoxyphenyl)-1H-pyrazol-1-yl)benzenesulfonamide;
4-(5-(4-chlorophenyl)-3-(4-methylphenyl)-1H-pyrazol-1-yl)benzenesulfonamide;
4-(5-(4-chlorophenyl)-3-(4-nitrophenyl)-1H-pyrazol-1-yl)benzenesulfonamide;
4-(5-(4-chlorophenyl)-3-(5-chloro-2-thienyl)-1H-pyrazol-1-yl)benzenesulfonamide;
4-(4-chloro-3,5-diphenyl-1H-pyrazol-1-yl)benzenesulfonamide;
4-[5-(4-chlorophenyl]-3-(trifluoromethyl)-1H-pyrazol-1-yl]benzenesulfonamide;
4-[5-phenyl-3-(trifluoromethyl)-1H-pyrazol-1-yl]benzenesulfonamide;
4-[5-(4-fluorophenyl)-3-(trifluoromethyl)-1H-pyrazol-1-yl]benzenesulfonamide;
4-[5-(4-methoxyphenyl)-3-(trifluoromethyl)-1H-pyrazol-1-yl]benzenesulfonamide;
4-[5-(4-chlorophenyl)-3-(difluoromethyl)-1H-pyrazol-1-yl]benzenesulfonamide;
4-[5-(4-methylphenyl)-3-(trifluoromethyl)-1H-pyrazol-1-yl]benzenesulfonamide;
4-[4-chloro-5-(4-chlorophenyl)-3-(trifluoromethyl)-1H-pyrazol-1-yl]benzenesulfonamide;
4-[3-(difluoromethyl)-5-(4-methylphenyl)-1H-pyrazol-1-yl]benzenesulfonamide;
4-[3-(difluoromethyl)-5-phenyl-1H-pyrazol-1-yl]benzenesulfonamide;
4-[3-(difluoromethyl)-5-(4-methoxyphenyl)-1H-pyrazol-1-yl]benzenesulfonamide;
4-[3-cyano-5-(4-fluorophenyl)-1H-pyrazol-1-yl]benzenesulfonamide;
4-[3-(difluoromethyl)-5-(3-fluoro-4-methoxyphenyl)-1H-pyrazol-1-yl]benzenesulfonamide;
4-[5-(3-fluoro-4-methoxyphenyl)-3-(trifluoromethyl)-1H-pyrazol-1-yl]benzenesulfonamide;
4-[4-chloro-5-phenyl-1H-pyrazol-1-yl]benzenesulfonamide;
4-[5-(4-chlorophenyl)-3-(hydroxymethyl)-1H-pyrazol-1-yl]benzenesulfonamide;
4-[5-(4-(N,N-dimethylamino)phenyl)-3-(trifluoromethyl)-1H-pyrazol-1-yl]benzenesulfonamide;
5-(4-fluorophenyl)-6-[4-(methylsulfonyl)phenyl]spiro[2.4]hept-5-ene;
4-[6-(4-fluorophenyl)spiro[2.4]hept-5-en-5-yl]benzenesulfonamide;
6-(4-fluorophenyl)-7-[4-(methylsulfonyl)phenyl]spiro[3.4]oct-6-ene;
5-(3-chloro-4-methoxyphenyl)-6-[4-(methylsulfonyl)phenyl]spiro[2.4]hept-5-ene;
4-[6-(3-chloro-4-methoxyphenyl)spiro[2.4]hept-5-en-5-yl]benzenesulfonamide;
5-(3,5-dichloro-4-methoxyphenyl)-6-[4-(methylsulfonyl)phenyl]spiro[2.4]hept-5-ene;
5-(3-chloro-4-fluorophenyl)-6-[4-(methylsulfonyl)phenyl]spiro[2.4]hept-5-ene;
4-[6-(3,4-dichlorophenyl)spiro[2.4]hept-5-en-5-yl]benzenesulfonamide;
2-(3-chloro-4-fluorophenyl)-4-(4-fluorophenyl)-5-(4-methylsulfonylphenyl)thiazole;
2-(2-chlorophenyl)-4-(4-fluorophenyl)-5-(4-methylsulfonylphenyl)thiazole;
5-(4-fluorophenyl)-4-(4-methylsulfonylphenyl)-2-methylthiazole;
4-(4-fluorophenyl)-5-(4-methylsulfonylphenyl)-2-trifluoromethylthiazole;
4-(4-fluorophenyl)-5-(4-methylsulfonylphenyl)-2-(2-thienyl)thiazole;
4-(4-fluorophenyl)-5-(4-methylsulfonylphenyl)-2-benzylaminothiazole;
4-(4-fluorophenyl)-5-(4-methylsulfonylphenyl)-2-(1-propylamino)thiazole;
2-[(3,5-dichlorophenoxy)methyl)-4-(4-fluorophenyl)-5-[4-(methylsulfonyl)phenyl]thiazole;
5-(4-fluorophenyl)-4-(4-methylsulfonylphenyl)-2-trifluoromethylthiazole;
1-methylsulfonyl-4-[1,1-dimethyl-4-(4-fluorophenyl)cyclopenta-2,4-dien-3-yl]benzene;
4-[4-(4-fluorophenyl)-1,1-dimethylcyclopenta-2,4-dien-3-yl]benzenesulfonamide;
5-(4-fluorophenyl)-6-[4-(methylsulfonyl)phenyl]spiro[2.4]hepta-4,6-diene;
4-[6-(4-fluorophenyl)spiro[2.4]hepta-4,6-dien-5-yl]benzenesulfonamide;
6-(4-fluorophenyl)-2-methoxy-5-[4-(methylsulfonyl)phenyl]-pyridine-3-carbonitrile;
2-bromo-6-(4-fluorophenyl)-5-[4-(methylsulfonyl)phenyl]-pyridine-3-carbonitrile;
6-(4-fluorophenyl)-5-[4-(methylsulfonyl)phenyl]-2-phenyl-pyridine-3-carbonitrile;
4-[2-(4-methylpyridin-2-yl)-4-(trifluoromethyl)-1H-imidazol-1-yl]benzenesulfonamide;
4-[2-(5-methylpyridin-3-yl)-4-(trifluoromethyl)-1H-imidazol-1-yl]benzenesulfonamide;
4-[2-(2-methylpyridin-3-yl)-4-(trifluoromethyl)-1H-imidazol-1-yl]benzenesulfonamide;
3-[1-[4-(methylsulfonyl)phenyl]-4-(trifluoromethyl)-1H-imidazol-2-yl]pyridine;
2-[1-[4-(methylsulfonyl)phenyl-4-(trifluoromethyl)-1H-imidazol-2-yl]pyridine;
2-methyl-4-[1-[4-(methylsulfonyl)phenyl-4-(trifluoromethyl)-1H-imidazol-2-yl]pyridine;
2-methyl-6-[1-[4-(methylsulfonyl)phenyl-4-(trifluoromethyl)-1H-imidazol-2-yl]pyridine;
4-[2-(6-methylpyridin-3-yl)-4-(trifluoromethyl)-1H-imidazol-1-yl]benzenesulfonamide;
2-(3,4-difluorophenyl)-1-[4-(methylsulfonyl)phenyl]-4-(trifluoromethyl)-1H-imidazole;
4-[2-(4-methylphenyl)-4-(trifluoromethyl)-1H-imidazol-1-yl]benzenesulfonamide;
2-(4-chlorophenyl)-1-[4-(methylsulfonyl)phenyl]-4-methyl-1H-imidazole;
2-(4-chlorophenyl)-1-[4-(methylsulfonyl)phenyl]-4-phenyl-1H-imidazole;
2-(4-chlorophenyl)-4-(4-fluorophenyl)-1-[4-(methylsulfonyl)phenyl]-1H-imidazole;
2-(3-fluoro-4-methoxyphenyl)-1-[4-(methylsulfonyl)phenyl-4-(trifluoromethyl)-1H-imidazole;
1-[4-(methylsulfonyl)phenyl]-2-phenyl-4-trifluoromethyl-1H-imidazole;
2-(4-methylphenyl)-1-[4-(methylsulfonyl)phenyl]-4-trifluoromethyl-1H-imidazole;
4-[2-(3-chloro-4-methylphenyl)-4-(trifluoromethyl)-1H-imidazol-1-yl]benzenesulfonamide;
2-(3-fluoro-5-methylphenyl)-1-[4-(methylsulfonyl)phenyl]-4-(trifluoromethyl)-1H-imidazole;
4-[2-(3-fluoro-5-methylphenyl)-4-(trifluoromethyl)-1H-imidazol-1-yl]benzenesulfonamide;
2-(3-methylphenyl)-1-[4-(methylsulfonyl)phenyl]-4-trifluoromethyl-1H-imidazole;
4-[2-(3-methylphenyl)-4-trifluoromethyl-1H-imidazol-1-yl]benzenesulfonamide;
1-[4-(methylsulfonyl)phenyl]-2-(3-chlorophenyl)-4-trifluoromethyl-1H-imidazole;
4-[2-(3-chlorophenyl)-4-trifluoromethyl-1H-imidazol-1-yl]benzenesulfonamide;
4-[2-phenyl-4-trifluoromethyl-1H-imidazol-1-yl]benzenesulfonamide;
4-[2-(4-methoxy-3-chlorophenyl)-4-trifluoromethyl-1H-imidazol-1-yl]benzenesulfonamide;
1-allyl-4-(4-fluorophenyl)-3-[4-(methylsulfonyl)phenyl]-5-(trifluoromethyl)-1H-pyrazole;
4-[1-ethyl-4-(4-fluorophenyl)-5-(trifluoromethyl)-1H-pyrazol-3-yl]benzenesulfonamide;
N-phenyl-[4-(4-fluorophenyl)-3-[4-(methylsulfonyl)phenyl]-5-(trifluoromethyl)-1H-pyrazol-1-yl]acetamide;
ethyl [4-(4-fluorophenyl)-3-[4-(methylsulfonyl)phenyl]-5-(trifluoromethyl)-1H-pyrazol-1-yl]acetate;
4-(4-fluorophenyl)-3-[4-(methylsulfonyl)phenyl]-1-(2-phenylethyl)-1H-pyrazole;
4-(4-fluorophenyl)-3-[4-(methylsulfonyl)phenyl]-1-(2-phenylethyl)-5-(trifluoromethyl)pyrazole;
1-ethyl-4-(4-fluorophenyl)-3-[4-(methylsulfonyl)phenyl]-5-(trifluoromethyl)-1H-pyrazole;
5-(4-fluorophenyl)-4-(4-methylsulfonylphenyl)-2-trifluoromethyl-1H-imidazole;
4-[4-(methylsulfonyl)phenyl]-5-(2-thiophenyl)-2-(trifluoromethyl)-1H-imidazole;
5-(4-fluorophenyl)-2-methoxy-4-[4-(methylsulfonyl)phenyl]-6-(trifluoromethyl)pyridine;
2-ethoxy-5-(4-fluorophenyl)-4-[4-(methylsulfonyl)phenyl]-6-(trifluoromethyl)pyridine;
5-(4-fluorophenyl)-4-[4-(methylsulfonyl)phenyl]-2-(2-propynyloxy)-6-(trifluoromethyl)pyridine;
2-bromo-5-(4-fluorophenyl)-4-[4-(methylsulfonyl)phenyl]-6-(trifluoromethyl)pyridine;
4-[2-(3-chloro-4-methoxyphenyl)-4,5-difluorophenyl]benzenesulfonamide;
1-(4-fluorophenyl)-2-[4-(methylsulfonyl)phenyl]benzene;
5-difluoromethyl-4-(4-methylsulfonylphenyl)-3-phenylisoxazole;
4-[3-ethyl-5-phenylisoxazol-4-yl]benzenesulfonamide;
4-[5-difluoromethyl-3-phenylisoxazol-4-yl]benzenesulfonamide;
4-[5-hydroxymethyl-3-phenylisoxazol-4-yl]benzenesulfonamide;
4-[5-methyl-3-phenyl-isoxazol-4-yl]benzenesulfonamide;
1-[2-(4-fluorophenyl)cyclopenten-1-yl]-4-(methylsulfonyl)benzene;
1-[2-(4-fluoro-2-methylphenyl)cyclopenten-1-yl]-4-(methylsulfonyl)benzene;
1-[2-(4-chlorophenyl)cyclopenten-1-yl]-4-(methylsulfonyl)benzene;
1-[2-(2,4-dichlorophenyl)cyclopenten-1-yl]-4-(methylsulfonyl)benzene;
1-[2-(4-trifluoromethylphenyl)cyclopenten-1-yl]-4-(methylsulfonyl)benzene;
1-[2-(4-methylthiophenyl)cyclopenten-1-yl]-4-(methylsulfonyl)benzene;
1-[2-(4-fluorophenyl)-4,4-dimethylcyclopenten-1-yl]-4-(methylsulfonyl)benzene;
4-[2-(4-fluorophenyl)-4,4-dimethylcyclopenten-1-yl]benzenesulfonamide;
1-[2-(4-chlorophenyl)-4,4-dimethylcyclopenten-1-yl]-4-(methylsulfonyl)benzene;
4-[2-(4-chlorophenyl)-4,4-dimethylcyclopenten-1-yl]benzenesulfonamide;
4-[2-(4-fluorophenyl)cyclopenten-1-yl]benzenesulfonamide;
4-[2-(4-chlorophenyl)cyclopenten-1-yl]benzenesulfonamide;
1-[2-(4-methoxyphenyl)cyclopenten-1-yl]-4-(methylsulfonyl)benzene;
1-[2-(2,3-difluorophenyl)cyclopenten-1-yl]-4-(methylsulfonyl)benzene;
4-[2-(3-fluoro-4-methoxyphenyl)cyclopenten-1-yl]benzenesulfonamide;
1-[2-(3-chloro-4-methoxyphenyl)cyclopenten-1-yl]-4-(methylsulfonyl)benzene;
4-[2-(3-chloro-4-fluorophenyl)cyclopenten-1-yl]benzenesulfonamide;
4-[2-(2-methylpyridin-5-yl)cyclopenten-1-yl]benzenesulfonamide;
ethyl 2-[4-(4-fluorophenyl)-5-[4-(methylsulfonyl) phenyl]oxazol-2-yl]-2-benzyl-acetate;
2-[4-(4-fluorophenyl)-5-[4-(methylsulfonyl)phenyl]oxazol-2-yl]acetic acid;
2-(tert-butyl)-4-(4-fluorophenyl)-5-[4-(methylsulfonyl)phenyl]oxazole;
4-(4-fluorophenyl)-5-[4-(methylsulfonyl)phenyl]-2-phenyloxazole;
4-(4-fluorophenyl)-2-methyl-5-[4-(methylsulfonyl)phenyl]oxazole;
4-[5-(3-fluoro-4-methoxyphenyl)-2-trifluoromethyl-4-oxazolyl]benzenesulfonamide;
6-chloro-7-(1,1-dimethylethyl)-2-trifluoromethyl-2H-1-benzopyran-3-carboxylic acid;
6-chloro-8-methyl-2-trifluoromethyl-2H-1-benzopyran-3-carboxylic acid;
5,5-dimethyl-3-(3-fluorophenyl)-4-methylsulfonyl-2(5H)-furanone;
6-chloro-2-trifluoromethyl-2H-1-benzothiopyran-3-carboxylic acid;
4-[5-(4-chlorophenyl)-3-(trifluoromethyl)-1H-pyrazol-1-yl]benzenesulfonamide;
4-[5-(4-methylphenyl)-3-(trifluoromethyl)-1H-pyrazol-1-yl]benzenesulfonamide;
4-[5-(3-fluoro-4-methoxyphenyl)-3-(difluoromethyl)-1H-pyrazol-1-yl]benzenesulfonamide;
3-[1-[4-(methylsulfonyl)phenyl]-4-trifluoromethyl-1H-imidazol-2-yl]pyridine;
2-methyl-5-[1-[4-(methylsulfonyl)phenyl]-4-trifluoromethyl-1H-imidazol-2-yl]pyridine;
4-[2-(5-methylpyridin-3-yl)-4-(trifluoromethyl)-1H-imidazol-1-yl]benzenesulfonamide;
4-[5-methyl-3-phenylisoxazol-4-yl]benzenesulfonamide;
4-[5-hydroxymethyl-3-phenylisoxazol-4-yl]benzenesulfonamide;
[2-trifluoromethyl-5-(3,4-difluorophenyl)-4-oxazolyl]benzenesulfonamide;
4-[2-methyl-4-phenyl-5-oxazolyl]benzenesulfonamide;
4-[5-(2-fluoro-4-methoxyphenyl)-2-trifluoromethyl-4-oxazolyl]benzenesulfonamide;
[2-(2-chloro-6-fluoro-phenylamino)-5-methyl-phenyl]-acetic acid;
N-(4-Nitro-2-phenoxy-phenyl)-methanesulfonamide or nimesulide;
N-[6-(2,4-difluoro-phenoxy)-1-oxo-indan-5-yl]-methanesulfonamide;
N-[6-(2,4-Difluoro-phenylsulfanyl)-1-oxo-1H-inden-5-yl]-methanesulfonamide, soldium salt;
N-[5-(4-fluoro-phenylsulfanyl)-thiophen-2-yl]-methanesulfonamide;
3-(3,4-Difluoro-phenoxy)-4-(4-methanesulfonyl-phenyl)-5-methyl-5-(2,2,2-trifluoroethyl)-5H-furan-2-one;
(5Z)-2-amino-5-[[3,5-bis(1,1-dimethylethyl)-4-hydroxyphenyl]methylene]-4(5H)-thiazolone;
N-[3-(formylamino)-4-oxo-6-phenoxy-4H-1-benzopyran-7-yl]-methanesulfonamide;
(6aR, 10aR)-3-(1,1-dimethylheptyl)-6a,7,10,10a-tetrahydro-1-hydroxy-6,6-dimethyl-6H-dibenzo[b,d]pyran-9-carboxylic acid;
4-[[3,5-bis(1,1-dimethylethyl)-4-hydroxyphenyl]methylene]dihydro-2-methyl-2H-1,2-oxazin-3(4H)-one;
6-dioxo-9H-purin-8-yl-cinnamic acid;
4-[4-(methyl)-sulfonyl)phenyl]-3-phenyl-2(5H)-furanone;
4-(5-methyl-3-phenyl-4-isoxazolyl);
2-(6-methylpyrid-3-yl)-3-(4-methylsulfonylphenyl)-5-chloropyridine;
4-[5-(4-methylphenyl)-3-(trifluoromethyl)-1H-pyrazol-1-yl];
N-[[4-(5-methyl-3-phenyl-4-isoxazolyl)phenyl]sulfonyl];
4-[5-(3-fluoro-4-methoxyphenyl)-3-difluoromethyl)-1H-pyrazol-1-yl]benzenesulfonamide;
(S)-6,8-dichloro-2-(trifluoromethyl)-2H-1-benzopyran-3-carboxylic acid;
2-(3,4-difluorophenyl)-4-(3-hydroxy-3-methylbutoxy)-5-[4-(methylsulfonyl)phenyl]-3 (2H)-pyridzainone;
2-trifluoromethyl-3H-naptho[2,1-b]pyran-3-carboxylic acid;
6-chloro-7-(1,1-dimethylethyl)-2-trifluoromethyl-2H-1-benzopyran-3-carboxylic acid; and
[2-(2,4-dichloro-6-ethyl-3,5-dimethyl-phenylamino)-5-propyl-phenyl]-acetic acid.

36. The method of claim 1 wherein the amyloid beta vaccine is a peptide vaccine.

37. The method of claim 1 wherein the amyloid beta vaccine is a nucleic acid vaccine.

38. The method of claim 36 wherein the amyloid beta vaccine comprises at least one amyloid beta peptide selected from Abeta (1-43), or a fragment, variant, or analog thereof.

39. The method of claim 38 wherein the amyloid beta peptide is selected from the group consisting of Abeta (1-42), Abeta (1-43), Abeta (1-40), Abeta (1-39), Abeta (1-41), Abeta (1-28), Abeta (1-16), Abeta (25-35), Abeta (29-39), Abeta (29-40), Abeta (29-41), Abeta (29-42), Abeta (29-43), Abeta (26-42), Abeta (26-43), and Abeta (35-43).

40. The method of claim 39 wherein the amyloid beta peptide is Abeta (1-42).

41. The method of claim 38 wherein the amyloid beta vaccine further comprises an adjuvant.

42. The method of claim 41 wherein the adjuvant is aluminum hydroxide.

43. The method of claim 41 wherein the adjuvant is aluminum phosphate.

44. The method of claim 36 wherein the amyloid beta vaccine is a monovalent vaccine.

45. The method of claim 36 wherein the amyloid beta vaccine is a multivalent vaccine.

46. The method of claim 1 wherein the amyloid beta vaccine is administered prior to the administration of the Cox-2 inhibitor.

47. The method of claim 1 wherein the Cox-2 inhibitor is administered during time intervals between each amyloid beta vaccination.

48. The method of claim 1 wherein the amyloid beta vaccine is administered following the administration of the Cox-2 inhibitor.

49. The method of claim 1 wherein the amyloid beta vaccine is administered for the life of the subject.

50. The method of claim 1 wherein the subject is a mammal.

51. The method of claim 50 wherein the mammal is a human being.

52. A composition comprising an amyloid beta vaccine and a cyclooxygenase-2 selective inhibitor or a pharmaceutically acceptable salt or prodrug thereof.

53. The composition of claim 52 wherein the cyclooxygenase-2 selective inhibitor comprises a chromene compound.

54. The composition of claim 53 wherein the chromene compound is a benzopyran or substituted benzopyran analog.

55. The composition of claim 54 wherein the benzopyran or substituted benzopyran analog is selected from the group consisting of benzothiopyrans, dihydroquinolines and dihydronaphthalenes.

56. The composition of claim 52 wherein the cyclooxygenase-2 selective inhibitor comprises a tricyclic compound.

57. The composition of claim 56 wherein the tricyclic compound comprises a benzenesulfonamide or methylsulfonylbenzene.

58. The composition of claim 52 wherein the cyclooxygenase-2 selective inhibitor comprises a phenyl acetic acid derivative.

59. The composition of claim 52 wherein the amyloid beta vaccine is a peptide vaccine.

60. The composition of claim 52 wherein the amyloid beta vaccine is a nucleic acid vaccine.

61. The composition of claim 59 wherein the amyloid beta vaccine comprises at least one amyloid beta peptide selected from Abeta (1-43), or a fragment, variant, or analog thereof.

62. The composition of claim 61 wherein the amyloid beta peptide is selected from the group consisting of Abeta (1-42), Abeta (1-43), Abeta (1-40), Abeta (1-39), Abeta (1-41), Abeta (1-28), Abeta (1-16), Abeta (25-35), Abeta (29-39), Abeta (29-40), Abeta (29-41), Abeta (29-42), Abeta (29-43), Abeta (26-42), Abeta (26-43), and Abeta (35-43).

63. The composition of claim 62 wherein the amyloid beta peptide is Abeta (1-42).

64. The composition of claim 61 wherein the amyloid beta vaccine further comprises an adjuvant.

65. The composition of claim 64 wherein the adjuvant is aluminum hydroxide.

66. The composition of claim 64 wherein the adjuvant is aluminum phosphate.

67. The composition of claim 59 wherein the amyloid beta vaccine is a monovalent vaccine.

68. The composition of claim 59 wherein the amyloid beta vaccine is a multivalent vaccine.

69. The method of claim 1 wherein the vaccine is administered by a route selected from the group consisting of oral, intramuscular, intravenous, subcutaneous, intradermal, and intraperitoneal.

70. The method of claim 1 wherein the cyclooxygenase-2 selective inhibitor comprises a compound of the formula: [chemical structure]
